(19) United States
(12) Patent Application Publication
(10) Pub. No.: US 2023/0255200 A1
(43) Pub. Date: Aug. 17, 2023

(54) USE OF STROBILURIN TYPE COMPOUNDS FOR COMBATING PHYTOPATHOGENIC FUNGI CONTAINING AN AMINO ACID SUBSTITUTION F129L IN THE MITOCHONDRIAL CYTOCHROME B PROTEIN CONFERRING RESISTANCE TO QO INHIBITORS VI

(71) Applicant: BASF SE, Ludwigshafen (DE)

(72) Inventors: Sarang Kulkarni, Pune (IN); Chandan Dey, Navi Mumbai (IN); Manojkumar Poonoth, Ludwigshafen (DE); Rakesh Rath, Navi Mumbai (IN); Ronan Le Vezouet, Ludwigshafen (DE); Christian Harald Winter, Ludwigshafen (DE); Andreas Koch, Limburgerhof (DE); Marcus Fehr, Limburgerhof (DE); Vanessa Tegge, Limburgerhof (DE); Wassilios Grammenos, Ludwigshafen (DE); Smriti Khanna, Navi Mumbai (IN)

(21) Appl. No.: 18/014,163

(22) PCT Filed: Jun. 29, 2021
(86) PCT No.: PCT/EP2021/067891

§ 371 Date: Jan. 2, 2023

(30) Foreign Application Priority Data
Jul. 8, 2020 (IN) 202021028966
Aug. 28, 2020 (EP) 20193416.3
Jun. 8, 2021 (EP) 21178121.6

(51) Int. Cl. A01N 37/28; C07C 251/60; C07C 251/58; A01N 37/06; A01P 3/00

(52) U.S. Cl. CPC A01N 37/28; C07C 251/60; C07C 251/58; A01N 37/06; A01P 3/00

(57) ABSTRACT

The present invention relates to the use of strobilurin type compounds of formula (I) and the N-oxides and the salts thereof for combating phytopathogenic fungi containing an amino acid substitution F129L in the mitochondrial cytochrome b protein (also referred to as F129L mutation in the mitochondrial cytochrome b gene) conferring resistance to Qo inhibitors, and to methods for combating such fungi. The invention also relates to novel compounds, processes for preparing these compounds, to compositions comprising at least one such compound, and to seeds coated with at least one such compound.

The present invention relates the use of strobilurin type compounds of formula I and the N-oxides and the salts thereof for combating phytopathogenic fungi containing an amino acid substitution F129L in the mitochondrial cytochrome b protein (also referred to as F129L mutation in the mitochondrial cytochrome b gene) conferring resistance to Qo inhibitors (QoI), and to methods for combating such fungi. The invention also relates to novel compounds, processes for preparing these compounds, to compositions comprising at least one such compound, to plant health applications, and to seeds coated with at least one such compound. The present invention also relates to a method for controlling soybean rust fungi (Phakopsora pachyrhizi) with the amino acid substitution F129L in the mitochondrial cytochrome b protein.

“Qo inhibitor,” as used herein, includes any substance that is capable of diminishing and/or inhibiting respiration by binding to a ubihydroquinone oxidation center of a cytochrome bc₁ complex in mitochondria. The oxidation center is typically located on the outer side of the inner mitochondrial membrane. Many of these compounds are also known as strobilurin-type or strobilurin analogue compounds.

The mutation F129L in the mitochondrial cytochrome b (CYTB) gene shall mean any substitution of nucleotides of codon 129 encoding “F” (phenylalanine; e.g. TTT or TTC) that leads to a codon encoding “L” (leucine; e.g. TTA, TTG, TTG, CTT, CTC, CTA or CTG), for example the substitution of the first nucleotide of codon 129 ‘T’ to ‘C’ (TTT to CTT), in the CYTB (cytochrome b) gene resulting in a single amino acid substitution in the position 129 from F to L in the cytochrome b protein. Such F129L mutation is known to confer resistance to Qo inhibitors QoI fungicides, often referred to as strobilurin-type fungicides, are conventionally used to control a number of fungal pathogens in crops. Qo inhibitors typically work by inhibiting respiration by binding to a ubihydroquinone oxidation center of a cytochrome bc₁ complex (electron transport complex III) in mitochondria. Said oxidation center is located on the outer side of the inner mitochondrial membrane. A prime example of the use of QoIs includes the use of, for example, strobilurins on wheat for the control of Septoria tritici (also known as Mycosphaerella graminicola), which is the cause of wheat leaf blotch. Unfortunately, widespread use of such QoIs has resulted in the selection of mutant pathogens which are resistant to such QoIs (Gisi et al., Pest Manag Sci 56, 833-841, (2000)). Resistance to QoIs has been detected in several phytopathogenic fungi such as Blumeria graminis, Mycosphaerella fijiensis, Pseudoperonspora cubensis or Venturia inaequalis. The major part of resistance to QoIs in agricultural uses has been attributed to pathogens containing a single amino acid residue substitution G143A in the cytochrome b gene for their cytochrome bc₁ complex, the target protein of QoIs which have been found to be controlled by specific QoIs (WO 2013/092224). Despite several commercial QoI fungicides have also been widely used in soybean rust control, the single amino acid residue substitution G143A in the cytochrome b protein conferring resistance to QoI fungicides was not observed.

Instead soybean rust acquired a different genetic mutation in the cytochrome b gene causing a single amino acid substitution F129L which also confers resistance against QoI fungicides. The efficacy of QoI fungicides used against soybean rust conventionally, i.e. pyraclostrobin, azoxystrobin, picoxystrobin, orysastrobin, dimoxystrobin and metominostrobin, has decreased to a level with practical problems for agricultural practice.

Recently, WO 2020/027216 proposed certain strobilurin-type compounds for the use soybean rust containing a single amino acid substitution F129L conferring resistance against QoI fungicides.

Thus, new methods are desirable for controlling pathogen induced diseases in crops comprising plants subjected to pathogens containing a F129L amino acid substitution in the mitochondrial cytochrome b protein conferring resistance to Qo inhibitors. Furthermore, in many cases, in particular at low application rates, the fungicidal activity of the known fungicidal strobilurin compounds is unsatisfactory, especially in case that a high proportion of the fungal pathogens contain a F129L amino acid substitution in the mitochondrial cytochrome b protein conferring resistance to Qo inhibitors. Besides there is an ongoing need for new fungicidally active compounds which are more effective, less toxic and/or environmentally safer. Based on this, it was also an object of the present invention to provide compounds having improved activity and/or a broader activity spectrum against phytopathogenic fungi and/or even further reduced toxicity against non-target organisms such as vertebrates and invertebrates.

The strobilurin-analogue compounds used to combat phytopathogenic fungi containing a F129L amino acid substitution in the mitochondrial cytochrome b protein conferring resistance to Qo inhibitors according to the present invention differ from those described in EP 370629, EP 463488, EP 472300, WO 1990/07493, WO 1992/13830, WO 1992/18487 and WO 2020/027216 inter alia by containing a specific group attached to the central phenyl ring in ortho position to the methyl oxime side chain defined herein as R³. The strobilurin-analogue compounds used to combat phytopathogenic fungi containing a F129L amino acid substitution in the mitochondrial cytochrome b protein conferring resistance to Qo inhibitors according to the present invention differ from trifloxystrobin inter alia described in US 2010/216636 by containing a specific group attached to the central phenyl ring in ortho position to the methyl oxime side chain defined herein as R³ and by a fused partially unsaturated 5- to 6-membered carbo- or heterocycle as defined herein.

Accordingly, the present invention provides novel compounds of formula I

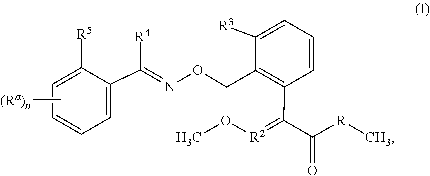

wherein
R¹ is selected from O and NH;
R² is selected from CH and N;
R³ is selected from halogen, CN, C₁-C₄-alkyl, C₂-C₄-alkenyl, C₂-C₄-alkynyl, C₁-C₄-haloalkyl, C₂-C₄-haloalkenyl, C₂-C₄-haloalkynyl, C₃-C₆-cycloalkyl, —O—C₁-C₄-alkyl, —O—C₁-C₄-haloalkyl, —O—C₃-C₆-cycloalkyl, —C₁-C₂-alkyl-C₃-C₆-cycloalkyl, phenyl, 3- to 6-membered heterocycloalkyl and 5- or 6-membered heteroaryl,
   wherein said heterocycloalkyl and heteroaryl besides carbon atoms contain 1, 2 or 3 heteroatoms selected from N, O and S provided that such heterocycloalkyl and heteroaryl cannot contain 2 contiguous atoms selected from O and S,
   wherein said phenyl, heterocycloalkyl and heteroaryl are bound directly or via an oxygen atom or via a C₁-C₂-alkylene linker, and wherein said phenyl and heteroaryl are unsubstituted or substituted by 1, 2 or 3 identical or different substituents selected from halogen, CN, NH₂, NO₂, C₁-C₄-alkyl, C₁-C₄-haloalkyl, —O—C₁-C₄-alkyl and —O—C₁-C₄-haloalkyl;
R⁴ and R⁵, together with the three interjacent carbon atoms, form a partially unsaturated 5- to 6-membered carbo- or heterocycle,
   wherein the heterocycle includes beside carbon atoms 1, 2 or 3 heteroatoms independently selected from N, O and S as ring member atoms provided that such heterocycle cannot contain 2 contiguous atoms selected from O and S; and
   wherein the carbo- or heterocycle is unsubstituted or carries 1, 2 or up to the maximum possible number of identical or different groups R⁴⁵; wherein
   R⁴⁵ is selected from halogen, CN, C₁-C₄-alkyl, C₁-C₄-haloalkyl, phenyl, C₃-C₆-cycloalkyl and —C₁-C₂-alkyl-C₃-C₆-cycloalkyl; wherein it is possible that two R⁴⁵ substituents which are bound to the same carbon atom or to two adjacent carbon atoms form a saturated 3- to 5-membered carbocycle;
      and wherein the cyclic moieties of R⁴⁵ are unsubstituted or carry 1, 2 or 3 identical or different groups R^{45b}:
      R^{45b} is selected from halogen, CN, NH₂, NO₂, C₁-C₄-alkyl, C₁-C₄-haloalkyl, —O—C₁-C₄-alkyl and —O—C₁-C₄-haloalkyl;
R^{a} is selected from halogen, CN, —NR⁵R⁶, C₁-C₄-alkyl, C₂-C₄-alkenyl, C₂-C₄-alkynyl, —O—C₁-C₄-alkyl, —C(═N—O—C₁-C₄-alkyl)-C₁-C₄-alkyl, —C(═O)—C₁-C₄-alkyl, —O—CH₂—C(═N—O—C₁-C₄-alkyl)-C₁-C₄-alkyl, C₃-C₆-cycloalkyl, C₃-C₆-cycloalkenyl, —C₁-C₂-alkyl-C₃-C₆-cycloalkyl, —O—C₃-C₆-cycloalkyl, phenyl, 3- to 6-membered heterocycloalkyl, 3- to 6-membered heterocycloalkenyl and 5- or 6-membered heteroaryl,
   wherein said heterocycloalkyl, heterocycloalkenyl and heteroaryl besides carbon atoms contain 1, 2 or 3 heteroatoms selected from N, O and S provided that such heterocycloalkyl, heterocycloalkenyl and heteroaryl cannot contain 2 contiguous atoms selected from O and S,
   wherein said phenyl, heterocycloalkyl, heterocycloalkenyl and heteroaryl are bound directly or via an oxygen atom or via a C₁-C₂-alkylene linker, and wherein the aliphatic and cyclic moieties of R^{a} are unsubstituted or carry 1, 2, 3, 4 or up to the maximum number of identical or different groups R^{b}:
   R^{b} is selected from halogen, CN, NH₂, NO₂, C₁-C₄-alkyl, C₁-C₄-haloalkyl, —O—C₁-C₄-alkyl and —O—C₁-C₄-haloalkyl;
   R⁵, R⁶ are independently of each other selected from the group consisting of H, C₁-C₆-alkyl, C₁-C₆-haloalkyl and C₂-C₄-alkynyl;
n is an integer selected from 0, 1, 2, 3 and 4;
   and in form or stereoisomers and tautomers thereof, and the N-oxides and the agriculturally acceptable salts thereof.

Although the present invention will be described with respect to particular embodiments, this description is not to be construed in a limiting sense.

Before describing in detail exemplary embodiments of the present invention, definitions important for understanding the present invention are given. As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising of”.

Unless otherwise indicated, the following definitions are set forth to illustrate and define the meaning and scope of the various terms used to describe the invention herein and the appended claims. These definitions should not be interpreted in the literal sense as they are not intended to be general definitions and are relevant only for this application.

The term “compounds I” refers to compounds of formula I. Likewise, this terminology applies to all sub-formulae, e. g. “compounds 1.2” refers to compounds of formula I.2 or “compounds V” refers to compounds of formula V, etc.

The term “independently” when used in the context of selection of substituents for a variable, it means that where more than one substituent is selected from a number of possible substituents, those substituents may be the same or different.

The organic moieties or groups mentioned in the above definitions of the variables are collective terms for individual listings of the individual group members. The term “Cᵥ-C_{w}” indicates the number of carbon atom possible in each case.

The term “halogen” refers to fluorine, chlorine, bromine and iodine.

The term “C₁-C₄-alkyl” refers to a straight-chained or branched saturated hydrocarbon group having 1 to 4 carbon atoms, for example, methyl (CH₃), ethyl (C₂H₅), propyl, 1-methylethyl (isopropyl), butyl, 1-methylpropyl, 2-methylpropyl, 1,1-dimethylethyl.

The term “C₂-C₄-alkenyl” refers to a straight-chain or branched unsaturated hydrocarbon radical having 2 to 4 carbon atoms and a double bond in any position such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₄-alkynyl” refers to a straight-chain or branched unsaturated hydrocarbon radical having 2 to 4 carbon atoms and containing at least one triple bond such as ethynyl, prop-1-ynyl, prop-2-ynyl, but-1-ynyl, but-2-ynyl, but-3-ynyl, 1-methyl-prop-2-ynyl.

The term “C₁-C₄-haloalkyl” refers to a straight-chained or branched alkyl group having 1 to 4 carbon atoms wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F5, CF₂—C₂F5, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl.

The term “—O—C₁-C₄-alkyl” refers to a straight-chain or branched alkyl group having 1 to 4 carbon atoms which is bonded via an oxygen, at any position in the alkyl group, e.g. OCH₃, OCH₂CH₃, O(CH₂)₂CH₃, 1-methylethoxy, O(CH₂)₃CH₃, 1-methylpropoxy, 2-methylpropoxy or 1,1-dimethylethoxy.

The term “C₃-C₆-cycloalkyl” refers to monocyclic saturated hydrocarbon radicals having 3 to 6 carbon ring members, such as cyclopropyl (C₃H₅), cyclobutyl, cyclopentyl or cyclohexyl. The term “C₃-C₆-cycloalkenyl” refers to monocyclic saturated hydrocarbon radicals having 3 to 6 carbon ring members and one or more double bonds.

The term “3- to 6-membered heterocycloalkyl” refers to 3- to 6-membered monocyclic saturated ring system having besides carbon atoms one or more heteroatoms, such as O, N, S as ring members. The term “C₃-C₆-membered heterocycloalkenyl” refers to 3- to 6-membered monocyclic ring system having besides carbon atoms one or more heteroatoms, such as O, N and S as ring members, and one or more double bonds.

The term “—C₁-C₄-alkyl-C₃-C₅-cycloalkyl” refers to alkyl having 1 to 4 carbon atoms (as defined above), wherein one hydrogen atom of the alkyl radical is replaced by a cycloalkyl radical having 3 to 6 carbon atoms.

The term “phenyl” refers to C₆H₅.

The term “5- or 6-membered heteroaryl” which contains 1, 2, 3 or 4 heteroatoms from the group consisting of O, N and S, is to be understood as meaning aromatic heterocycles having 5 or 6 ring atoms. Examples include:
5-membered heteroaryl which in addition to carbon atoms, e.g. contain 1, 2 or 3 N atoms and/or one sulfur and/or one oxygen atom: for example 2-thienyl, 3-thienyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl which, in addition to carbon atoms, e.g. contain 1, 2, 3 or 4 N atoms as ring members, e.g. 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

The term “C₁-C₂-alkylene linker” means a divalent alkyl group such as —CH₂— or —CH₂—CH₂— that is bound at one end to the core structure of formula I and at the other end to the particular substituent.

As used herein, the “compounds”, in particular “compounds I” include all the stereoisomeric and tautomeric forms as well as resonance or canonical structures and mixtures thereof in all ratios, prodrugs, isotopic forms, their agriculturally acceptable salts, N-oxides and S-oxides thereof.

The term “resonance or canonical structures” is a general term used to describe the result of differences in bonding in certain molecules or ions by the combination of several contributing structures used in particular to describe delocalized electrons where bonding cannot be expressed by one single Lewis structure.

The term “stereoisomer” is a general term used for all isomers of individual compounds that differ only in the orientation of their atoms in space. The term stereoisomer includes mirror image isomers (enantiomers), mixtures of mirror image isomers (racemates, racemic mixtures), geometric (cis/trans or E/Z) isomers, and isomers of compounds with more than one chiral center that are not mirror images of one another (diastereoisomers). The term “tautomer” refers to the coexistence of two (or more) compounds that differ from each other only in the position of one (or more) mobile atoms and in electron distribution, for example, keto-enol tautomers. The term “agriculturally acceptable salts” as used herein, includes salts of the active compounds which are prepared with acids or bases, depending on the particular substituents found on the compounds described herein. “N-oxide” refers to the oxide of the nitrogen atom of a nitrogen-containing heteroaryl or heterocycle. N-oxide can be formed in the presence of an oxidizing agent for example peroxide such as m-chloro-perbenzoic acid or hydrogen peroxide. N-oxide refers to an amine oxide, also known as amine-N-oxide, and is a chemical compound that contains N→O bond.

In respect of the variables, the embodiments of the intermediates correspond to the embodiments of the compounds 1.

Preference is given to those compounds I and where applicable also to compounds of all sub-formulae provided herein, e. g. formulae I.1 and I.2, and to the intermediates such as compounds II, III, IV and V, wherein the substituents and variables (such as n, R¹, R², R³, R⁴, R⁵, R⁶, R^{a}, and R^{b}) have independently of each other or more preferably in combination (any possible combination of 2 or more substituents as defined herein) the following meanings:

Preference is also given to the uses, methods, mixtures and compositions, wherein the definitions (such as phytopathogenic fungi, treatments, crops, compounds 1l, further active ingredients, solvents, solid carriers) have independently of each other or more preferably in combination the following meanings and even more preferably in combination (any possible combination of 2 or more definitions as provided herein) with the preferred meanings of compounds I herein:

One embodiment of the invention relates to compounds I, wherein R¹ is selected from O and NH; and R² is selected from CH and N, provided that R² is N in case R¹ is NH. More preferably R¹ is NH. In particular, R¹ is NH and R² is N.

According to another embodiment, R³ is selected from CN, C₁-C₄-alkyl, C₂-C₄-alkenyl, C₁-C₄-haloalkyl, C₂-C₄-haloalkenyl, C₃-C₆-cycloalkyl, —O—C₁-C₄-alkyl, —O—C₁-C₄-haloalkyl, —C₁-C₂-alkyl-C₃-C₆-cycloalkyl and 3- to 6-membered heterocycloalkyl; more preferably from is selected from CN, C₁-C₄-alkyl, C₂-C₄-alkenyl, C₂-C₄-alkynyl, C₁-C₄-haloalkyl, C₃-C₄-cycloalkyl, —O—C₁-C₄-alkyl, —O—C₁-C₄-haloalkyl and 3- to 4-membered heterocycloalkyl, wherein said heterocycloalkyl besides carbon atoms contain 1 or 2 heteroatoms selected from N, O and S, and wherein said heterocycloalkyl is bound directly or via an oxygen atom or via a C₁-C₂-alkylene linker; even more preferably from C₁-C₂-alkyl, C₂-alkenyl, C₁-C₂-haloalkyl, —O—C₁-C₂-alkyl, —O—C₁-C₂-haloalkyl, C₃-C₄-cycloalkyl, —C₁-C₂-alkyl-C₃-C₄-cycloalkyl, and 3- to 4-membered heterocycloalkyl; further more preferably form C₁-C₂-alkyl, C₁-C₂-haloalkyl, C₃-C₄-cycloalkyl, —O—C₁-C₂-alkyl and —O—C₁-C₂-haloalkyl; particularly preferred from methyl and C₁-C₂-haloalkyl, in particular methyl.

According to one embodiment, R⁴ and R⁵, together with the three interjacent carbon atoms, form a partially unsaturated 5- to 6-membered carbo- or heterocycle, wherein the heterocycle includes beside carbon atoms 1 or 2 heteroatoms independently selected from N, O and S as ring member atoms provided that such heterocycle cannot contain 2 contiguous atoms selected from O and S. Preferably, R⁴ and R⁵, together with the three interjacent carbon atoms, form a partially unsaturated 5- to 6-membered carbocycle or partially unsaturated 6-membered heterocycle wherein the heterocycle includes beside carbon atoms 1 heteroatom selected from N, O and S as ring member atoms; even more preferably, R⁴ and R⁵, together with the three interjacent carbon atoms, form a cyclopentene, cyclopentadiene or cyclohexene ring.

Said partially unsaturated 5- to 6-membered carbocycle may be for example a cyclopentene ring (resulting together with the fused phenyl in an indane bicyclic ring system), a cyclopentadiene ring (resulting together with the fused phenyl in an 1H-indene bicyclic ring system) or a cyclohexene ring (resulting together with the condensed phenyl ring in a tetralin bicyclic ring system).

Said partially unsaturated 5- to 6-membered heterocycle may be for example a 2,3-dihydrofuran ring (resulting together with the fused phenyl in an 2,3-dihydrobenzofuran bicyclic ring system), a 3,4-dihydro-2H-pyran ring (resulting together with the fused phenyl in a chromane bicyclic ring system), a furan ring (resulting together with the fused phenyl ring in a benzofuran bicyclic ring system) or a 2,3-dihydro-1H-pyrrole ring (resulting together with the fused phenyl ring in a indoline bicyclic ring system).

According to the abovementioned embodiments for R⁴ and R⁵, said carbo- or heterocycle is preferably unsubstituted or carries 1, 2, 3 or 4 identical or different groups R⁴⁵; wherein R⁴⁵ is selected from halogen, C₁-C₄-alkyl, C₁-C₄-haloalkyl, C₃-C₆-cycloalkyl, phenyl and —C₁-C₂-alkyl-C₃-C₅-cycloalkyl; wherein it is possible that two R⁴⁵ substituents which are bound to the same carbon atom or to two adjacent carbon atoms form a saturated 3- to 5-membered carbocycle. More preferably, said carbo- or heterocycle is unsubstituted or carries 1 or 2 identical or different groups R⁴⁵; wherein R⁴⁵ is selected from halogen, C₁-C₄-alkyl, C₁-C₄-haloalkyl and phenyl, wherein it is possible that two R⁴⁵ substituents which are bound to the same carbon atom or to two adjacent carbon atoms form a saturated 3- to 5-membered carbocycle. Even more preferably, said carbo- or heterocycle is unsubstituted or carries 1 or 2 identical or different groups R⁴⁵; wherein R⁴⁵ is selected from halogen, C₁-C₄-alkyl, C₁-C₄-haloalkyl and phenyl, wherein it is possible that two R⁴⁵ substituents which are bound to the same carbon atom form a cyclopropyl ring, and wherein the cyclic moieties of R⁴⁵ are unsubstituted or carry 1, 2 or 3 identical or different groups R^{45b} wherein R^{45b} is preferably selected from halogen, C₁-C₄-alkyl and C₁-C₄-haloalkyl. Likewise preferred, said partially unsaturated carbo- or heterocycle formed by R⁴ and R⁵ is unsubstituted.

According to a further embodiment, n is 1, 2, 3 or 4; more preferably n is 1, 2 or 3, even more preferably n is 1 or 2; in particular n is 1.

According to a further embodiment, n is 0, 1, 2 or 3, more preferably 0, 1 or 2, in particular 0.

According to a further embodiment, R^{a} is selected from CN, C₁-C₄-alkyl, C₂-C₄-alkenyl, C₂-C₄-alkynyl, —O—C₁-C₄-alkyl, —C(═O)—C₁-C₄-alkyl, —C(═N—O—C₁-C₄-alkyl)-C₁-C₄-alkyl, —O—CH₂—(═N—O—C₁-C₄-alkyl)-C₁-C₄-alkyl, —C(═N—O—C₁-C₄-alkyl)-C(═O—NH—C₁-C₄-alkyl), C₃-C₆-cycloalkyl, C₃-C₆-cycloalkenyl, —C₁-C₂-alkyl-C₃-C₆-cycloalkyl, —O—C₃-C₆-cycloalkyl, phenyl, 3- to 5-membered heterocycloalkyl, 3- to 5-membered heterocycloalkenyl and 5- or 6-membered heteroaryl, wherein said heterocycloalkyl, hetercycloalkenyl and heteroaryl besides carbon atoms contain 1, 2 or 3 heteroatoms selected from N, O and S provided that such heterocycloalkyl, heterocycloalkenyl and heteroaryl cannot contain 2 contiguous atoms selected from O and S, wherein said phenyl, heterocycloalkyl, hetercycloalkenyl and heteroaryl are bound directly or via an oxygen atom or via a C₁-C₂-alkylene linker, and wherein the aliphatic and cyclic moieties of R^{a} are unsubstituted or carry 1, 2, or 3 of identical or different groups R^{b} which independently of one another are selected from halogen, CN, NH₂, NO₂, C₁-C₂-alkyl and C₁-C₂-haloalkyl.

More preferably, R^{a} is selected from CN, C₁-C₄-alkyl, C₂-C₄-alkenyl, C₂-C₄-alkynyl, —O—C₁-C₄-alkyl, —C(═O)—C₁-C₂-alkyl, —C(═N—O—C₁-C₂-alkyl)-C₁-C₂-alkyl, —O—CH₂—C(═N—O—C₁-C₂-alkyl)-C₁-C₂-alkyl, —C(═N—O—C₁-C₂-alkyl)-C(═O—NH—C₁-C₂-alkyl), C₃-C₄-cycloalkyl, C₃-C₄-cycloalkenyl, —C₁-C₂-alkyl-C₃-C₄-cycloalkyl, —O—C₃-C₄-cycloalkyl, phenyl, 3- to 5-membered heterocycloalkyl and 5- or 6-membered heteroaryl, wherein said heterocycloalkyl and heteroaryl besides carbon atoms contain 1 or 2 heteroatoms selected from N, O and S provided that such heterocycloalkyl and heteroaryl cannot contain 2 contiguous atoms selected from O and S, wherein said phenyl, heterocycloalkyl and heteroaryl are bound directly or via an oxygen atom or via a methylene linker, and wherein the aliphatic or cyclic moieties of R^{a} are unsubstituted or carry 1, 2, or 3 of identical or different groups R^{b} which independently of one another are selected from halogen, CN, C₁-C₂-alkyl and C₁-C₂-haloalkyl.

Even more preferably R^{a} is selected from C₁-C₃-alkyl, C₂-C₃-alkenyl, C₂-C₃-alkynyl, —O—C₁-C₃-alkyl, —C(═O)—C₁-C₂-alkyl, —C(═N—O—C₁-C₂-alkyl)-C₁-C₂-alkyl, C₃-C₄-cycloalkyl, —C₁-C₂-alkyl-C₃-C₄-cycloalkyl, —O—C₃-C₄-cycloalkyl, phenyl, 3- to 5-membered heterocycloalkyl and 5- or 6-membered heteroaryl, wherein said heterocycloalkyl and heteroaryl besides carbon atoms contain 1 or 2 heteroatoms selected from N, O and S provided that such heterocycloalkyl and heteroaryl cannot contain 2 contiguous atoms selected from O and S, wherein said phenyl, heterocycloalkyl and heteroaryl are bound directly or via an oxygen atom or via a methylene linker, and wherein the aliphatic and cyclic moieties of R^{a} are unsubstituted or carry 1, 2 or 3 of identical or different groups R^{b} which independently of one another are selected from halogen, CN, methyl and C₁-haloalkyl.

Particularly preferred R^{a} are selected from halogen, C₁-C₄-alkyl, C₂-C₃-alkenyl, C₂-C₃-alkynyl, —O—C₁-C₄-alkyl, —C(═N—O—C₁-C₂-alkyl)-C₁-C₂-alkyl and phenyl, wherein the aliphatic or cyclic moieties of R^{a} are unsubstituted or carry 1, 2 or 3 of identical or different groups R^{b} which independently of one another are selected from halogen, CN, methyl and C₁-haloalkyl.

According to a further embodiment, R⁵, R⁶ are independently of each other preferably selected from the group consisting of H, C₁-C₄-alkyl, C₁-C₄-haloalkyl and C₂-C₄-alkynyl, more preferably from H and C₁-C₄-alkyl.

According to a further preferred embodiment, the present invention relates to compounds of formula I wherein:
R¹ is selected from 0 and NH; and
R² is selected from CH and N, provided that R² is N in case R¹ is NH;
R³ is selected from halogen, C₁-C₄-alkyl, C₁-C₄-haloalkyl;
R⁴ and R⁵, together with the three interjacent carbon atoms, form a partially unsaturated 5- to 6-membered carbocycle, wherein said carbocycle is unsubstituted or carries 1 or 2 identical or different groups R⁴⁵, wherein R⁴⁵ is selected from halogen, C₁-C₄-alkyl, C₁-C₄-haloalkyl, phenyl and C₃-C₆-cycloalkyl, wherein it is possible that two R⁴⁵ substituents which are bound to the same carbon atom form a cyclopropyl ring; and wherein the cyclic moieties of R⁴⁵ are unsubstituted or carry 1, 2 or 3 identical or different groups R^{45b}:
   R^{45b} is selected from halogen, C₁-C₄-alkyl and C₁-C₄-haloalkyl;
R^{a} is selected from halogen, CN, —NR⁵R⁶, C₁-C₄-alkyl, C₂-C₄-alkenyl, C₂-C₄-alkynyl, —O—C₁-C₄-alkyl, —C(═N—O—C₁-C₄-alkyl)-C₁-C₄-alkyl, —C(═O)—C₁-C₄-alkyl, —O—CH₂—C(═N—O—C₁-C₄-alkyl)-C₁-C₄-alkyl, C₃-C₆-cycloalkyl, C₃-C₆-cycloalkenyl, —C₁-C₂-alkyl-C₃-C₆-cycloalkyl, —O—C₃-C₆-cycloalkyl, phenyl, 3- to 6-membered heterocycloalkyl, 3- to 6-membered hetero cycloalkenyl and 5- or 6-membered heteroaryl,
   wherein said heterocycloalkyl, heterocycloalkenyl and heteroaryl besides carbon atoms contain 1, 2 or 3 heteroatoms selected from N, O and S provided that such heterocycloalkyl, heterocycloalkenyl and heteroaryl cannot contain 2 contiguous atoms selected from O and S,
   wherein said phenyl, heterocycloalkyl, heterocycloalkenyl and heteroaryl are bound directly or via an oxygen atom or via a C₁-C₂-alkylene linker,
   and wherein the aliphatic and cyclic moieties of R^{a} are unsubstituted or carry 1, 2, 3, 4 or up to the maximum number of identical or different groups R^{b}:
   R^{b} is selected from halogen, CN, NH₂, NO₂, C₁-C₄-alkyl, C₁-C₄-haloalkyl, —O—C₁-C₄-alkyl and —O—C₁-C₄-haloalkyl;
   R⁵, R⁶ are independently of each other selected from the group consisting of H, C₁-C₆-alkyl and C₂-C₄-alkynyl;
n is an integer selected from 0, 1, 2 and 3;
   and in form or stereoisomers and tautomers thereof, and the N-oxides and the agriculturally acceptable salts thereof.

One embodiment of the invention relates to preferred compounds I, wherein R¹ is selected from O and NH; and R² is selected from CH and N, provided that R² is N in case R¹ is NH. More preferably R¹ is NH. In particular, R¹ is NH and R² is N. Another embodiment of the invention relates to preferred compounds I, wherein R¹ is selected from O and NH; and R² is selected from CH and N, provided that R² is CH in case R¹ is O. More preferably, R² is N and R¹ is NH or R² is CH and R¹ is O.

According to a further embodiment, R¹ is O and R² is N, which compounds are of formula I.1:

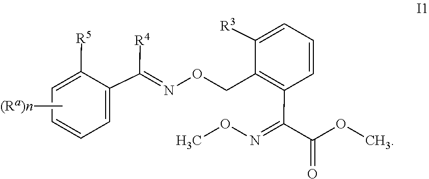

According to a further embodiment, R¹ is O and R² is CH, which compounds are of formula I.2:

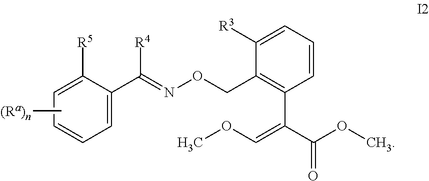

According to a further embodiment, R¹ is NH and R² is N, which compounds are of formula I.3:

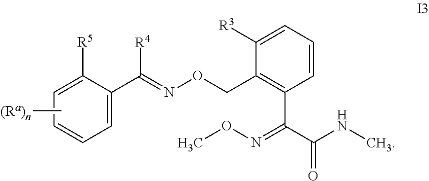

Preferably, R³ of compounds I is one of the following radicals 3-1 to 3-8:

| No. | R³ |
|---|---|
| 3-1 | CH₃ |
| 3-2 | OCH₃ |
| 3-3 | CHF₂ |
| 3-4 | C₃H₅ |
| 3-5 | CH═CH₂ |
| 3-6 | CH₂CH═C(CH₃)₂ |
| 3-7 | CF₃ |
| 3-8 | C(═NOCH₃)CH₃ |

Even more preferably R³ is CH₃, OCH₃, CF₃, CHF₂ or C₃H₅, in particular CH₃.

Particularly preferred embodiments of the invention relate to compounds I, wherein R^{a} is selected of one of the following radicals a-1 to a-18:

| No. | R^{a} |
|---|---|
| a-1 | F |
| a-2 | Cl |
| a-3 | Br |
| a-4 | CH₃ |
| a-5 | CHF₂ |
| a-6 | CF₃ |
| a-7 | OCH₃ |
| a-8 | OCHF₂ |
| a-9 | OCF₃ |
| a-10 | C₂H₅ |
| a-11 | CH₂CF₃ |
| a-12 | CH═CH₂ |
| a-13 | C₆H₅ |
| a-14 | C≡CH |
| a-15 | C≡CCH₃ |
| a-16 | C₃H₅ |
| a-17 | C(═NOCH₃)CH₃ |
| a-18 | CN |

Preferred embodiments of the invention relate to compounds I wherein n is 1 and R^{a} is bound to the phenyl ring in meta-position to the carbon atom bearing R⁵ which are represented by formula IX:

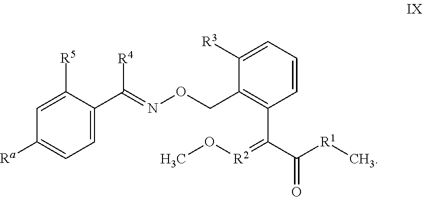

Preferred embodiments of the invention relate to compounds I wherein n is 1 and R^{a} is bound to the phenyl ring in para-position to the carbon atom bearing R⁴ which are represented by formula I.Y:

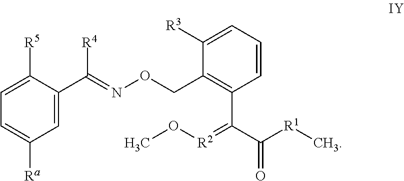

Preferred embodiments of the invention relate to compounds I wherein n is 1 and R^{a} is bound to the phenyl ring in ortho-position to the carbon atom bearing R⁵ which are represented by formula I.Z:

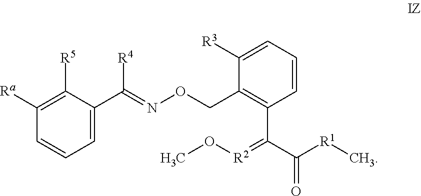

Preferred embodiments of the invention relate to compounds I which are represented by formula I.A, wherein m is 0, 1 or 2:

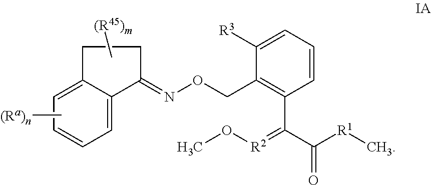

Preferred are also compounds I which are represented by formula I.B, wherein m is 0, 1 or 2:

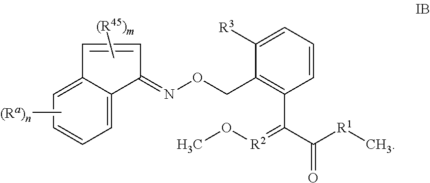

Preferred are also compounds I which are represented by formula I.C, wherein m is 0, 1 or 2:

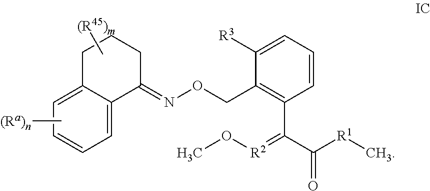

Preferred are also compounds I which are represented by formula I.D, wherein m is 0, 1 or 2:

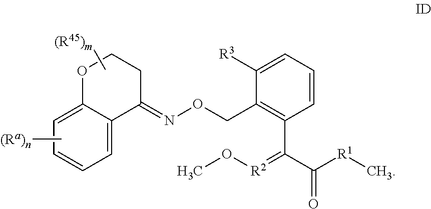

Particularly preferred embodiments of the invention relate to compounds I which are represented by formula I.A.1, wherein m is 0, 1 or 2:

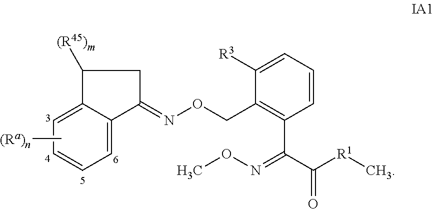

Particularly preferred embodiments of the invention relate to compounds I which are represented by formula I.A.1, wherein m is 0, 1 or 2:

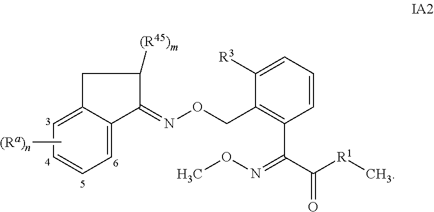

Particularly preferred embodiments of the invention relate to compounds I which are represented by formula I.B.1:

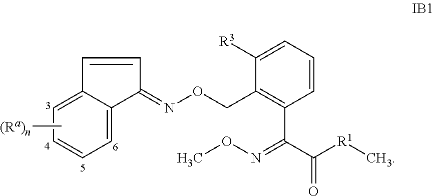

Particularly preferred embodiments of the invention relate to compounds I which are represented by formula I.C.1:

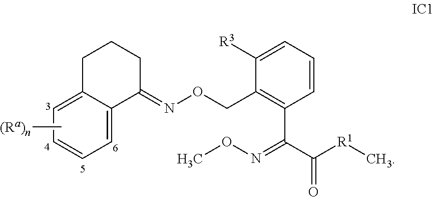

Particularly preferred embodiments of the invention relate to compounds I which are represented by formula I.C.1:

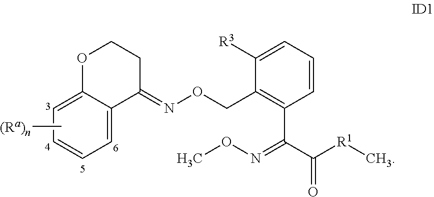

In an embodiment, compounds I are of formula I.A.1, wherein R¹ is N, and n, R^{a}, R⁴⁵, m and R³ are as per any row of Table A below, which compounds are named I.A.1 N-A-1 to I.A.1 N-369.

In another embodiment, compounds I are of formula I.A.1, wherein R¹ is O, and n, R^{a}, R⁴⁵, m and R³ are as per any row of Table A below, which compounds are named I.A.1O-1 to I.A.1O-369.

In another embodiment, compounds I are of formula I.A.2, wherein R¹ is N, and n, R^{a}, R⁴⁵, m and R³ are as per any row of Table A below, which compounds are named I.A.2N-1 to I.A.2N-369.

In another embodiment, compounds I are of formula I.A.2, wherein R¹ is O, and n, R^{a}, R⁴⁵, m and R³ are as per any row of Table A below, which compounds are named I.A.20-1 to I.A.20-369.

In another embodiment, compounds I are of formula I.B.1, wherein R¹ is N, and n, R^{a} and R³ are as per any of the rows A-1 to A-41 of Table A below, which compounds are named I.B.1N-1 to I.B.1N-41.

In another embodiment, compounds I are of formula I.B.1, wherein R¹ is O, and n, R^{a} and R³ are as per any of the rows A-1 to A-41 of Table A below, which compounds are named I.B.1O-1 to I.B.1O-41.

In another embodiment, compounds I are of formula I.C.1, wherein R¹ is N, and n, R^{a} and R³ are as per any of the rows A-1 to A-41 of Table A below, which compounds are named I.C.1 N-1 to I.C.1N-41.

In another embodiment, compounds I are of formula I.C.1, wherein R¹ is O, and n, R^{a} and R³ are as per any of the rows A-1 to A-41 of Table A below, which compounds are named I.C.1O-1 to I.C.1O-41.

In another embodiment, compounds I are of formula I.D.1, wherein R¹ is N, and n, R^{a} and R³ are as per any of the rows A-1 to A-41 of Table A below, which compounds are named I.D.1 N-1 to I.D.1N-41.

In another embodiment, compounds I are of formula I.D.1, wherein R¹ is O, and n, R^{a} and R³ are as per any of the rows A-1 to A-41 of Table A below, which compounds are named I.D.1O-1 to I.D.1N-41.

TABLE A

| No. | (Rª)ₙ | R⁴⁵ | m | R³ |
|---|---|---|---|---|
| A-1 | — | — | 0 | CH₃ |
| A-2 | 3-F * | — | 0 | CH₃ |
| A-3 | 3-Cl | — | 0 | CH₃ |
| A-4 | 3-Br | — | 0 | CH₃ |
| A-5 | 3-CH₃ | — | 0 | CH₃ |
| A-6 | 3-CHF₂ | — | 0 | CH₃ |
| A-7 | 3-CF₃ | — | 0 | CH₃ |
| A-8 | 3-OCH₃ | — | 0 | CH₃ |
| A-9 | 3-OCHF₂ | — | 0 | CH₃ |
| A-10 | 3-OCF₃ | — | 0 | CH₃ |
| A-11 | 3-C₂H₅ | — | 0 | CH₃ |
| A-12 | 4-F | — | 0 | CH₃ |
| A-13 | 4-Cl | — | 0 | CH₃ |
| A-14 | 4-Br | — | 0 | CH₃ |
| A-15 | 4-CH₃ | — | 0 | CH₃ |
| A-16 | 4-CHF₂ | — | 0 | CH₃ |
| A-17 | 4-CF₃ | — | 0 | CH₃ |
| A-18 | 4-OCH₃ | — | 0 | CH₃ |
| A-19 | 4-OCHF₂ | — | 0 | CH₃ |
| A-20 | 4-OCF₃ | — | 0 | CH₃ |
| A-21 | 4-C₂H₅ | — | 0 | CH₃ |
| A-22 | 5-F | — | 0 | CH₃ |
| A-23 | 5-Cl | — | 0 | CH₃ |
| A-24 | 5-Br | — | 0 | CH₃ |
| A-25 | 5-CH₃ | — | 0 | CH₃ |
| A-26 | 5-CHF₂ | — | 0 | CH₃ |
| A-27 | 5-CF₃ | — | 0 | CH₃ |
| A-28 | 5-OCH₃ | — | 0 | CH₃ |
| A-29 | 5-OCHF₂ | — | 0 | CH₃ |
| A-30 | 5-OCF₃ | — | 0 | CH₃ |
| A-31 | 5-C₂H₅ | — | 0 | CH₃ |
| A-32 | 6-F | — | 0 | CH₃ |
| A-33 | 6-Cl | — | 0 | CH₃ |
| A-34 | 6-Br | — | 0 | CH₃ |
| A-35 | 6-CH₃ | — | 0 | CH₃ |
| A-36 | 6-CHF₂ | — | 0 | CH₃ |
| A-37 | 6-CF₃ | — | 0 | CH₃ |
| A-38 | 6-OCH₃ | — | 0 | CH₃ |
| A-39 | 6-OCHF₂ | — | 0 | CH₃ |
| A-40 | 6-OCF₃ | — | 0 | CH₃ |
| A-41 | 6-C₂H₅ | — | 0 | CH₃ |
| A-42 | — | F | 1 | CH₃ |
| A-43 | 3-F | F | 1 | CH₃ |
| A-44 | 3-Cl | F | 1 | CH₃ |
| A-45 | 3-Br | F | 1 | CH₃ |
| A-46 | 3-CH₃ | F | 1 | CH₃ |
| A-47 | 3-CHF₂ | F | 1 | CH₃ |
| A-48 | 3-CF₃ | F | 1 | CH₃ |
| A-49 | 3-OCH₃ | F | 1 | CH₃ |
| A-50 | 3-OCHF₂ | F | 1 | CH₃ |
| A-51 | 3-OCF₃ | F | 1 | CH₃ |
| A-52 | 3-C₂H₅ | F | 1 | CH₃ |
| A-53 | 4-F | F | 1 | CH₃ |
| A-54 | 4-Cl | F | 1 | CH₃ |
| A-55 | 4-Br | F | 1 | CH₃ |
| A-56 | 4-CH₃ | F | 1 | CH₃ |
| A-57 | 4-CHF₂ | F | 1 | CH₃ |
| A-58 | 4-CF₃ | F | 1 | CH₃ |
| A-59 | 4-OCH₃ | F | 1 | CH₃ |
| A-60 | 4-OCHF₂ | F | 1 | CH₃ |
| A-61 | 4-OCF₃ | F | 1 | CH₃ |
| A-62 | 4-C₂H₅ | F | 1 | CH₃ |
| A-63 | 5-F | F | 1 | CH₃ |
| A-64 | 5-Cl | F | 1 | CH₃ |
| A-65 | 5-Br | F | 1 | CH₃ |
| A-66 | 5-CH₃ | F | 1 | CH₃ |
| A-67 | 5-CHF₂ | F | 1 | CH₃ |
| A-68 | 5-CF₃ | F | 1 | CH₃ |
| A-69 | 5-OCH₃ | F | 1 | CH₃ |
| A-70 | 5-OCHF₂ | F | 1 | CH₃ |
| A-71 | 5-OCF₃ | F | 1 | CH₃ |
| A-72 | 5-C₂H₅ | F | 1 | CH₃ |
| A-73 | 6-F | F | 1 | CH₃ |
| A-74 | 6-Cl | F | 1 | CH₃ |
| A-75 | 6-Br | F | 1 | CH₃ |
| A-76 | 6-CH₃ | F | 1 | CH₃ |
| A-77 | 6-CHF₂ | F | 1 | CH₃ |
| A-78 | 6-CF₃ | F | 1 | CH₃ |
| A-79 | 6-OCH₃ | F | 1 | CH₃ |
| A-80 | 6-OCHF₂ | F | 1 | CH₃ |
| A-81 | 6-OCF₃ | F | 1 | CH₃ |
| A-82 | 6-C₂H₅ | F | 1 | CH₃ |
| A-83 | — | Cl | 1 | CH₃ |
| A-84 | 3-F | Cl | 1 | CH₃ |
| A-85 | 3-Cl | Cl | 1 | CH₃ |
| A-86 | 3-Br | Cl | 1 | CH₃ |
| A-87 | 3-CH₃ | Cl | 1 | CH₃ |
| A-88 | 3-CHF₂ | Cl | 1 | CH₃ |
| A-89 | 3-CF₃ | Cl | 1 | CH₃ |
| A-90 | 3-OCH₃ | Cl | 1 | CH₃ |
| A-91 | 3-OCHF₂ | Cl | 1 | CH₃ |
| A-92 | 3-OCF₃ | Cl | 1 | CH₃ |
| A-93 | 3-C₂H₅ | Cl | 1 | CH₃ |
| A-94 | 4-F | Cl | 1 | CH₃ |
| A-95 | 4-Cl | Cl | 1 | CH₃ |
| A-96 | 4-Br | Cl | 1 | CH₃ |
| A-97 | 4-CH₃ | Cl | 1 | CH₃ |
| A-98 | 4-CHF₂ | Cl | 1 | CH₃ |
| A-99 | 4-CF₃ | Cl | 1 | CH₃ |
| A-100 | 4-OCH₃ | Cl | 1 | CH₃ |
| A-101 | 4-OCHF₂ | Cl | 1 | CH₃ |
| A-102 | 4-OCF₃ | Cl | 1 | CH₃ |
| A-103 | 4-C₂H₅ | Cl | 1 | CH₃ |
| A-104 | 5-F | Cl | 1 | CH₃ |
| A-105 | 5-Cl | Cl | 1 | CH₃ |
| A-106 | 5-Br | Cl | 1 | CH₃ |
| A-107 | 5-CH₃ | Cl | 1 | CH₃ |
| A-108 | 5-CHF₂ | Cl | 1 | CH₃ |
| A-109 | 5-CF₃ | Cl | 1 | CH₃ |
| A-110 | 5-OCH₃ | Cl | 1 | CH₃ |
| A-111 | 5-OCHF₂ | Cl | 1 | CH₃ |
| A-112 | 5-OCF₃ | Cl | 1 | CH₃ |
| A-113 | 5-C₂H₅ | Cl | 1 | CH₃ |
| A-114 | 6-F | Cl | 1 | CH₃ |
| A-115 | 6-Cl | Cl | 1 | CH₃ |
| A-116 | 6-Br | Cl | 1 | CH₃ |
| A-117 | 6-CH₃ | Cl | 1 | CH₃ |
| A-118 | 6-CHF₂ | Cl | 1 | CH₃ |
| A-119 | 6-CF₃ | Cl | 1 | CH₃ |
| A-120 | 6-OCH₃ | Cl | 1 | CH₃ |
| A-121 | 6-OCHF₂ | Cl | 1 | CH₃ |
| A-122 | 6-OCF₃ | Cl | 1 | CH₃ |
| A-123 | 6-C₂H₅ | Cl | 1 | CH₃ |
| A-124 | — | Br | 1 | CH₃ |
| A-125 | 3-F | Br | 1 | CH₃ |
| A-126 | 3-Cl | Br | 1 | CH₃ |
| A-127 | 3-Br | Br | 1 | CH₃ |
| A-128 | 3-CH₃ | Br | 1 | CH₃ |
| A-129 | 3-CHF₂ | Br | 1 | CH₃ |
| A-130 | 3-CF₃ | Br | 1 | CH₃ |
| A-131 | 3-OCH₃ | Br | 1 | CH₃ |
| A-132 | 3-OCHF₂ | Br | 1 | CH₃ |
| A-133 | 3-OCF₃ | Br | 1 | CH₃ |
| A-134 | 3-C₂H₅ | Br | 1 | CH₃ |
| A-135 | 4-F | Br | 1 | CH₃ |
| A-136 | 4-Cl | Br | 1 | CH₃ |
| A-137 | 4-Br | Br | 1 | CH₃ |
| A-138 | 4-CH₃ | Br | 1 | CH₃ |
| A-139 | 4-CHF₂ | Br | 1 | CH₃ |
| A-140 | 4-CF₃ | Br | 1 | CH₃ |
| A-141 | 4-OCH₃ | Br | 1 | CH₃ |
| A-142 | 4-OCHF₂ | Br | 1 | CH₃ |
| A-143 | 4-OCF₃ | Br | 1 | CH₃ |
| A-144 | 4-C₂H₅ | Br | 1 | CH₃ |
| A-145 | 5-F | Br | 1 | CH₃ |
| A-146 | 5-Cl | Br | 1 | CH₃ |
| A-147 | 5-Br | Br | 1 | CH₃ |
| A-148 | 5-CH₃ | Br | 1 | CH₃ |
| A-149 | 5-CHF₂ | Br | 1 | CH₃ |
| A-150 | 5-CF₃ | Br | 1 | CH₃ |
| A-151 | 5-OCH₃ | Br | 1 | CH₃ |
| A-152 | 5-OCHF₂ | Br | 1 | CH₃ |
| A-153 | 5-OCF₃ | Br | 1 | CH₃ |
| A-154 | 5-C₂H₅ | Br | 1 | CH₃ |
| A-155 | 6-F | Br | 1 | CH₃ |
| A-156 | 6-Cl | Br | 1 | CH₃ |
| A-157 | 6-Br | Br | 1 | CH₃ |
| A-158 | 6-CH₃ | Br | 1 | CH₃ |
| A-159 | 6-CHF₂ | Br | 1 | CH₃ |
| A-160 | 6-CF₃ | Br | 1 | CH₃ |
| A-161 | 6-OCH₃ | Br | 1 | CH₃ |
| A-162 | 6-OCHF₂ | Br | 1 | CH₃ |
| A-163 | 6-OCF₃ | Br | 1 | CH₃ |
| A-164 | 6-C₂H₅ | Br | 1 | CH₃ |
| A-165 | — | CH₃ | 1 | CH₃ |
| A-166 | 3-F | CH₃ | 1 | CH₃ |
| A-167 | 3-Cl | CH₃ | 1 | CH₃ |
| A-168 | 3-Br | CH₃ | 1 | CH₃ |
| A-169 | 3-CH₃ | CH₃ | 1 | CH₃ |
| A-170 | 3-CHF₂ | CH₃ | 1 | CH₃ |
| A-171 | 3-CF₃ | CH₃ | 1 | CH₃ |
| A-172 | 3-OCH₃ | CH₃ | 1 | CH₃ |
| A-173 | 3-OCHF₂ | CH₃ | 1 | CH₃ |
| A-174 | 3-OCF₃ | CH₃ | 1 | CH₃ |
| A-175 | 3-C₂H₅ | CH₃ | 1 | CH₃ |
| A-176 | 4-F | CH₃ | 1 | CH₃ |
| A-177 | 4-Cl | CH₃ | 1 | CH₃ |
| A-178 | 4-Br | CH₃ | 1 | CH₃ |
| A-179 | 4-CH₃ | CH₃ | 1 | CH₃ |
| A-180 | 4-CHF₂ | CH₃ | 1 | CH₃ |
| A-181 | 4-CF₃ | CH₃ | 1 | CH₃ |
| A-182 | 4-OCH₃ | CH₃ | 1 | CH₃ |
| A-183 | 4-OCHF₂ | CH₃ | 1 | CH₃ |
| A-184 | 4-OCF₃ | CH₃ | 1 | CH₃ |
| A-185 | 4-C₂H₅ | CH₃ | 1 | CH₃ |
| A-186 | 5-F | CH₃ | 1 | CH₃ |
| A-187 | 5-Cl | CH₃ | 1 | CH₃ |
| A-188 | 5-Br | CH₃ | 1 | CH₃ |
| A-189 | 5-CH₃ | CH₃ | 1 | CH₃ |
| A-190 | 5-CHF₂ | CH₃ | 1 | CH₃ |
| A-191 | 5-CF₃ | CH₃ | 1 | CH₃ |
| A-192 | 5-OCH₃ | CH₃ | 1 | CH₃ |
| A-193 | 5-OCHF₂ | CH₃ | 1 | CH₃ |
| A-194 | 5-OCF₃ | CH₃ | 1 | CH₃ |
| A-195 | 5-C₂H₅ | CH₃ | 1 | CH₃ |
| A-196 | 6-F | CH₃ | 1 | CH₃ |
| A-197 | 6-Cl | CH₃ | 1 | CH₃ |
| A-198 | 6-Br | CH₃ | 1 | CH₃ |
| A-199 | 6-CH₃ | CH₃ | 1 | CH₃ |
| A-200 | 6-CHF₂ | CH₃ | 1 | CH₃ |
| A-201 | 6-CF₃ | CH₃ | 1 | CH₃ |
| A-202 | 6-OCH₃ | CH₃ | 1 | CH₃ |
| A-203 | 6-OCHF₂ | CH₃ | 1 | CH₃ |
| A-204 | 6-OCF₃ | CH₃ | 1 | CH₃ |
| A-205 | 6-C₂H₅ | CH₃ | 1 | CH₃ |
| A-206 | — | CF₃ | 1 | CH₃ |
| A-207 | 3-F | CF₃ | 1 | CH₃ |
| A-208 | 3-Cl | CF₃ | 1 | CH₃ |
| A-209 | 3-Br | CF₃ | 1 | CH₃ |
| A-210 | 3-CH₃ | CF₃ | 1 | CH₃ |
| A-211 | 3-CHF₂ | CF₃ | 1 | CH₃ |
| A-212 | 3-CF₃ | CF₃ | 1 | CH₃ |
| A-213 | 3-OCH₃ | CF₃ | 1 | CH₃ |
| A-214 | 3-OCHF₂ | CF₃ | 1 | CH₃ |
| A-215 | 3-OCF₃ | CF₃ | 1 | CH₃ |
| A-216 | 3-C₂H₅ | CF₃ | 1 | CH₃ |
| A-217 | 4-F | CF₃ | 1 | CH₃ |
| A-218 | 4-Cl | CF₃ | 1 | CH₃ |
| A-219 | 4-Br | CF₃ | 1 | CH₃ |
| A-220 | 4-CH₃ | CF₃ | 1 | CH₃ |
| A-221 | 4-CHF₂ | CF₃ | 1 | CH₃ |
| A-222 | 4-CF₃ | CF₃ | 1 | CH₃ |
| A-223 | 4-OCH₃ | CF₃ | 1 | CH₃ |
| A-224 | 4-OCHF₂ | CF₃ | 1 | CH₃ |
| A-225 | 4-OCF₃ | CF₃ | 1 | CH₃ |
| A-226 | 4-C₂H₅ | CF₃ | 1 | CH₃ |
| A-227 | 5-F | CF₃ | 1 | CH₃ |
| A-228 | 5-Cl | CF₃ | 1 | CH₃ |
| A-229 | 5-Br | CF₃ | 1 | CH₃ |
| A-230 | 5-CH₃ | CF₃ | 1 | CH₃ |
| A-231 | 5-CHF₂ | CF₃ | 1 | CH₃ |
| A-232 | 5-CF₃ | CF₃ | 1 | CH₃ |
| A-233 | 5-OCH₃ | CF₃ | 1 | CH₃ |
| A-234 | 5-OCHF₂ | CF₃ | 1 | CH₃ |
| A-235 | 5-OCF₃ | CF₃ | 1 | CH₃ |
| A-236 | 5-C₂H₅ | CF₃ | 1 | CH₃ |
| A-237 | 6-F | CF₃ | 1 | CH₃ |
| A-238 | 6-Cl | CF₃ | 1 | CH₃ |
| A-239 | 6-Br | CF₃ | 1 | CH₃ |
| A-240 | 6-CH₃ | CF₃ | 1 | CH₃ |
| A-241 | 6-CHF₂ | CF₃ | 1 | CH₃ |
| A-242 | 6-CF₃ | CF₃ | 1 | CH₃ |
| A-243 | 6-OCH₃ | CF₃ | 1 | CH₃ |
| A-244 | 6-OCHF₂ | CF₃ | 1 | CH₃ |
| A-245 | 6-OCF₃ | CF₃ | 1 | CH₃ |
| A-246 | 6-C₂H₅ | CF₃ | 1 | CH₃ |
| A-247 | — | C₂H₅ | 1 | CH₃ |
| A-248 | 3-F | C₂H₅ | 1 | CH₃ |
| A-249 | 3-Cl | C₂H₅ | 1 | CH₃ |
| A-250 | 3-Br | C₂H₅ | 1 | CH₃ |
| A-251 | 3-CH₃ | C₂H₅ | 1 | CH₃ |
| A-252 | 3-CHF₂ | C₂H₅ | 1 | CH₃ |
| A-253 | 3-CF₃ | C₂H₅ | 1 | CH₃ |
| A-254 | 3-OCH₃ | C₂H₅ | 1 | CH₃ |
| A-255 | 3-OCHF₂ | C₂H₅ | 1 | CH₃ |
| A-256 | 3-OCF₃ | C₂H₅ | 1 | CH₃ |
| A-257 | 3-C₂H₅ | C₂H₅ | 1 | CH₃ |
| A-258 | 4-F | C₂H₅ | 1 | CH₃ |
| A-259 | 4-Cl | C₂H₅ | 1 | CH₃ |
| A-260 | 4-Br | C₂H₅ | 1 | CH₃ |
| A-261 | 4-CH₃ | C₂H₅ | 1 | CH₃ |
| A-262 | 4-CHF₂ | C₂H₅ | 1 | CH₃ |
| A-263 | 4-CF₃ | C₂H₅ | 1 | CH₃ |
| A-264 | 4-OCH₃ | C₂H₅ | 1 | CH₃ |
| A-265 | 4-OCHF₂ | C₂H₅ | 1 | CH₃ |
| A-266 | 4-OCF₃ | C₂H₅ | 1 | CH₃ |
| A-267 | 4-C₂H₅ | C₂H₅ | 1 | CH₃ |
| A-268 | 5-F | C₂H₅ | 1 | CH₃ |
| A-269 | 5-Cl | C₂H₅ | 1 | CH₃ |
| A-270 | 5-Br | C₂H₅ | 1 | CH₃ |
| A-271 | 5-CH₃ | C₂H₅ | 1 | CH₃ |
| A-272 | 5-CHF₂ | C₂H₅ | 1 | CH₃ |
| A-273 | 5-CF₃ | C₂H₅ | 1 | CH₃ |
| A-274 | 5-OCH₃ | C₂H₅ | 1 | CH₃ |
| A-275 | 5-OCHF₂ | C₂H₅ | 1 | CH₃ |
| A-276 | 5-OCF₃ | C₂H₅ | 1 | CH₃ |
| A-277 | 5-C₂H₅ | C₂H₅ | 1 | CH₃ |
| A-278 | 6-F | C₂H₅ | 1 | CH₃ |
| A-279 | 6-Cl | C₂H₅ | 1 | CH₃ |
| A-280 | 6-Br | C₂H₅ | 1 | CH₃ |
| A-281 | 6-CH₃ | C₂H₅ | 1 | CH₃ |
| A-282 | 6-CHF₂ | C₂H₅ | 1 | CH₃ |
| A-283 | 6-CF₃ | C₂H₅ | 1 | CH₃ |
| A-284 | 6-OCH₃ | C₂H₅ | 1 | CH₃ |
| A-285 | 6-OCHF₂ | C₂H₅ | 1 | CH₃ |
| A-286 | 6-OCF₃ | C₂H₅ | 1 | CH₃ |
| A-287 | 6-C₂H₅ | C₂H₅ | 1 | CH₃ |
| A-288 | — | C₆H₅ | 1 | CH₃ |
| A-289 | 3-F | C₆H₅ | 1 | CH₃ |
| A-290 | 3-Cl | C₆H₅ | 1 | CH₃ |
| A-291 | 3-Br | C₆H₅ | 1 | CH₃ |
| A-292 | 3-CH₃ | C₆H₅ | 1 | CH₃ |
| A-293 | 3-CHF₂ | C₆H₅ | 1 | CH₃ |
| A-294 | 3-CF₃ | C₆H₅ | 1 | CH₃ |
| A-295 | 3-OCH₃ | C₆H₅ | 1 | CH₃ |
| A-296 | 3-OCHF₂ | C₆H₅ | 1 | CH₃ |
| A-297 | 3-OCF₃ | C₆H₅ | 1 | CH₃ |
| A-298 | 3-C₂H₅ | C₆H₅ | 1 | CH₃ |
| A-299 | 4-F | C₆H₅ | 1 | CH₃ |
| A-300 | 4-Cl | C₆H₅ | 1 | CH₃ |
| A-301 | 4-Br | C₆H₅ | 1 | CH₃ |
| A-302 | 4-CH₃ | C₆H₅ | 1 | CH₃ |
| A-303 | 4-CHF₂ | C₆H₅ | 1 | CH₃ |
| A-304 | 4-CF₃ | C₆H₅ | 1 | CH₃ |
| A-305 | 4-OCH₃ | C₆H₅ | 1 | CH₃ |
| A-306 | 4-OCHF₂ | C₆H₅ | 1 | CH₃ |
| A-307 | 4-OCF₃ | C₆H₅ | 1 | CH₃ |
| A-308 | 4-C₂H₅ | C₆H₅ | 1 | CH₃ |
| A-309 | 5-F | C₆H₅ | 1 | CH₃ |
| A-310 | 5-Cl | C₆H₅ | 1 | CH₃ |
| A-311 | 5-Br | C₆H₅ | 1 | CH₃ |
| A-312 | 5-CH₃ | C₆H₅ | 1 | CH₃ |
| A-313 | 5-CHF₂ | C₆H₅ | 1 | CH₃ |
| A-314 | 5-CF₃ | C₆H₅ | 1 | CH₃ |
| A-315 | 5-OCH₃ | C₆H₅ | 1 | CH₃ |
| A-316 | 5-OCHF₂ | C₆H₅ | 1 | CH₃ |
| A-317 | 5-OCF₃ | C₆H₅ | 1 | CH₃ |
| A-318 | 5-C₂H₅ | C₆H₅ | 1 | CH₃ |
| A-319 | 6-F | C₆H₅ | 1 | CH₃ |
| A-320 | 6-Cl | C₆H₅ | 1 | CH₃ |
| A-321 | 6-Br | C₆H₅ | 1 | CH₃ |
| A-322 | 6-CH₃ | C₆H₅ | 1 | CH₃ |
| A-323 | 6-CHF₂ | C₆H₅ | 1 | CH₃ |
| A-324 | 6-CF₃ | C₆H₅ | 1 | CH₃ |
| A-325 | 6-OCH₃ | C₆H₅ | 1 | CH₃ |
| A-326 | 6-OCHF₂ | C₆H₅ | 1 | CH₃ |
| A-327 | 6-OCF₃ | C₆H₅ | 1 | CH₃ |
| A-328 | 6-C₂H₅ | C₆H₅ | 1 | CH₃ |
| A-329 | — | —CH₂CH₂— | 2 | CH₃ |
| A-330 | 3-F | —CH₂CH₂— | 2 | CH₃ |
| A-331 | 3-Cl | —CH₂CH₂— | 2 | CH₃ |
| A-332 | 3-Br | —CH₂CH₂— | 2 | CH₃ |
| A-333 | 3-CH₃ | —CH₂CH₂— | 2 | CH₃ |
| A-334 | 3-CHF₂ | —CH₂CH₂— | 2 | CH₃ |
| A-335 | 3-CF₃ | —CH₂CH₂— | 2 | CH₃ |
| A-336 | 3-OCH₃ | —CH₂CH₂— | 2 | CH₃ |
| A-337 | 3-OCHF₂ | —CH₂CH₂— | 2 | CH₃ |
| A-338 | 3-OCF₃ | —CH₂CH₂— | 2 | CH₃ |
| A-339 | 3-C₂H₅ | —CH₂CH₂— | 2 | CH₃ |
| A-340 | 4-F | —CH₂CH₂— | 2 | CH₃ |
| A-341 | 4-Cl | —CH₂CH₂— | 2 | CH₃ |
| A-342 | 4-Br | —CH₂CH₂— | 2 | CH₃ |
| A-343 | 4-CH₃ | —CH₂CH₂— | 2 | CH₃ |
| A-344 | 4-CHF₂ | —CH₂CH₂— | 2 | CH₃ |
| A-345 | 4-CF₃ | —CH₂CH₂— | 2 | CH₃ |
| A-346 | 4-OCH₃ | —CH₂CH₂— | 2 | CH₃ |
| A-347 | 4-OCHF₂ | —CH₂CH₂— | 2 | CH₃ |
| A-348 | 4-OCF₃ | —CH₂CH₂— | 2 | CH₃ |
| A-349 | 4-C₂H₅ | —CH₂CH₂— | 2 | CH₃ |
| A-350 | 5-F | —CH₂CH₂— | 2 | CH₃ |
| A-351 | 5-Cl | —CH₂CH₂— | 2 | CH₃ |
| A-352 | 5-Br | —CH₂CH₂— | 2 | CH₃ |
| A-353 | 5-CH₃ | —CH₂CH₂— | 2 | CH₃ |
| A-354 | 5-CHF₂ | —CH₂CH₂— | 2 | CH₃ |
| A-355 | 5-CF₃ | —CH₂CH₂— | 2 | CH₃ |
| A-356 | 5-OCH₃ | —CH₂CH₂— | 2 | CH₃ |
| A-357 | 5-OCHF₂ | —CH₂CH₂— | 2 | CH₃ |
| A-358 | 5-OCF₃ | —CH₂CH₂— | 2 | CH₃ |
| A-359 | 5-C₂H₅ | —CH₂CH₂— | 2 | CH₃ |
| A-360 | 6-F | —CH₂CH₂— | 2 | CH₃ |
| A-361 | 6-Cl | —CH₂CH₂— | 2 | CH₃ |
| A-362 | 6-Br | —CH₂CH₂— | 2 | CH₃ |
| A-363 | 6-CH₃ | —CH₂CH₂— | 2 | CH₃ |
| A-364 | 6-CHF₂ | —CH₂CH₂— | 2 | CH₃ |
| A-365 | 6-CF₃ | —CH₂CH₂— | 2 | CH₃ |
| A-366 | 6-OCH₃ | —CH₂CH₂— | 2 | CH₃ |
| A-367 | 6-OCHF₂ | —CH₂CH₂— | 2 | CH₃ |
| A-368 | 6-OCF₃ | —CH₂CH₂— | 2 | CH₃ |
| A-369 | 6-C₂H₅ | —CH₂CH₂— | 2 | CH₃ |

wherein R⁴⁵ being —CH₂CH₂— and m being 2 means that two R⁴⁵ substituents are attached to the same carbon atom together with said carbon atom form a cyclopropyl ring.
* Position of R^{a} substituent refers to carbon atom number in sketch of respective formula.

### Synthesis

The compounds can be obtained by various routes in analogy to prior art processes known (e.g. EP 463488, WO 2020/027216) and, advantageously, by the synthesis shown in the following schemes 1 to 4 and in the experimental part of this application.

A suitable method to prepare compounds I is illustrated in Scheme 1.

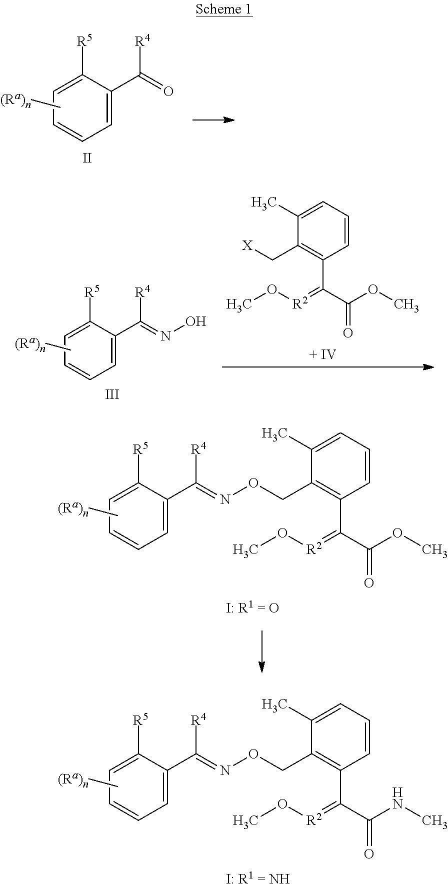

It starts with the conversion of a ketone to the corresponding oxime using hydxroxylamine hydrochloride and a base such as pyridine, sodium hydroxide or sodium acetate in polar solvents such as methanol, methanol-water mixture, or ethanol at reaction temperatures of 60 to 100° C., preferably at about 65° C. In cases where a E/Z mixture was obtained, the isomers could be separated by purification techniques known in art (e.g. column chromatography, crystallization, distillation etc.). Then, coupling with the intermediate IV, wherein X is a leaving group such as halogen, toluene- and methanesulfonates, preferably X is Cl or Br, is carried out under basic conditions using e.g. sodium hydride, cesium carbonate or potassium carbonate as a base and using an organic solvent such as dimethyl formamide (DMF) or acetonitrile, preferably cesium carbonate as base and acetonitrile as solvent at room temperature (RT) of about 24° C. The ester compound I wherein R¹ is O can be converted to the amide of formula I wherein R¹ is NH by reaction with methyl amine (preferably 40% aq. solution) using tetrahydrofuran (THF) as solvent at RT.

Another general method to prepare the compounds I is depicted in Scheme 2.

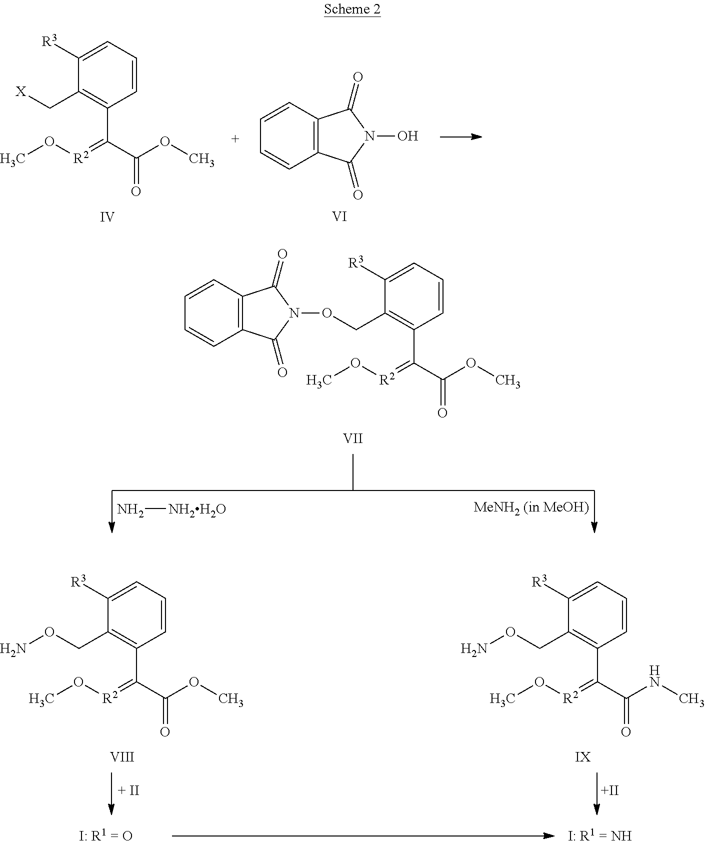

Intermediate IV is reacted with N-hydroxysuccimide VI, using a base such as triethylamine in DMF. The reaction temperature is usually 50 to 70° C. preferably about 70° C. Conversion to the corresponding O-benzylhydroxyl amine, intermediate VIII, was achieved through removal of the phthalimide group, preferably using hydrazine hydrate in methanol as solvent at 25° C. Alternatively, removal of the phthalimide group using methyl amine in methanol as solvent at 25° C. can provide intermediate IX. Intermediate VIII and intermediate IX, respectively can be condensed with ketones using acetic acid or pyridine in methanol as solvent at temperature of 50 to 65° C. Alternatively, the condensation could also carried out with titanium (IV) ethoxide (Ti(OEt)₄) using THF as solvent at about 70° C. The desired product is usually accompanied by an undesired isomer, which can be removed e.g by column chromatography, crystallization.

A general method for preparation of intermediate IV is shown in Scheme 3.

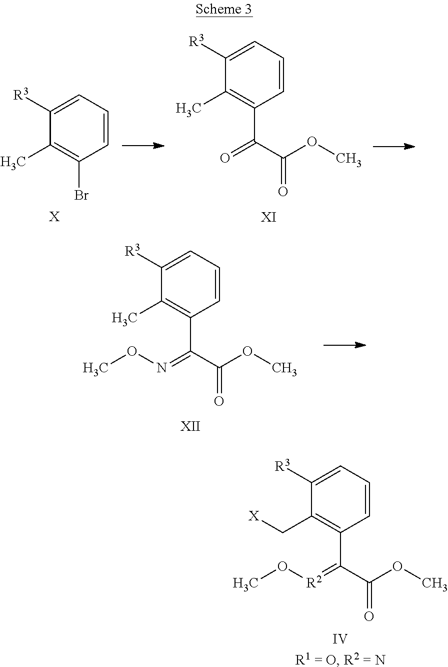

Compound XI could be obtained from X by lithium-halogen exchange or by generating Grignard reagent and further reaction with dimethyl oxalate or chloromethyl oxalate in presence of a solvent. The preferred solvent is THF, 2-methyl-THF and the temperature can be between −70 to −78° C. Conversion of intermediate XI to intermediate XII can be achieved using N-methylhydroxylamine hydrochloride and a base such as pyridine or sodium acetate in polar solvents such as methanol. The reaction temperature is preferably about 65° C. An E/Z mixture is usually obtained, the isomers can be separated by purification techniques known in art (e.g. column chromatography, crystallization). Bromination of intermediate XII provides the desired intermediate compounds IV, wherein R¹ is O and R²═N. This reaction of intermediate XII with N-bromosuccinimide in solvents such as carbon tetrachloride, chlorobenzene, acetonitrile, using radical initiators such as 1,1′-azobis (cyclohexanecarbonitrile) or azobisisobutyronitrile and is carried out at temperatures of 70 to 100° C. The preferred radical initiator is 1,1′-azobis (cyclohexanecarbonitrile), preferred solvent chlorobenzene and preferred temperature 80° C.

The synthesis of compounds containing different substituents R³ follows similar sequence as in Scheme 3, wherein R³ is bromo. Coupling of intermediate III with intermediate IV, wherein R³ is bromo, provides compounds I as described above. Using standard chemical reactions, such as Suzuki or Stille reaction, the bromo group can be converted e.g. to other R³ substituents such as cycloalkyl, alkoxy and alkenyl. Additional transformations e.g. of ethenyl provide compounds I with other R³ substituents such as ethyl, CN and haloalkyl.

Most of the ketones II are commercially available, however for the ones which are not commercially available, preparation of these can be carried out using methods known in prior art. For examples, scheme 4 depicts various methods known in literature for the synthesis of such ketones.

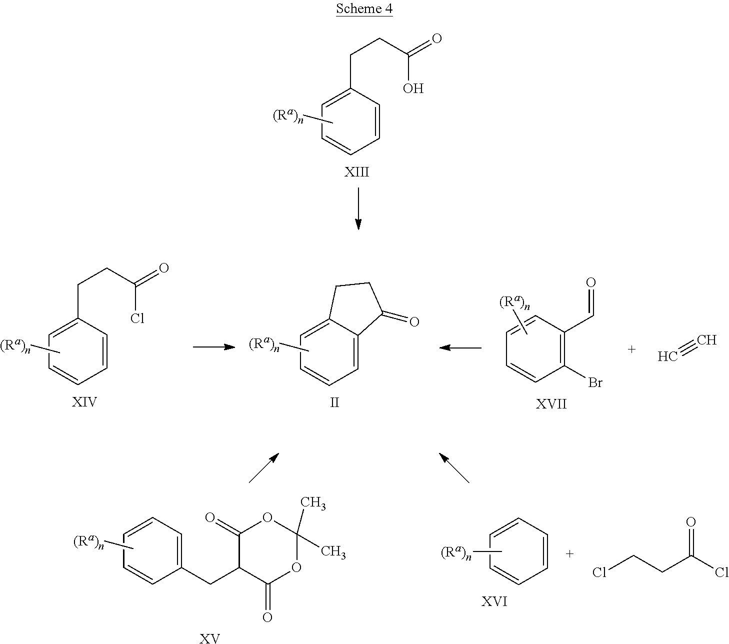

The ketones II can be obtained by the cyclization of 3-arylpropionic acid derivative XIII using catalytic amounts of Bronsted acid such as conc. sulfuric acid (J. Am. Chem. Soc. 1939, 61, 2553-2554) or trifluoromethane sulfonic acid or Lewis acid such as Tb(OTf)₃ (Tetrahedron Lett. 2004, 45, 1741-1745) usually at elevated temperatures. Similarly, ketones II can be accessed via acid chloride XIV by an intramolecular cyclization using an acidic catalyst such as AlCl₃ (Bioorg. Med. Chem. Lett. 2008, 18, 6437-6440) or ZnBr₂ (Can. J. Chem. 2005, 83, 413-419). The synthesis of ketone II can also be achieved by Meldrum acid derivative XV via intramolecular Friedel-Crafts reaction, catalyzed by Sc(OTf)₃, Dy(OTf)₃, or Yb(OTf)₃, using nitromethane as a solvent and a reaction temperature of about 100° C. (J. Org. Chem. 2005, 70, 1316-1327). Alternatively, a Friedel-Crafts reaction of an appropriately substituted benzene derivative XVI with 3-chloropropionyl chloride, using AlCl₃ as a catalyst and nitromethane as solvent at about 25° C. The corresponding acylated product can be cyclized via an intramolecular Friedel-Crafts alkylation in concentrated H₂SO₄ to provide ketone II (J. Med. Chem. 2010, 53, 3675-3684). Ketone II can also be accessed via a palladium catalyzed reaction of appropriately substituted 2-bromo benzaldehyde XVII and an alkyne using DMF as a solvent at temperatures of about 60° C. The resulting indenol can be isomerized to ketone II by heating to about 100° C. (Tetrahedron Lett. 1999, 40, 4089-4092).

The compounds I and the compositions thereof, respectively, are suitable as fungicides effective against a broad spectrum of phytopathogenic fungi, including soil-borne fungi, in particular from the classes of Plasmodiophoromycetes, Peronosporomycetes (syn. Oomycetes), Chytridiomycetes, Zygomycetes, Ascomycetes, Basidiomycetes, and Deuteromycetes (syn. Fungi imperfecti). They can be used in crop protection as foliar fungicides, fungicides for seed dressing, and soil fungicides.

The compounds I and the compositions thereof are preferably useful in the control of phytopathogenic fungi on various cultivated plants, such as cereals, e. g. wheat, rye, barley, triticale, oats, or rice; beet, e. g. sugar beet or fodder beet; fruits, e. g. pomes (apples, pears, etc.), stone fruits (e.g. plums, peaches, almonds, cherries), or soft fruits, also called berries (strawberries, raspberries, blackberries, gooseberries, etc.); leguminous plants, e. g. lentils, peas, alfalfa, or soybeans; oil plants, e. g. oilseed rape, mustard, olives, sunflowers, coconut, cocoa beans, castor oil plants, oil palms, ground nuts, or soybeans; cucurbits, e. g. squashes, cucumber, or melons; fiber plants, e. g. cotton, flax, hemp, or jute; citrus fruits, e. g. oranges, lemons, grapefruits, or mandarins; vegetables, e. g. spinach, lettuce, asparagus, cabbages, carrots, onions, tomatoes, potatoes, cucurbits, or paprika; lauraceous plants, e. g. avocados, cinnamon, or camphor; energy and raw material plants, e. g. corn, soybean, oilseed rape, sugar cane, or oil palm; corn; tobacco; nuts; coffee; tea; bananas; vines (table grapes and grape juice grape vines); hop; turf; sweet leaf (also called Stevia); natural rubber plants; or ornamental and forestry plants, e. g. flowers, shrubs, broad-leaved trees, or evergreens (conifers, eucalypts, etc.); on the plant propagation material, such as seeds; and on the crop material of these plants.

More preferably, compounds I and compositions thereof, respectively are used for controlling fungi on field crops, such as potatoes, sugar beets, tobacco, wheat, rye, barley, oats, rice, corn, cotton, soybeans, oilseed rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant, such as seeds; and vegetative plant materials, such as cuttings and tubers (e. g. potatoes), which can be used for the multiplication of the plant. This includes seeds, roots, fruits, tubers, bulbs, rhizomes, shoots, sprouts and other parts of plants; including seedlings and young plants to be transplanted after germination or after emergence from soil.

Preferably, treatment of plant propagation materials with compounds I and compositions thereof, respectively, is used for controlling fungi on cereals, such as wheat, rye, barley and oats; rice, corn, cotton and soybeans.

According to the invention all of the above cultivated plants are understood to comprise all species, subspecies, variants, varieties and/or hybrids which belong to the respective cultivated plants, including but not limited to winter and spring varieties, in particular in cereals such as wheat and barley, as well as oilseed rape, e.g. winter wheat, spring wheat, winter barley etc.

Corn is also known as Indian corn or maize (Zea mays) which comprises all kinds of corn such as field corn and sweet corn. According to the invention all maize or corn subspecies and/or varieties are comprised, in particular flour corn (Zea mays var. amylacea), popcorn (Zea mays var. everta), dent corn (Zea mays var. indentata), flint corn (Zea mays var. indurata), sweet corn (Zea mays var. saccharata and var. rugosa), waxy corn (Zea mays var. ceratina), amylomaize (high amylose Zea mays varieties), pod corn or wild maize (Zea mays var. tunicata) and striped maize (Zea mays var. japonica).

Most soybean cultivars are classifiable into indeterminate and determinate growth habit, whereas Glycine soja, the wild progenitor of soybean, is indeterminate (PNAS 2010, 107 (19) 8563-8568). The indeterminate growth habit (Maturity Group, MG 00 to MG 4.9) is characterized by a continuation of vegetative growth after flowering begins whereas determinate soybean varieties (MG 5 to MG 8) characteristically have finished most of their vegetative growth when flowering begins. According to the invention all soybean cultivars or varieties are comprised, in particular indeterminate and determinate cultivars or varieties.

The term “cultivated plants” is to be understood as including plants which have been modified by mutagenesis or genetic engineering to provide a new trait to a plant or to modify an already present trait. Mutagenesis includes random mutagenesis using X-rays or mutagenic chemicals, but also targeted mutagenesis to create mutations at a specific locus of a plant genome. Targeted mutagenesis frequently uses oligonucleotides or proteins like CRISPR/Cas, zinc-finger nucleases, TALENs or meganucleases. Genetic engineering usually uses recombinant DNA techniques to create modifications in a plant genome which under natural circumstances cannot readily be obtained by cross breeding, mutagenesis or natural recombination. Typically, one or more genes are integrated into the genome of a plant to add a trait or improve or modify a trait. These integrated genes are also referred to as transgenes, while plant comprising such transgenes are referred to as transgenic plants. The process of plant transformation usually produces several transformation events, which differ in the genomic locus in which a transgene has been integrated. Plants comprising a specific transgene on a specific genomic locus are usually described as comprising a specific “event”, which is referred to by a specific event name. Traits which have been introduced in plants or have been modified include herbicide tolerance, insect resistance, increased yield and tolerance to abiotic conditions, like drought.

The compounds I and compositions thereof, respectively, are particularly suitable for controlling the following causal agents of plant diseases: rusts on soybean and cereals (e.g. Phakopsora pachyrhizi and P. meibomiae on soy; Puccinia tritici and P. striiformis on wheat); molds on specialty crops, soybean, oil seed rape and sunflowers (e.g. Botrytis cinerea on strawberries and vines, Sclerotinia sclerotiorum, S. minor and S. rolfsii on oil seed rape, sunflowers and soybean); Fusarium diseases on cereals (e.g. Fusarium culmorum and F. graminearum on wheat); downy mildews on specialty crops (e.g. Plasmopara viticola on vines, Phytophthora infestans on potatoes); powdery mildews on specialty crops and cereals (e.g. Uncinula necator on vines, Erysiphe spp. on various specialty crops, Blumeria graminis on cereals); and leaf spots on cereals, soybean and corn (e.g. Septoria tritici and S. nodorum on cereals, S. glycines on soybean, Cercospora spp. on corn and soybean).

A further embodiment relates to the use of compound of formula (I) for combating soybean rust on soybean plants and on the plant propagation material, such as seeds, and the crop material of these plants. Soybean rust is cause by two fungal pathogens called Phakopsora pachyrhizi and P. meibomiae.

Consequently, a further embodiment relates to the use of compounds I for combating Phakopsora pachyrhizi and/or P. meibomiae on soybean plants and on the plant propagation material, such as seeds, and the crop material of these plants. A more preferred embodiment the use of compounds I for combating Phakopsora pachyrhizi on soybean plants and on the plant propagation material, such as seeds, and the crop material of these plants.

Accordingly, the present invention relates to the method for combating soybean rust (Phakopsora pachyrhizi and/or P. meibomiae), comprising:

treating the soybean plants or soybean plant propagation material to be protected against attack by Phakopsora pachyrhizi and/or P. meibomiae with an effective amount of at least one compound I, or a composition comprising such compound I.

Treatment against soybean rust can be preventive or curative.

Preferably treatment of soybean plants against soybean rust shall be preventive. Preventive treatment shall be performed when the soybean plants are at risk of infection latest shortly after the first symptoms are visible. According to one embodiment, the first treating of the soybean plants shall take place at the vegetative growth stages V3 to V4 (meaning 4 to 4 fully expanded trifoliate leaves) onwards to the reproductive growth stage R² (full bloom), more preferably place at the vegetative growth stages V6 to V8 (meaning 6 to 8 fully expanded trifoliate leaves) onwards to the reproductive growth stage R³ (beginning bloom). Depending on the disease pressure, two to four and under extreme conditions up to five applications may be necessary at application intervals of 14 to 28 days.

When employed as foliar spray against soybean rust, the amounts of the compounds I applied are, depending on the specific compound used and on the disease pressure, from 5 g to 500 g per ha, preferably from 10 to 200 per ha, more preferably from 15 to 150 g per ha, and in particular from 30 to 125 g per ha.

Furthermore, the present invention relates to the use of compounds of formula I as defined herein for combating phytopathogenic fungi containing an amino acid substitution F129L in the mitochondrial cytochrome b protein conferring resistance to Qo inhibitors.

The mutation F129L in the cytochrome b (cytb, also referred to as cob) gene shall mean any substitution of nucleotides of codon 129 encoding “F” (phenylalanine; e.g. TTT or TTC) that leads to a codon encoding “L” (leucine; e.g. TTA, TTG, TTG, CTT, CTC, CTA or CTG), for example the substitution of the first nucleotide of codon 129 ‘T’ to ‘C’ (TTT to CTT), in the cytochrome b gene resulting in a single amino acid substitution in the position 129 from F (phenylalanine) to L (leucine) (F129L) in the cytochrome b protein (Cytb). In the present invention, the mutation F129L in the cytochrome b gene shall be understood to be a single amino acid substitution in the position 129 from F (phenylalanine) to L (leucine) (F129L) in the cytochrome b protein.

Many other phytopathogenic fungi acquired the F129L mutation in the cytochrome b gene conferring resistance to Qo inhibitors, such as rusts, in particular soybean rust (Phakopsora pachyrhizi and Phakopsora meibromiae) as well as fungi from the genera Alternaria, Pyrenophora and Rhizoctonia. Preferred fungal species are Alternaria solani, Phakopsora pachyrhizi, Phakopsora meibromiae, Pyrenophora teres, Pyrenophora tritici-repentis and Rhizoctonia solani; in particular Phakopsora pachyrhizi.

In one aspect, the present invention relates to the method of protecting plants susceptible to and/or under attack by phytopathogenic fungi containing an amino acid substitution F129L in the mitochondrial cytochrome b protein conferring resistance to Qo inhibitors, which method comprises applying to said plants, treating plant propagation material of said plants with, and/or applying to said phytopathogenic fungi, at least one compound of formula I or a composition comprising at least one compound of formula I.

According to another embodiment, the method for combating phytopathogenic fungi, comprises: a) identifying the phytopathogenic fungi containing an amino acid substitution F129L in the mitochondrial cytochrome b protein conferring resistance to Qo inhibitors, or the materials, plants, the soil or seeds that are at risk of being diseased from phytopathogenic fungi as defined herein, and b) treating said fungi or the materials, plants, the soil or plant propagation material with an effective amount of at least one compound of formula I, or a composition comprising it thereof.

The term “phytopathogenic fungi an amino acid substitution F129L in the mitochondrial cytochrome b protein conferring resistance to Qo inhibitors” is to be understood that at least 10% of the fungal isolates to be controlled contain a such F129L substitution in the mitochondrial cytochrome b protein conferring resistance to Qo inhibitors, preferably at least 30%, more preferably at least 50%, even more preferably at at least 75% of the fungi, most preferably between 90 and 100%; in particular between 95 and 100%.

The compounds I and compositions thereof, respectively, are also suitable for controlling harmful microorganisms in the protection of stored products or harvest, and in the protection of materials.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

The compounds I are employed as such or in form of compositions by treating the fungi, the plants, plant propagation materials, such as seeds; soil, surfaces, materials, or rooms to be protected from fungal attack with a fungicidally effective amount of the active substances. The application can be carried out both before and after the infection of the plants, plant propagation materials, such as seeds; soil, surfaces, materials or rooms by the fungi.

An agrochemical composition comprises a fungicidally effective amount of a compound I. The term “fungicidally effective amount” denotes an amount of the composition or of the compounds I, which is sufficient for controlling harmful fungi on cultivated plants or in the protection of stored products or harvest or of materials and which does not result in a substantial damage to the treated plants, the treated stored products or harvest, or to the treated materials. Such an amount can vary in a broad range and is dependent on various factors, such as the fungal species to be controlled, the treated cultivated plant, stored product, harvest or material, the climatic conditions and the specific compound I used.

Plant propagation materials may be treated with compounds I as such or a composition comprising at least one compound I prophylactically either at or before planting or transplanting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials, such as seeds, e. g. by dusting, coating, or drenching, amounts of active substance of generally from 0.1 to 1000 g, preferably from 1 to 1000 g, more preferably from 1 to 100 g and most preferably from 5 to 100 g, per 100 kg of plant propagation material (preferably seeds) are required.

The user applies the agrochemical composition usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types (see also “Catalogue of pesticide formulation types and international coding system”, Technical Monograph No. 2, 6^{th} Ed. May 2008, CropLife International) are suspensions (e. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel formulations for the treatment of plant propagation materials, such as seeds (e. g. GF). The compositions are prepared in a known manner, such as described by Mollet and Grubemann, Formulation technology, Wiley VCH, Weinheim, 2001; or by Knowles, New developments in crop protection product formulation, Agrow Reports DS243, T&F Informa, London, 2005. The invention also relates to agrochemical compositions comprising an auxiliary and at least one compound I.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers, and binders.

The agrochemical compositions generally comprise between 0.01 and 95%, preferably between 0.1 and 90%, more preferably between 1 and 70%, and in particular between 10 and 60%, by weight of active substances (e.g. at least one compound I). The agrochemical compositions generally comprise between 5 and 99.9%, preferably between 10 and 99.9%, more preferably between 30 and 99%, and in particular between 40 and 90%, by weight of at least one auxiliary. The active substances (e.g. compounds 1) are employed in a purity of from 90% to 100%, preferably from 95-% to 100% (according to NMR spectrum).

Various types of oils, wetters, adjuvants, fertilizers, or micronutrients, and further pesticides (e. g. fungicides, growth regulators, herbicides, insecticides, safeners) may be added to the compounds I or the compositions thereof as premix, or, not until immediately prior to use (tank mix). These agents can be admixed with the compositions according to the invention in a weight ratio of 1:100 to 100:1, preferably 1:10 to 10:1.

Mixing the compounds I or the compositions comprising them in the use form as fungicides with other fungicides results in many cases in an expansion of the fungicidal spectrum of activity or in a prevention of fungicide resistance development. Furthermore, in many cases, synergistic effects are obtained (synergistic mixtures).

The following list of pesticides 1l, in conjunction with which the compounds I can be used, is intended to illustrate the possible combinations but does not limit them:

### A) Respiration Inhibitors

Inhibitors of complex III at Qₒ site: azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxystrobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21), methyl-N-[2-[(1,4-dimethyl-5-phenyl-pyrazol-3-yl)oxylmethyl]phenyl]-N-methoxy-carbamate (A.1.22), metyltetraprole (A.1.25), (Z,2E)-5-[1-(2,4-dichlorophenyl)pyrazol-3-yl]-oxy-2-ethoxyimino-N,3-dimethylpent-3-enamide (A.1.34), (Z,2E)-5-[1-(4-chlorophenyl)pyrazol-3-yl]oxy-2-methoxyimino-N,3-dimethyl-pent-3-enamide (A.1.35), pyriminostrobin (A.1.36), bifujunzhi (A.1.37), 2-(ortho-((2,5-dimethylphenyl-oxymethylen)phenyl)-3-methoxy-acrylic acid methylester (A.1.38);
inhibitors of complex III at Qᵢ site: cyazofamid (A.2.1), amisulbrom (A.2.2), [(6S,7R,8R)-8-benzyl-3-[(3-hydroxy-4-methoxy-pyridine-2-carbonyl)amino]-6-methyl-4,9-dioxo-1,5-dioxonan-7-yl] 2-methylpropanoate (A.2.3), fenpicoxamid (A.2.4), florylpicoxamid (A.2.5), metarylpicoxamid (A.2.6);
inhibitors of complex II: benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.15), penthiopyrad (A.3.16), pydiflumetofen (A.3.17), pyraziflumid (A.3.18), sedaxane (A.3.19), tecloftalam (A.3.20), thifluzamide (A.3.21), inpyrfluxam (A.3.22), pyrapropoyne (A.3.23), fluindapyr (A.3.28), N-[2-[2-chloro-4-(trifluoromethyl)phenoxy]phenyl]-3-(difluoromethyl)-5-fluoro-1-methyl-pyrazole-4-carboxamide (A.3.29), methyl (E)-2-[2-[(5-cyano-2-methyl-phenoxy)methyl]phenyl]-3-methoxy-prop-2-enoate (A.3.30), isoflucypram (A.3.31), 2-(difluoromethyl)-N-(1,1,3-trimethyl-indan-4-yl)pyridine-3-carboxamide (A.3.32), 2-(difluoromethyl)-N-[(3R)-1,1,3-trimethylindan-4-yl]-pyridine-3-carboxamide (A.3.33), 2-(difluoromethyl)-N-(3-ethyl-1,1-dimethyl-indan-4-yl)pyridine-3-carboxamide (A.3.34), 2-(difluoromethyl)-N-[(3R)-3-ethyl-1,1-dimethyl-indan-4-yl]-pyridine-3-carboxamide (A.3.35), 2-(difluoromethyl)-N-(1,1-dimethyl-3-propyl-indan-4-yl)pyridine-3-carboxamide (A.3.36), 2-(difluoromethyl)-N-[(3R)-1,1-dimethyl-3-propyl-indan-4-yl]-pyridine-3-carboxamide (A.3.37), 2-(difluoromethyl)-N-(3-isobutyl-1,1-dimethyl-indan-4-yl)pyridine-3-carboxamide (A.3.38), 2-(difluoromethyl)-N-[(3R)-3-isobutyl-1,1-dimethyl-indan-4-yl]pyridine-3-carboxamide (A.3.39) cyclobutrifluram (A.3.24);
other respiration inhibitors: diflumetorim (A.4.1); nitrophenyl derivates: binapacryl (A.4.2), dinobuton (A.4.3), dinocap (A.4.4), fluazinam (A.4.5), meptyldinocap (A.4.6), ferimzone (A.4.7); organometal compounds: fentin salts, e. g. fentin-acetate (A.4.8), fentin chloride (A.4.9) or fentin hydroxide (A.4.10); ametoctradin (A.4.11); silthiofam (A.4.12);

### B) Sterol Biosynthesis Inhibitors (SBI Fungicides)

C14 demethylase inhibitors: triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 2-(2,4-difluorophenyl)-1,1-difluoro-3-(tetrazol-1-yl)-1-[5-[4-(2,2,2-trifluoroethoxy)phenyl]-2-pyridyl]propan-2-ol (B.1.31), 2-(2,4-difluorophenyl)-1,1-difluoro-3-(tetrazol-1-yl)-1-[5-[4-(trifluoromethoxy)phenyl]-2-pyridyl]propan-2-ol (B.1.32), fluoxytioconazole (B.1.33), ipfentrifluconazole (B.1.37), mefentrifluconazole (B.1.38), (2R)-2-[4-(4-chlorophenoxy)-2-(trifluoromethyl)phenyl]-1-(1,2,4-triazol-1-yl)propan-2-ol, (2S)-2-[4-(4-chlorophenoxy)-2-(trifluoromethyl)phenyl]-1-(1,2,4-triazol-1-yl)propan-2-ol, 2-(chloromethyl)-2-methyl-5-(p-tolylmethyl)-1-(1,2,4-triazol-1-ylmethyl)cyclopentanol (B.1.43); imidazoles: imazalil (B.1.44), pefurazoate (B.1.45), prochloraz (B.1.46), triflumizol (B.1.47); pyrimidines, pyridines, piperazines: fenarimol (B.1.49), pyrifenox (B.1.50), triforine (B.1.51), [3-(4-chloro-2-fluoro-phenyl)-5-(2,4-difluorophenyl)isoxazol-4-yl]-(3-pyridyl)methanol (B.1.52), 4-[[6-[2-(2,4-difluorophenyl)-1,1-difluoro-2-hydroxy-3-(1,2,4-triazol-1-yl)propyl]-3-pyridyl]oxy]benzonitrile (B.1.53), 2-[6-(4-bromophenoxy)-2-(trifluoromethyl)-3-pyridyl]-1-(1,2,4-triazol-1-yl)propan-2-ol (B.1.54), 2-[6-(4-chlorophenoxy)-2-(trifluoromethyl)-3-pyridyl]-1-(1,2,4-triazol-1-yl)propan-2-ol (B.1.55);
Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
Inhibitors of 3-keto reductase: fenhexamid (B.3.1);
Other Sterol biosynthesis inhibitors: chlorphenomizole (B.4.1);

### C) Nucleic Acid Synthesis Inhibitors

phenylamides or acyl amino acid fungicides: benalaxyl (C.1.1), benalaxyl-M (C.1.2), kiralaxyl (C.1.3), metalaxyl (C.1.4), metalaxyl-M (C.1.5), ofurace (C.1.6), oxadixyl (C.1.7);
other nucleic acid synthesis inhibitors: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7), 5-fluoro-2-(4-chlorophenylmethoxy)pyrimidin-4 amine (C.2.8);

### D) Inhibitors of Cell Division and Cytoskeleton

tubulin inhibitors: benomyl (D.1.1), carbendazim (D.1.2), fuberidazole (D1.3), thiabendazole (D.1.4), thiophanate-methyl (D.1.5), pyridachlometyl (D.1.6), N-ethyl-2-[(3-ethynyl-8-methyl6-quinolyl)oxy]butanamide (D.1.8), N-ethyl-2-[(3-ethynyl-8-methyl-6-quinolyl)oxy]-2-methylsulfanyl-acetamide (D.1.9), 2-[(3-ethynyl-8-methyl-6-quinolyl)oxy]-N-(2-fluoroethyl)butanamide (D.1.10), 2-[(3-ethynyl-8-methyl-6-quinolyl)oxy]-N-(2-fluoroethyl)-2-methoxy-acetamide (D.1.11), 2-[(3-ethynyl-8-methyl-6-quinolyl)oxy]-N-propyl-butanamide (D.1.12), 2-[(3-ethynyl-8-methyl-6-quinolyl)oxy]-2-methoxy-N-propyl-acetamide (D.1.13), 2-[(3-ethynyl-8-methyl-6-quinolyl)oxy]-2-methylsulfanyl-N-propyl-acetamide (D.1.14), 2-[(3-ethynyl-8-methyl-6-quinolyl)oxy]-N-(2-fluoroethyl)-2-methylsulfanyl-acetamide (D.1.15), 4-(2-bromo-4-fluorophenyl)-N-(2-chloro-6-fluoro-phenyl)-2,5-dimethyl-pyrazol-3-amine (D.1.16);
other cell division inhibitors: diethofencarb (D.2.1), ethaboxam (D.2.2), pencycuron (D.2.3), fluopicolide (D.2.4), zoxamide (D.2.5), metrafenone (D.2.6), pyriofenone (D.2.7), phenamacril (D.2.8);

### E) Inhibitors of Amino Acid and Protein Synthesis

methionine synthesis inhibitors: cyprodinil (E.1.1), mepanipyrim (E.1.2), pyrimethanil (E.1.3);
protein synthesis inhibitors: blasticidin-S(E.2.1), kasugamycin (E.2.2), kasugamycin hydrochloride-hydrate (E.2.3), mildiomycin (E.2.4), streptomycin (E.2.5), oxytetracyclin (E.2.6);

### F) Signal Transduction Inhibitors

MAP/histidine kinase inhibitors: fluoroimid (F.1.1), iprodione (F.1.2), procymidone (F.1.3), vinclozolin (F.1.4), fludioxonil (F.1.5);
G protein inhibitors: quinoxyfen (F.2.1);

### G) Lipid and Membrane Synthesis Inhibitors

Phospholipid biosynthesis inhibitors: edifenphos (G.1.1), iprobenfos (G.1.2), pyrazophos (G.1.3), isoprothiolane (G.1.4);
lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7), zinc thiazole (G.2.8);
phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7);
compounds affecting cell membrane permeability and fatty acides: propamocarb (G.4.1);
inhibitors of oxysterol binding protein: oxathiapiprolin (G.5.1), fluoxapiprolin (G.5.3), 4-[1-[2-[3-(difluoromethyl)-5-methyl-pyrazol-1-yl]acetyl]-4-piperidyl]-N-tetralin-1-yl-pyridine2-carboxamide (G.5.4), 4-[1-[2-[3,5-bis(difluoromethyl)pyrazol-1-yl]acetyl]-4-piperidyl]-N-tetralin-1-yl-pyridine-2-carboxamide (G.5.5), 4-[1-[2-[3-(difluoromethyl)-5-(trifluoromethyl)pyrazol-1-yl]acetyl]-4-piperidyl]-N-tetralin-1-yl-pyridine-2-carboxamide (G.5.6), 4-[1-[2-[5-cyclopropyl-3-(difluoromethyl)pyrazol-1-yl]acetyl]-4-piperidyl]-N-tetralin-1-yl-pyridine-2-carboxamide (G.5.7), 4-[1-[2-[5-methyl-3-(trifluoromethyl)pyrazol-1-yl]acetyl]-4-piperidyl]-N-tetralin-1-yl-pyridine-2-carboxamide (G.5.8), 4-[1-[2-[5-(difluoromethyl)-3-(trifluoromethyl)pyrazol-1-yl]acetyl]-4-piperidyl]-N-tetralin-1-yl-pyridine-2-carboxamide (G.5.9), 4-[1-[2-[3,5-bis(trifluoromethyl)pyrazol-1-yl]acetyl]-4-piperidyl]-N-tetralin-1-yl-pyridine-2-carboxamide (G.5.10), (4-[1-[2-[5-cyclopropyl-3-(trifluoromethyl)pyrazol-1-yl]acetyl]-4-piperidyl]-N-tetralin-1-yl-pyridine-2-carboxamide (G.5.11);
   H) Inhibitors with Multi Site Action
inorganic active substances: Bordeaux mixture (H.1.1), copper (H.1.2), copper acetate (H.1.3), copper hydroxide (H.1.4), copper oxychloride (H.1.5), basic copper sulfate (H.1.6), sulfur (H.1.7);
thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
organochlorine compounds: anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11);
guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);

### I) Cell Wall Synthesis Inhibitors

inhibitors of glucan synthesis: validamycin (1.1.1), polyoxin B (1.1.2);
melanin synthesis inhibitors: pyroquilon (1.2.1), tricyclazole (1.2.2), carpropamid (1.2.3), dicyclomet (1.2.4), fenoxanil (1.2.5);

### J) Plant Defence Inducers

acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), calcium phosphonate (J.1.11), potassium phosphonate (J.1.12), potassium or sodium bicarbonate (J.1.9), 4-cyclopropyl-N-(2,4-dimethoxyphenyl)thiadiazole-5-carboxamide (J.1.10);

### K) Unknown Mode of Action

bronopol (K.1.1), chinomethionat (K.1.2), cyflufenamid (K.1.3), cymoxanil (K.1.4), dazomet (K.1.5), debacarb (K.1.6), diclocymet (K.1.7), diclomezine (K.1.8), difenzoquat (K.1.9), difenzoquat-methylsulfate (K.1.10), diphenylamin (K.1.11), fenitropan (K.1.12), fenpyrazamine (K.1.13), flumetover (K.1.14), flumetylsulforim (K.1.60), flusulfamide (K.1.15), flutianil (K.1.16), harpin (K.1.17), methasulfocarb (K.1.18), nitrapyrin (K.1.19), nitrothal-isopropyl (K.1.20), tolprocarb (K.1.21), oxin-copper (K.1.22), proquinazid (K.1.23), seboctylamine (K.1.61), tebufloquin (K.1.24), tecloftalam (K.1.25), triazoxide (K.1.26), N′-(4-(4-chloro-3-trifluoromethyl-phenoxy)-2,5-dimethyl-phenyl)-N-ethyl-N-methyl formamidine (K.1.27), N-(4-(4-fluoro-3-trifluoromethyl-phenoxy)-2,5-dimethyl-phenyl)-N-ethyl-N-methyl formamidine (K.1.28), N′-[4-[[3-[(4-chlorophenyl)methyl]-1,2,4-thiadiazol-5-yl]oxy]-2,5-dimethyl-phenyl]-N-ethyl-N-methyl-formamidine (K.1.29), N′-(5-bromo-6-indan-2-yloxy-2-methyl-3-pyridyl)-N-ethyl-N-methyl-formamidine (K.1.30), N′-[5-bromo-6-[1-(3,5-difluorophenyl)ethoxy]-2-methyl3-pyridyl]-N-ethyl-N-methyl-formamidine (K.1.31), N′-[5-bromo-6-(4-isopropylcyclohexoxy)-2-methyl-3-pyridyl]-N-ethyl-N-methyl-formamidine (K.1.32), N′-[5-bromo-2-methyl-6-(1-phenylethoxy)-3-pyridyl]-N-ethyl-N-methyl-formamidine (K.1.33), N′-(2-methyl-5-trifluoromethyl-4-(3-trimethylsilanyl-propoxy)-phenyl)-N-ethyl-N-methyl formamidine (K.1.34), N′-(5-difluoromethyl-2-methyl-4-(3-trimethylsilanyl-propoxy)-phenyl)-N-ethyl-N-methyl formamidine (K.1.35), 2-(4-chloro-phenyl)-N-[4-(3,4-dimethoxy-phenyl)-isoxazol-5-yl]-2-prop-2-ynyloxy-acetamide (K.1.36), 3-[5-(4-chloro-phenyl)-2,3-dimethyl-isoxazolidin-3-yl]-pyridine (pyrisoxazole) (K.1.37), 3-[5-(4-methylphenyl)-2,3-dimethyl-isoxazolidin-3-yl]-pyridine (K.1.38), 5-chloro-1-(4,6-dimethoxy-pyrimidin-2-yl)-2-methyl-1H-benzoimidazole (K.1.39), ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), but-3-ynyl N-[6-[[(Z)-[(1-methyltetrazol-5-yl)-phenyl-methylene]amino]oxymethyl]-2-pyridyl]carbamate (K.1.43), ipflufenoquin (K.1.44), quinofumelin (K.1.47), benziothiazolinone (K.1.48), bromothalonil (K.1.49), 2-(6-benzyl-2-pyridyl)quinazoline (K.1.50), 2-[6-(3-fluoro-4-methoxy-phenyl)-5-methyl-2-pyridyl]quinazoline (K.1.51), dichlobentiazox (K.1.52), N′-(2,5-dimethyl-4-phenoxy-phenyl)-N-ethyl-N-methyl-formamidine (K.1.53), aminopyrifen (K.1.54), fluopimomide (K.1.55), N′-[5-bromo-2-methyl-6-(1-methyl-2-propoxy-ethoxy)-3-pyridyl]-N-ethyl-N-methyl-formamidine (K.1.56), N′-[4-(4,5-dichlorothiazol-2-yl)oxy-2,5-dimethylphenyl]-N-ethyl-N-methyl-formamidine (K.1.57), flufenoxadiazam (K.1.58), N-methyl-4-[5-(trifluoromethyl)-1,2,4-oxadiazol-3-yl]benzenecarbothioamide (K.1.59), N-methoxy-N-[[4-[5-(trifluoromethyl)-1,2,4-oxadiazol-3-yl]phenyl]methyl]cyclopropanecarboxamide (K.1.60; WO2018/177894, WO 2020/212513), N-((4-[5-(trifluoromethyl)-1,2,4-oxadiazol-3-yl]phenyl)methyl)propanamide (K.1.62), 3,3,3-trifluoro-N-[[3-fluoro-4-[5-(trifluoromethyl)-1,2,4-oxadiazol-3-yl]phenyl]methyl]propanamide (K.1.63), 3,3,3-trifluoro-N-[[2-fluoro-4-[5-(trifluoromethyl)-1,2,4-oxadiazol-3-yl]phenyl]methyl]propanamide (K.1.64), N-[2,3-difluoro-4-[5-(trifluoromethyl)-1,2,4-oxadiazol-3-yl]benzyl]butanamide (K.1.65), N-[[2,3-difluoro-4-[5-(trifluoromethyl)-1,2,4-oxadiazol-3-yl]phenyl]methyl]-3,3,3-trifluoro-propanamide (K.1.66), 1-methoxy-1-methyl-3-[[4-[5-(trifluoromethyl)-1,2,4-oxadiazol-3-yl]phenyl]methyl]-urea (K.1.67), 1,1-diethyl-3-[[4-[5-[trifluoromethyl]-1,2,4-oxadiazol-3-yl]phenyl]methyl]urea (K.1.68), N,2-dimethoxy-N-[[4-[5-(trifluoromethyl)-1,2,4-oxadiazol-3-yl]phenyl]methyl]propanamide (K.1.69), N-ethyl-2-methyl-N-[[4-[5-(trifluoromethyl)-1,2,4-oxadiazol-3-yl]phenyl]methyl]propanamide (K.1.70), 1-methoxy-3-methyl-1-[[4-[5-(trifluoromethyl)-1,2,4-oxadiazol-3-yl]-phenyl]methyl]urea (K.1.71), 1-[[4-[5-(trifluoromethyl)-1,2,4-oxadiazol-3-yl]phenyl]methyl]pyrrolidin-2-one (K.1.72), 1-[[4-[5-(trifluoromethyl)-1,2,4-oxadiazol-3-yl]phenyl]methyl]piperidin-2-one (K.1.73), 4-[[4-[5-(trifluoromethyl)-1,2,4-oxadiazol-3-yl]phenyl]methyl]morpholin-3-one (K.1.74), 4,4-dimethyl-2-[[4-[5-(trifluoromethyl)-1,2,4-oxadiazol-3-yl]phenyl]methyl]isoxazolidin-3-one (K.1.75), 2-[[4-[5-(trifluoromethyl)-1,2,4-oxadiazol-3-yl]phenyl]methyl]isoxazolidin-3-one (K.1.76), 5,5-dimethyl-2-[[4-[5-(trifluoromethyl)-1,2,4-oxadiazol-3-yl]phenyl]methyl]-isoxazolidin-3-one (K.1.77), 3,3-dimethyl-1-[[4-[5-(trifluoromethyl)-1,2,4-oxadiazol-3-yl]phenyl]methyl]piperidin-2-one (K.1.78), 2-[[4-[5-(trifluoromethyl)-1,2,4-oxadiazol-3-yl]phenyl]methyl]oxazinan-3-one (K.1.79), 1-[[3-fluoro-4-[5-(trifluoromethyl)-1,2,4-oxadiazol-3-yl]phenyl]-methyl]azepan-2-one (K.1.80), 4,4-dimethyl-1-[[4-[5-(trifluoromethyl)-1,2,4-oxadiazol-3-yl]-phenyl]methyl]pyrrolidin-2-one (K.1.81), 5-methyl-1-[[4-[5-(trifluoromethyl)-1,2,4-oxadiazol-3-yl]phenyl]methyl]pyrrolidin-2-one (K.1.82), ethyl 1-[[4-[5-(trifluoromethyl)-1,2,4-oxadiazol-3-yl]phenyl]methyl]pyrazole-4-carboxylate (K.1.83), N-methyl-1-[[4-[5-(trifluoromethyl)-1,2,4-oxadiazol-3-yl]phenyl]methyl]pyrazole-4-carboxamide (K.1.84), N,N-dimethyl-1-[4-[5-(trifluoromethyl)-1,2,4-oxadiazol-3-yl]benzyl]-1H-1,2,4-triazol-3-amine (K.1.85), N-methoxy-N-methyl-1-[[4-[5-(trifluoromethyl)-1,2,4-oxadiazol-3-yl]phenyl]methyl]pyrazole-4-carboxamide (K.1.86), propyl-1-[[4-[5-(trifluoromethyl)-1,2,4-oxadiazol-3-yl]phenyl]methyl]-pyrazole-4-carboxamide (K.1.87), N-methoxy-1-[[4-[5-(trifluoromethyl)-1,2,4-oxadiazol-3-yl]phenyl]methyl]pyrazole-4-carboxamide (K.1.88), N-allyl-N-[[4-[5-(trifluoromethyl)-1,2,4-oxadiazol-3-yl]phenyl]methyl]propanamide (K.1.89), 3-ethyl-1-methoxy-1-[[4-[5-(trifluoromethyl)-1,2,4-oxadiazol-3-yl]phenyl]methyl]urea (K.1.90), 1,3-dimethoxy-1-[[4-[5-(trifluoromethyl)-1,2,4-oxadiazol-3-yl]phenyl]methyl]urea (K.1.91), N-allyl-N-[[4-[5-(trifluoromethyl)-1,2,4-oxadiazol-3-yl]phenyl]methyl]acetamide (K.1.92), N-[4-[5-(trifluoromethyl)-1,2,4-oxadiazol-3-yl]benzyl]cyclopropanecarboxamide (K.1.93), 1-methyl-3-[[4-[5-(trifluoromethyl)-1,2,4-oxadiazol-3-yl]phenyl]methyl]urea (K.1.94), N′-[2-chloro-4-(2-fluorophenoxy)-5-methyl-phenyl]-N-ethyl-N-methyl-formamidine (K.1.95).

In the binary mixtures the weight ratio of the component 1) and the component 2) generally depends from the properties of the components used, usually it is in the range of from 1:10,000 to 10,000:1, often from 1:100 to 100:1, regularly from 1:50 to 50:1, preferably from 1:20 to 20:1, more preferably from 1:10 to 10:1, even more preferably from 1:4 to 4:1 and in particular from 1:2 to 2:1. According to further embodiments, the weight ratio of the component 1) and the component 2) usually is in the range of from 1000:1 to 1:1, often from 100:1 to 1:1, regularly from 50:1 to 1:1, preferably from 20:1 to 1:1, more preferably from 10:1 to 1:1, even more preferably from 4:1 to 1:1 and in particular from 2:1 to 1:1. According to further embodiments, the weight ratio of the component 1) and the component 2) usually is in the range of from 20,000:1 to 1:10, often from 10,000:1 to 1:1, regularly from 5,000:1 to 5:1, preferably from 5,000:1 to 10:1, more preferably from 2,000:1 to 30:1, even more preferably from 2,000:1 to 100:1 and in particular from 1,000:1 to 100:1. According to further embodiments, the weight ratio of the component 1) and the component 2) usually is in the range of from 1:1 to 1:1000, often from 1:1 to 1:100, regularly from 1:1 to 1:50, preferably from 1:1 to 1:20, more preferably from 1:1 to 1:10, even more preferably from 1:1 to 1:4 and in particular from 1:1 to 1:2. According to further embodiments, the weight ratio of the component 1) and the component 2) usually is in the range of from 10:1 to 1:20,000, often from 1:1 to 1:10,000, regularly from 1:5 to 1:5,000, preferably from 1:10 to 1:5,000, more preferably from 1:30 to 1:2,000, even more preferably from 1:100 to 1:2,000 to and in particular from 1:100 to 1:1,000.

In the ternary mixtures, i.e. compositions comprising the component 1) and component 2) and a compound III (component 3), the weight ratio of component 1) and component 2) depends from the properties of the active substances used, usually it is in the range of from 1:100 to 100:1, regularly from 1:50 to 50:1, preferably from 1:20 to 20:1, more preferably from 1:10 to 10:1 and in particular from 1:4 to 4:1, and the weight ratio of component 1) and component 3) usually it is in the range of from 1:100 to 100:1, regularly from 1:50 to 50:1, preferably from 1:20 to 20:1, more preferably from 1:10 to 10:1 and in particular from 1:4 to 4:1. Any further active components are, if desired, added in a ratio of from 20:1 to 1:20 to the component 1). These ratios are also suitable for mixtures applied by seed treatment.

Preference is given to mixtures comprising as component 2) at least one active substance selected from inhibitors of complex III at Qₒ site in group A), more preferably selected from compounds (A.1.1), (A.1.4), (A.1.8), (A.1.9), (A.1.10), (A.1.12), (A.1.13), (A.1.14), (A.1.17), (A.1.21), (A.1.25), (A.1.34) and (A.1.35); particularly selected from (A.1.1), (A.1.4), (A.1.8), (A.1.9), (A.1.13), (A.1.14), (A.1.17), (A.1.25), (A.1.34) and (A.1.35).

Preference is also given to mixtures comprising as component 2) at least one active substance selected from inhibitors of complex III at Qᵢ site in group A), more preferably selected from compounds (A.2.1), (A.2.3), (A.2.4) and (A.2.6); particularly selected from (A.2.3), (A.2.4) and (A.2.6).

Preference is also given to mixtures comprising as component 2) at least one active substance selected from inhibitors of complex II in group A), more preferably selected from compounds (A.3.2), (A.3.3), (A.3.4), (A.3.7), (A.3.9), (A.3.11), (A.3.12), (A.3.15), (A.3.16), (A.3.17), (A.3.18), (A.3.19), (A.3.20), (A.3.21), (A.3.22), (A.3.23), (A.3.24), (A.3.28), (A.3.31), (A.3.32), (A.3.33), (A.3.34), (A.3.35), (A.3.36), (A.3.37), (A.3.38) and (A.3.39); particularly selected from (A.3.2), (A.3.3), (A.3.4), (A.3.7), (A.3.9), (A.3.12), (A.3.15), (A.3.17), (A.3.19), (A.3.22), (A.3.23), (A.3.24), (A.3.31), (A.3.32), (A.3.33), (A.3.34), (A.3.35), (A.3.36), (A.3.37), (A.3.38) and (A.3.39).

Preference is also given to mixtures comprising as component 2) at least one active substance selected from other respiration inhibitors in group A), more preferably selected from compounds (A.4.5) and (A.4.11); in particular (A.4.11).

Preference is also given to mixtures comprising as component 2) at least one active substance selected from C14 demethylase inhibitors in group B), more preferably selected from compounds (B.1.4), (B.1.5), (B.1.8), (B.1.10), (B.1.11), (B.1.12), (B.1.13), (B.1.17), (B.1.18), (B.1.21), (B.1.22), (B.1.23), (B.1.25), (B.1.26), (B.1.29), (B.1.33), (B.1.34), (B.1.37), (B.1.38), (B.1.43), (B.1.46), (B.1.53), (B.1.54) and (B.1.55); particularly selected from (B.1.5), (B.1.8), (B.1.10), (B.1.17), (B.1.22), (B.1.23), (B.1.25), (B.1.33), (B.1.34), (B.1.37), (B.1.38), (B.1.43) and (B.1.46).

Preference is also given to mixtures comprising as component 2) at least one active substance selected from Delta14-reductase inhibitors in group B), more preferably selected from compounds (B.2.4), (B.2.5), (B.2.6) and (B.2.8); in particular (B.2.4).

Preference is also given to mixtures comprising as component 2) at least one active substance selected from phenylamides and acyl amino acid fungicides in group C), more preferably selected from compounds (C.1.1), (C.1.2), (C.1.4) and (C.1.5); particularly selected from (C.1.1) and (C.1.4).

Preference is also given to mixtures comprising as component 2) at least one active substance selected from other nucleic acid synthesis inhibitors in group C), more preferably selected from compounds (C.2.6), (C.2.7) and (C.2.8).

Preference is also given to mixtures comprising as component 2) at least one active substance selected from group D), more preferably selected from compounds (D.1.1), (D.1.2), (D.1.5), (D.2.4) and (D.2.6); particularly selected from (D.1.2), (D.1.5) and (D.2.6).

Preference is also given to mixtures comprising as component 2) at least one active substance selected from group E), more preferably selected from compounds (E.1.1), (E.1.3), (E.2.2) and (E.2.3); in particular (E.1.3).

Preference is also given to mixtures comprising as component 2) at least one active substance selected from group F), more preferably selected from compounds (F.1.2), (F.1.4) and (F.1.5).

Preference is also given to mixtures comprising as component 2) at least one active substance selected from group G), more preferably selected from compounds (G.3.1), (G.3.3), (G.3.6), (G.5.1), (G.5.3), (G.5.4), (G.5.5), G.5.6), G.5.7), (G.5.8), (G.5.9), (G.5.10) and (G.5.11); particularly selected from (G.3.1), (G.5.1) and (G.5.3).

Preference is also given to mixtures comprising as component 2) at least one active substance selected from group H), more preferably selected from compounds (H.2.2), (H.2.3), (H.2.5), (H.2.7), (H.2.8), (H.3.2), (H.3.4), (H.3.5), (H.4.9) and (H.4.10); particularly selected from (H.2.2), (H.2.5), (H.3.2), (H.4.9) and (H.4.10).

Preference is also given to mixtures comprising as component 2) at least one active substance selected from group 1), more preferably selected from compounds (1.2.2) and (1.2.5).

Preference is also given to mixtures comprising as component 2) at least one active substance selected from group J), more preferably selected from compounds (J.1.2), (J.1.5), (J.1.8), (J.1.11) and (J.1.12); in particular (J.1.5).

Preference is also given to mixtures comprising as component 2) at least one active substance selected from group K), more preferably selected from compounds (K.1.41), (K.1.42), (K.1.44), (K.1.47), (K.1.57), (K.1.58) and (K.1.59); particularly selected from (K.1.41), (K.1.44), (K.1.47), (K.1.57), (K.1.58) and (K.1.59).

The compositions comprising mixtures of active ingredients can be prepared by usual means, e. g. by the means given for the compositions of compounds 1.

### EXAMPLES

### Synthetic Process

### Example 1: Methyl (2E)-2-[2-[[(E)-indan-1-ylideneamino]oxymethyl]-3-methyl-phenyl]-2-methoxyimino-acetate

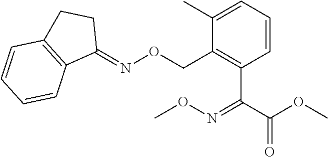 [SMILES: CO/N=C(/C(=O)OC)c1cccc(C)c1CO/N=C1\CCc2ccccc21]

Step 1: To a solution of indan-1-one (7 g, 1 eq.) in 70 mL methanol under nitrogen at about 25° C., pyridine (8.37 g, 2 eq.) and hydroxylamine hydrochloride (7.30 g, 2 eq.) were added and the reaction mixture was heated at 65° C. for 2 h. After TLC indicated completion of the reaction, the reaction mixture was cooled to about 25° C. and concentrated. To the residue 50 mL water was added and extracted with ethyl acetate (2×25 mL). The combined organic phase was dried with sodium sulfate and concentrated to obtain crude product which was purified by flash column chromatography using 10-15% ethyl acetate in heptane as eluent to afford the pure indan-1-one oxime (6 g, 77% yield) as white solid.

¹H NMR (500 MHz, DMSO-d6) δ 10.84 (s, 1H), 7.56 (d, J=7.6 Hz, 1H), 7.39-7.30 (m, 2H), 7.29-7.22 (m, 1H), 3.02-2.96 (m, 2H), 2.82-2.75 (m, 2H).

Step 2: To a stirred solution of indan-1-one oxime (300 mg, 1 eq) in DMF (7 mL), cesium carbonate (1.32 g, 2 eq) and methyl (2E)-2-[2-(bromomethyl)-3-methyl-phenyl]-2-methoxyiminoacetate (670 mg, 1.1 eq) were added at 25° C. The reaction mixture was stirred for 3 h. After TLC showed completion of the reaction, the reaction mixture was quenched with water (10 mL) and extracted with ethyl acetate (2×15 mL). The combined organic phase was washed using brine and dried over sodium sulfate. The solvent was removed to obtain crude product, which was purified by flash column chromatography using 7-9% ethyl acetate in heptane as mobile phase to obtain the title compound (485 mg).

¹H NMR (500 MHz, DMSO-d6) δ 7.49 (d, J=7.7 Hz, 1H), 7.40-7.33 (m, 2H), 7.28 (td, J=7.9, 7.2, 4.4 Hz, 3H), 7.02 (dd, J=6.6, 2.5 Hz, 1H), 4.97 (s, 2H), 3.91 (s, 3H), 3.72 (s, 3H), 3.01-2.95 (m, 2H), 2.74-2.68 (m, 2H), 2.44 (s, 3H).

### Example 2: (2E)-2-[2-[[(E)-indan-1-ylideneamino]oxymethyl]-3-methyl-phenyl]-2-methoxyimino-N-methyl-acetamide

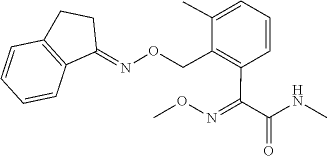 [SMILES: CNC(=O)/C(=N/OC)c1cccc(C)c1CO/N=C1\CCc2ccccc21]

To a stirred solution of methyl (2E)-2-[2-[[(E)-indan-1-ylideneamino]oxymethyl]-3-methylphenyl]-2-methoxyimino-acetate (350 mg, 1 eq) in THF (5 mL), methyl amine (3 mL, 40% in MeOH) was added at 25° C. and the reaction mixture was stirred for 12 h. After TLC showed completion of the reaction, solvents were evaporated. The residue was triturated in n-pentane to provide the title compound as a white solid (290 mg, 83% yield).

¹H NMR (500 MHz, DMSO-d6) δ 8.21 (q, J=4.6 Hz, 1H), 7.53 (d, J=7.7 Hz, 1H), 7.37 (d, J=4.2 Hz, 2H), 7.31-7.23 (m, 3H), 6.95 (dd, J=7.0, 2.1 Hz, 1H), 4.97 (s, 2H), 3.87 (s, 3H), 3.04-2.89 (m, 2H), 2.71 (dd, J=10.8, 5.6 Hz, 5H), 2.44 (s, 3H).

### Example 3: Methyl (2E)-2-methoxyimino-2-[3-methyl-2-[[(E)-tetralin-1-ylideneamino]oxymethyl]-phenyl]acetate

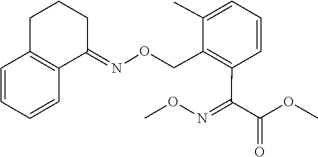 [SMILES: CO/N=C(/C(=O)OC)c1cccc(C)c1CO/N=C1\CCCc2ccccc21]

To a stirred solution of tetralin-1-one oxime (350 mg, 1 eq) in DMF (7 mL), cesium carbonate (1.41 g, 2 eq) and methyl (2E)-2-[2-(bromomethyl)-3-methyl-phenyl]-2-methoxyimino-acetate (717 mg, 1.1 eq) were added at 25° C. The reaction mixture was stirred for 3 h. After TLC showed completion of the reaction, the reaction mixture was quenched with water (10 mL) and the resulting mixture extracted with ethyl acetate (2×15 mL). The combined organic phase was washed using brine and dried over sodium sulfate. The solvent was removed to obtain crude product, which was purified by combi flash column chromatography using 7-9% ethyl acetate in heptane as mobile phase to obtain the title compound (545 mg, 66% yield).

¹H NMR (500 MHz, DMSO-d6) δ 7.50 (d, J=7.7 Hz, 1H), 7.37 (d, J=4.1 Hz, 2H), 7.28 (td, J=7.8, 7.3, 4.5 Hz, 3H), 7.02 (dd, J=6.4, 2.5 Hz, 1H), 4.98 (s, 2H), 3.91 (s, 3H), 3.72 (s, 3H), 3.33 (s, 2H), 3.01-2.95 (m, 2H), 2.74-2.68 (m, 2H), 2.44 (s, 3H).

### Example 4: (2E)-2-methoxyimino-N-methyl-2-[3-methyl-2-[[(E)-tetralin-1-ylideneamino]oxymethyl]phenyl]acetamide

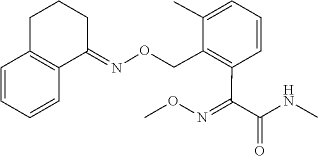 [SMILES: CNC(=O)/C(=N/OC)c1cccc(C)c1CO/N=C1\CCCc2ccccc21]

To a stirred solution of methyl (2E)-2-[2-[[(E)-indan-1-ylideneamino]oxymethyl]-3-methyl-phenyl]-2-methoxyimino-acetate (300 mg, 1 eq) in THF (5 mL), methyl amine (3 mL, 40% in MeOH) was added at 25° C. and the reaction mixture was stirred for 12 h. After TLC showed completion of the reaction, solvents were evaporated. The residue was triturated in n-pentane to provide the title compound as a white solid (240 mg, 80% yield).

¹H NMR (500 MHz, DMSO-d6) δ 8.22 (q, J=4.7 Hz, 1H), 7.78 (d, J=7.8 Hz, 1H), 7.27 (d, J=7.7 Hz, 3H), 7.18 (t, J=7.1 Hz, 2H), 6.98-6.93 (m, 1H), 4.99 (s, 2H), 3.87 (s, 3H), 2.69 (dd, J=13.8, 5.5 Hz, 5H), 2.57 (t, J=6.6 Hz, 2H), 2.44 (s, 3H), 1.71 (p, J=6.2 Hz, 2H).

### Example 15: Methyl (2E)-2-[2-[[(E)-(7-fluorochroman-4-ylidene)amino]oxymethyl]-3-methylphenyl]-2-methoxyimino-acetate

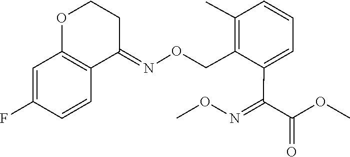 [SMILES: CO/N=C(/C(=O)OC)c1cccc(C)c1CO/N=C1\CCOc2cc(F)ccc21]

Step 1: To a solution of 7-fluorochroman-4-one (410 mg, 1 eq.) in 10 mL methanol under nitrogen, pyridine (0.39 mL, 2 eq.) and hydroxylamine hydrochloride (343 mg, 2 eq.) were added at about 25° C. and the reaction mixture was heated at 70° C. for 2 hours. After TLC indicated completion of the reaction, the reaction mixture was cooled to about 25° C. and concentrated. To the residue, 30 mL ethyl acetate was added. The organic phase was washed with water (3×20 mL) and with brine (1×20 mL) and then dried with sodium sulfate and concentrated to obtain crude 7-fluorochroman-4-one oxime (400 mg, 89.5% yield).

¹H NMR (500 MHz, DMSO-d6) δ 11.24 (s, 1H), 7.81 (dd, J=8.8, 6.8 Hz, 1H), 6.85-6.75 (m, 2H), 4.22 (t, J=6.2 Hz, 2H), 2.83 (t, J=6.2 Hz, 2H).

Step 2: To a suspension of cesium carbonate (1.58 g, 2 eq) in DMF (15 mL), a solution of 7-fluorochroman-4-one oxime (440 mg, 1 eq) in DMF (5 mL) was added. To the resulting mixture, a solution of methyl (2E)-2-[2-(bromomethyl)-3-methyl-phenyl]-2-methoxyimino-acetate (765 mg, 1.05 eq) in DMF (5 mL) was added at about 25° C. and the reaction mixture was stirred for 6 h at about 25° C. After TLC showed completion of the reaction, the reaction mixture was diluted with ethyl acetate (50 mL) and washed with cold water (5×20 mL). The organic phase was dried over sodium sulfate. The solvent was removed to obtain crude product, which was purified by flash column chromatography using 0-30% ethyl acetate in heptane as mobile phase to obtain the title compound (350 mg, 35% yield).

¹H NMR (500 MHz, Chloroform-d) δ 7.81 (dd, J=8.8, 6.7 Hz, 1H), 7.34-7.27 (m, 2H), 7.01 (dd, J=7.3, 1.7 Hz, 1H), 6.64 (td, J=8.5, 2.6 Hz, 1H), 6.56 (dd, J=9.9, 2.6 Hz, 1H), 5.30 (s, OH), 5.09 (s, 2H), 4.17 (t, J=6.2 Hz, 2H), 4.01 (s, 3H), 3.81 (s, 3H), 2.82 (t, J=6.2 Hz, 2H), 2.47 (s, 3H).

### Example 16: (2E)-2-[2-[[(E)-(7-fluorochroman-4-ylidene)amino]oxymethyl]-3-methyl-phenyl]-2-methoxyimino-N-methyl-acetamide

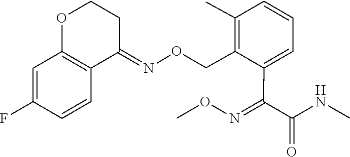 [SMILES: CNC(=O)/C(=N/OC)c1cccc(C)c1CO/N=C1\CCOc2cc(F)ccc21]

To a stirred solution of methyl (2E)-2-[2-[[(E)-(7-fluorochroman-4-ylidene)amino]oxymethyl]-3-methyl-phenyl]-2-methoxyimino-acetate (400 mg, 1 eq) in THF (10 mL), methyl amine (3 mL, 40% in H₂O) was added at 25° C. and the reaction mixture was stirred for 1 h. After TLC showed completion of the reaction, solvents were evaporated, and the residue was diluted with ethyl acetate (25 mL) and washed with water (3×20 mL). The combined organic phase was washed with brine and dried over sodium sulfate. Solvent was removed and the residue was triturated in n-pentane to provide the title compound as a white solid (350 mg, 87% yield).

¹H NMR (500 MHz, Chloroform-d) δ 7.81 (dd, J=8.9, 6.6 Hz, 1H), 7.33-7.22 (m, 3H), 7.01 (dd, J=7.5, 1.4 Hz, 1H), 6.74 (d, J=5.7 Hz, 1H), 6.63 (td, J=8.5, 2.6 Hz, 1H), 6.56 (dd, J=9.9, 2.6 Hz, 1H), 5.07 (d, J=51.3 Hz, 2H), 4.15 (t, J=6.2 Hz, 2H), 3.94 (s, 3H), 2.88 (d, J=5.0 Hz, 3H), 2.81 (t, J=6.2 Hz, 2H), 2.47 (s, 3H).

### Example 25: Methyl (2E)-2-[2-[[(E)-(4-bromoindan-1-ylidene)amino]oxymethyl]-3-methylphenyl]-2-methoxyimino-acetate

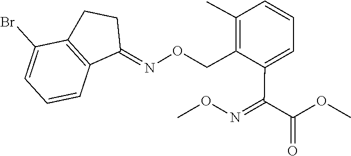 [SMILES: CO/N=C(/C(=O)OC)c1cccc(C)c1CO/N=C1\CCc2c(Br)cccc21]

Step 1: To a solution of 6-bromoindan-1-one (2.5 g, 1 eq.) in 25 mL methanol, under argon at about 25° C. pyridine (1.87 mL, 2 eq.) was added in one portion. Hydroxylamine hydrochloride (1.64 g, 2 eq.) was added and the reaction mixture was heated at 70° C. for 2 h. The reaction mixture was then cooled to about 25° C. and then concentrated by removing solvent. Then 100 mL ethyl acetate was added. The organic phase was washed with water (3×50 mL) and with brine (1×20 mL) and then dried with sodium sulfate and concentrated to obtain crude 4-bromoindan-1-one oxime as pale yellow solid (2.63 g, 98% yield).

¹H NMR (400 MHz, DMSO-d6) δ 11.07 (s, 1H), 7.56 (ddd, J=7.7, 4.2, 0.9 Hz, 2H), 7.23 (t, J=7.7 Hz, 1H), 3.03-2.87 (m, 2H), 2.87-2.73 (m, 2H).

Step 2: To a stirred solution of 4-bromoindan-1-one oxime (2.63 g, 1 eq) in acetonitrile (10 ml), cesium carbonate (7.58 g, 2 eq) was added followed by a solution of (2E)-2-[2-(bromomethyl)-3-methyl-phenyl]-2-methoxyimino-acetate (3.14 g, 0.9 eq) in acetonitrile (15 mL) at about 25° C. The reaction mixture was stirred for 12 h. The reaction mixture was filtered and the filtrate was evaporated. The resulting residue was purified by flash column chromatography to give the title compound (4.37 g, 93% yield).

¹H NMR (400 MHz, DMSO-d6) δ 7.60 (dd, J=7.8, 0.9 Hz, 1H), 7.49 (dd, J=7.7, 0.9 Hz, 1H), 7.34-7.18 (m, 3H), 7.07-6.96 (m, 1H), 4.96 (s, 2H), 3.91 (s, 3H), 3.72 (s, 3H), 2.97-2.84 (m, 2H), 2.84-2.68 (m, 2H), 2.43 (s, 3H).

### Example 25: Methyl (2E)-2-[2-[[(E)-(5-bromoindan-1-ylidene)amino]oxymethyl]-3-methyl-phenyl]-2-methoxyimino-acetate

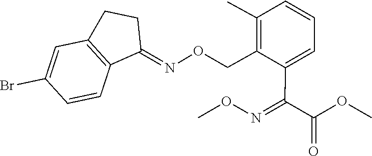 [SMILES: CO/N=C(/C(=O)OC)c1cccc(C)c1CO/N=C1\CCc2cc(Br)ccc21]

Step 1: To a solution of 5-bromoindan-1-one (2.5 g, 1 eq.) in 25 mL methanol, under argon at about 25° C. pyridine (1.90 mL, 2 eq.) was added in one portion. Hydroxylamine hydrochloride (1.64 g, 2 eq.) was added in two portions and heated the reaction mixture at 70° C. for 2 hours. A yellowish-brown reaction mixture was formed. The reaction mixture was then cooled to about 25° C. which resulted in precipitation of a pale yellow solid. The precipitate was filtered and washed with 20 mL pentane and the resulting reddish-brown solution was concentrated. After addition of 70 mL ethyl acetate the organic phase was washed with water (3×20 mL) and with brine (1×20 mL). Then it was dried with sodium sulfate and concentrated to obtain crude 5-bromoindan-1-one oxime (brown solid) as an isomeric mixture (2.58 g, 96% yield).

¹H NMR (400 MHz, DMSO-d6) δ 10.98 (s, 1H), 7.59 (d, J=1.8 Hz, 1H), 7.49-7.41 (m, 2H), 3.03-2.97 (m, 2H), 2.81-2.75 (m, 2H).

Step 2: To a stirred suspension of 5-bromoindan-1-one oxime (1.09 g, 1 eq) in acetonitrile (10 ml), cesium carbonate (3.16 g, 2 eq) was added. Then (2E)-2-[2-(bromomethyl)-3-methylphenyl]-2-methoxyimino-acetate (1.31 g, 0.9 eq) in acetonitrile (5 mL) was added dropwise at about 25° C. The reaction mixture was stirred for 12 h. Then, the reaction mixture was filtered and the filtrate was evaporated. The resulting residue was purified by flash column chromatography to give the title compound (1.29 g, 66% yield).

¹H NMR (400 MHz, DMSO-d6) δ 7.61-7.60 (m, 1H), 7.46 (dd, J=8.3, 1.8 Hz, 1H), 7.40 (d, J=8.3 Hz, 1H), 7.32-7.26 (m, 2H), 7.01 (dd, J=6.5, 2.5 Hz, 1H), 4.97 (s, 2H), 3.90 (s, 3H), 3.71 (s, 3H), 3.00-2.96 (m, 2H), 2.73-2.69 (m, 2H), 2.42 (s, 3H).

### Example 11: Methyl (2E)-2-[2-[[(E)-(6-bromoindan-1-ylidene)amino]oxymethyl]-3-methylphenyl]-2-methoxyimino-acetate

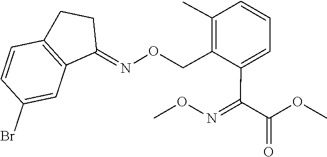 [SMILES: CO/N=C(/C(=O)OC)c1cccc(C)c1CO/N=C1\CCc2ccc(Br)cc21]

Step 1: To a solution of 6-bromoindan-1-one (2.5 g, 1 eq.) in 25 mL methanol, under argon at at about 25° C. pyridine (1.87 mL, 2 eq.) was added in one portion. Hydroxylamine hydrochloride (1.64 g, 2 eq.) was and the reaction mixture was heated at 70° C. for 2 hours. After cooling to about 25° C., the reaction mixture was concentrated by removing solvent. Then 100 mL ethyl acetate was added. The organic phase was washed with water (3×50 mL) and with brine (1×20 mL), dried with sodium sulfate and concentrated to obtain crude 6-bromoindan-1-one oxime as white solid (2.47 g, 92% yield).

¹H NMR (400 MHz, DMSO-d6) δ 11.08 (s, 1H), 7.64 (d, J=1.9 Hz, 1H), 7.50 (dd, J=8.1, 2.0 Hz, 1H), 7.44-7.25 (m, 1H), 3.04-2.87 (m, 2H), 2.88-2.72 (m, 2H).

Step 2: To a stirred suspension of 6-bromoindan-1-one oxime (2.47 g, 1 eq) in acetonitrile (10 ml), cesium carbonate (7.12 g, 2 eq) was added. (2E)-2-[2-(bromomethyl)-3-methyl-phenyl]-2-methoxyimino-acetate (2.95 g, 0.9 eq) in acetonitrile (15 mL) was added dropwise at 25° C. The reaction mixture was stirred for 12 h. The reaction mixture was filtered and the filtrate was evaporated. The resulting residue was purified by flash column chromatography to give methyl the title compound (550 mg, 13% yield).

¹H NMR (400 MHz, DMSO-d6) δ 7.59-7.49 (m, 2H), 7.37-7.23 (m, 3H), 7.06-6.98 (m, 1H), 4.97 (s, 2H), 3.91 (s, 3H), 3.75 (s, 3H), 2.97-2.89 (m, 2H), 2.77-2.68 (m, 2H), 2.43 (s, 3H).

### Example 22: (2E)-2-[2-[[(E)-(6-bromoindan-1-ylidene)amino]oxymethyl]-3-methyl-phenyl]-2-methoxyimino-N-methyl-acetamide

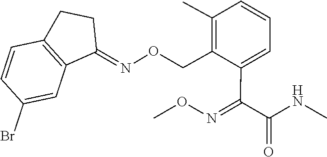 [SMILES: CNC(=O)/C(=N/OC)c1cccc(C)c1CO/N=C1\CCc2ccc(Br)cc21]

To a stirred solution of methyl (2E)-2-[2-[[(E)-(6-bromoindan-1-ylidene)amino]oxymethyl]-3-methyl-phenyl]-2-methoxyimino-acetate (300 mg, 1 eq) in THF (10 mL), methyl amine (0.6 mL, 40% solution in H₂O, 10 eq) was added. Solvents were evaporated under reduced pressure and the residue purified by reverse phase chromatography using acetonitrile and water mixture as a mobile phase to give the title compound (297 mg, 99% yield).

¹H NMR (400 MHz, DMSO-d6) δ 8.21 (d, J=4.9 Hz, 1H), 7.61 (d, J=1.9 Hz, 1H), 7.51 (dd, J=8.1, 2.0 Hz, 1H), 7.35-7.20 (m, 3H), 6.99-6.91 (m, 1H), 4.96 (s, 2H), 3.87 (s, 3H), 2.95-2.87 (m, 2H), 2.73 (t, J=5.1 Hz, 4H), 2.42 (s, 3H).

### Example 36: Methyl (2E)-2-[2-[[(E)-(7-bromoindan-1-ylidene)amino]oxymethyl]-3-methyl-phenyl]-2-methoxyimino-acetate

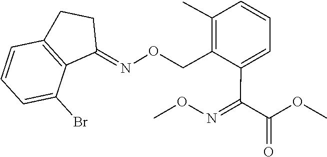 [SMILES: CO/N=C(/C(=O)OC)c1cccc(C)c1CO/N=C1\CCc2cccc(Br)c21]

Step 1: To a solution of 7-bromoindan-1-one (3 g, 1 eq.) in 30 mL methanol, under argon at about 25° C. pyridine (2.29 mL, 2 eq.) was added in one portion. Hydroxylamine hydrochloride (1.98 g, 2 eq.) was added in two portions and the reaction mixture was heated at 70° C. for 3 h.

A yellowish suspension was formed. The reaction mixture was then cooled to about 25° C. and stirred for 48 h. Then, the reaction mixture was concentrated and 70 mL ethyl acetate was added. The organic phase was washed with water (3×20 mL) and with brine (1×20 mL), dried with sodium sulfate and concentrated to obtain crude 7-bromoindan-1-one oxime as pale yellow solid (3.6 g, 89.6% yield).

¹H NMR (400 MHz, DMSO-d6) δ 11.27 (s, 1H), 7.49 (dd, J=7.8, 1.0 Hz, 1H), 7.38 (dq, J=7.5, 1.1 Hz, 1H), 7.23 (t, J=7.7 Hz, 1H), 3.00 (dd, J=8.7, 5.0 Hz, 2H), 2.87-2.82 (m, 2H).

Step 2: To a stirred solution of 7-bromoindan-1-one oxime (1.39 g, 1 eq) in acetonitrile (10 ml), cesium carbonate (4.00 g, 2 eq) was added. (2E)-2-[2-(bromomethyl)-3-methyl-phenyl]-2-methoxyimino-acetate (1.66 g, 0.9 eq) in acetonitrile (10 mL) was added dropwise at 25° C. The reaction mixture was stirred for 12 h. The reaction mixture was filtered and the filtrate was evaporated. The resulting residue was purified by flash column chromatography to give the title compound (385 mg, 16% yield).

¹H NMR (400 MHz, DMSO-d6) δ 7.51-7.48 (m, 1H), 7.38 (d, J=7.6 Hz, 1H), 7.30-7.24 (m, 4H), 7.01 (dd, J=6.2, 2.8 Hz, 1H), 5.02 (s, 2H), 3.92 (s, 3H), 3.72 (s, 3H), 3.00-2.95 (m, 2H), 2.75-2.70 (m, 3H), 2.47 (s, 3H).

### Example 14: Methyl (2E)-2-[2-[[(E)-(3,3-dimethylindan-1-ylidene)amino]oxymethyl]-3-methylphenyl]-2-methoxyimino-acetate

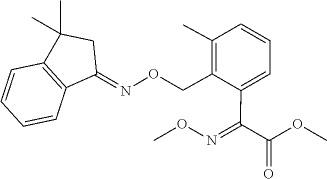 [SMILES: CO/N=C(/C(=O)OC)c1cccc(C)c1CO/N=C1\CC(C)(C)c2ccccc21]

Step 1: To a solution of 3,3-dimethylindan-1-one (2.5 g, 1 eq.) in 30 mL methanol, under argon at about 25° C. pyridine (2.51 mL, 2 eq.) was added in one portion. Hydroxylamine hydrochloride (2.16 g, 2 eq.) was added and the reaction mixture was heated at 70° C. for 2 h. Then, the reaction mixture was cooled to about 25° C. and concentrated by removing solvent. After addition of 100 mL ethyl acetate, the organic phase was washed with water (3×50 mL) and 15 with brine (1×20 mL), dried with sodium sulfate and concentrated to obtain crude 3,3-dimethylindan-1-one oxime as a yellow oil (2.60 g, 95% yield).

¹H NMR (400 MHz, DMSO-d6) δ 10.87 (s, 1H), 7.51 (dt, J=7.6, 1.0 Hz, 1H), 7.43-7.32 (m, 2H), 7.30-7.20 (m, 1H), 2.66 (s, 2H), 1.28 (s, 6H).

Step 2: To a stirred solution of 3,3-dimethylindan-1-one oxime (2.60 g, 1 eq) in acetonitrile (10 ml), cesium carbonate (9.67 g, 2 eq) was added. (2E)-2-[2-(bromomethyl)-3-methyl-phenyl]-2-methoxyimino-acetate (4.00 g, 0.9 eq) in acetonitrile (15 mL) was added dropwise at 25° C. The reaction mixture was stirred for 12 h. The reaction mixture was filtered and the filtrate was evaporated. The resulting residue was purified by flash column chromatography to give the title compound (4.51 g, 85% yield).

### Example 19: (2E)-2-[2-[[(E)-(3,3-dimethylindan-1-ylidene)amino]oxymethyl]-3-methyl-phenyl]-2-methoxyimino-N-methyl-acetamide

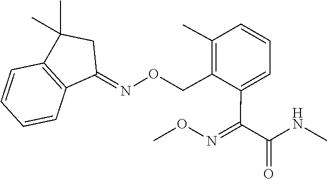 [SMILES: CNC(=O)/C(=N/OC)c1cccc(C)c1CO/N=C1\CC(C)(C)c2ccccc21]

To a stirred solution of methyl 2E)-2-[2-[[(E)-(3,3-dimethylindan-1-ylidene)amino]oxymethyl]-3-methyl-phenyl]-2-methoxyimino-acetate (500 mg, 1 eq) in THF (10 mL), methyl amine (0.9 mL, 40% solution in H₂O, 10 eq) was added and the reaction mixture was stirred for 3 h. Solvents were evaporated under reduced pressure and the residue purified by reverse phase chromatography using acetonitrile and water mixture as a mobile phase to give the title compound (372 mg, 75% yield).

¹H NMR (400 MHz, DMSO-d6) δ 8.19 (t, J=4.8 Hz, 1H), 7.47 (dt, J=7.6, 1.0 Hz, 1H), 7.41-7.36 (m, 2H), 7.30-7.21 (m, 3H), 6.94 (dd, J=6.8, 2.2 Hz, 1H), 4.96 (s, 2H), 3.87 (s, 3H), 2.70 (d, J=4.7 Hz, 3H), 2.58 (s, 2H), 2.43 (s, 3H), 1.24 (s, 6H).

### Example 24: Methyl (2E)-2-[2-[[(E)-[6-fluoro-4-(trifluoromethyl)indan-1-ylidene]amino]oxymethyl]-3-methyl-phenyl]-2-methoxyimino-acetate

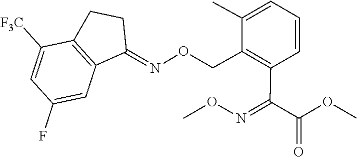 [SMILES: CO/N=C(/C(=O)OC)c1cccc(C)c1CO/N=C1\CCc2c1cc(F)cc2C(F)(F)F]

Step 1: To a solution of 6-fluoro-4-(trifluoromethyl)indan-1-one (2.5 g, 1 eq.) in 25 mL methanol, under argon pyridine (1.84 mL, 2 eq.) was added in one portion. Hydroxylamine hydrochloride (1.59 g, 2 eq.) was added in two portions and the reaction mixture was heated at 70° C. for 2 h.

A yellowish-brown reaction mixture was formed. Then, the reaction mixture was cooled to about 25° C. which resulted in the precipitation of a solid. The precipitate was filtered and the solid was washed with methanol in which it was fully soluble. The red-brown solution was evaporated, and 70 mL ethyl acetate was added to the resulting residue. The organic phase was washed with 15 water (3×20 mL) and with brine (1×20 mL), dried with sodium sulfate and concentrated to obtain crude 6-fluoro-4-(trifluoromethyl)indan-1-one oxime (brown solid) as an isomeric mixture (2.47 g, 92% yield).

¹H NMR (400 MHz, DMSO-d6) δ 11.35 (s, 1H), 7.60 (td, J=8.3, 2.5 Hz, 2H), 3.16-3.06 (m, 2H), 2.93-2.82 (m, 2H).

Step 2: To a stirred solution of 6-fluoro-4-(trifluoromethyl)indan-1-one oxime (2.47 g, 1 eq) in acetonitrile (10 ml), cesium carbonate (6.90 g, 2 eq) was added followed by a solution of (2E)-2-[2-(bromomethyl)-3-methyl-phenyl]-2-methoxyimino-acetate (2.86 g, 0.9 eq) in acetonitrile (15 mL) at about 25° C. The reaction mixture was stirred for 12 h. The reaction mixture was filtered and the filtrate was evaporated. The resulting residue was purified by flash column chromatography to give the title compound (3.81 g, 88% yield).

¹H NMR (400 MHz, DMSO-d6) δ 7.67 (dd, J=9.0, 2.4 Hz, 1H), 7.52-7.48 (m, 1H), 7.31-7.28 (m, 2H), 7.02 (dd, J=6.8, 2.4 Hz, 1H), 5.01 (s, 2H), 3.91 (s, 3H), 3.74 (s, 3H), 3.10 (s, 2H), 2.84-2.79 (m, 2H), 2.43 (s, 3H).

### Example 28: (2E)-2-[2-[[(E)-[6-fluoro-4-(trifluoromethyl)indan-1-ylidene]amino]oxymethyl]-3-methyl-phenyl]-2-methoxyimino-N-methyl-acetamide

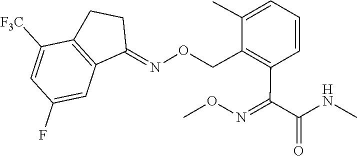 [SMILES: CNC(=O)/C(=N/OC)c1cccc(C)c1CO/N=C1\CCc2c1cc(F)cc2C(F)(F)F]

To a stirred solution of methyl methyl (2E)-2-[2-[[(E)-[6-fluoro-4-(trifluoromethyl)indan-1-ylidene]-amino]oxymethyl]-3-methyl-phenyl]-2-methoxyimino-acetate (800 mg, 1 eq) in THF (7 mL), methyl amine (1.5 mL, 40% solution in H₂O, 10 eq) was added at about 25° C. and the reaction mixture was stirred for 3 h. Solvents were evaporated under reduced pressure and the residue purified by reverse phase chromatography using acetonitrile and water mixture as a mobile phase to give the title compound (513 mg, 64% yield).

1H NMR (400 MHz, DMSO-d6) b 8.24 (d, J=4.8 Hz, 1H), 7.65 (dd, J=9.0, 2.4 Hz, 1H), 7.55 (d, J=8.0 Hz, 1H), 7.26 (d, J=2.1 Hz, 2H), 6.94 (dd, J=7.1, 2.0 Hz, 1H), 5.00 (s, 2H), 3.87 (s, 3H), 3.08 (d, J=7.0 Hz, 2H), 2.85-2.80 (m, 2H), 2.70 (d, J=4.7 Hz, 3H), 2.42 (s, 3H).

The following examples in Table S were synthesized as per general Schemes described above and characterized by LCMS as described in Table L.

TABLE L
LCMS Methods

| | |
|---|---|
| Method details | Device details |

LCMS Method A

| | |
|---|---|
| Column: Agilent Eclipse Plus C18 | LCMS2020 (Shimadzu) |
| (50 mm × 4.6 mm × 3 μm particles) | Ionization source: ESI |
| Mobile Phase: | Mass range: 100-800 amu |
| A: 10 mM Ammonium formate in water. | Polarity: Dual (positive and |
| B: 0.1% Formic acid in acetonitrile. | negative simultaneous scan); |
| Gradient: 10% B to 100% B in 1.5 min. | Mode: Scan |
| Hold 1 min 100% B. 1 min 10% B. | LC System: Nexera High pressure |
| Run time: 3.50 or 3.75 min. | gradient system, Binary pump |
| Flow: 1.2 ml/min; | Detector: PDA |
| Column oven: 30° C./40° C. | Scanning wavelength: 220 nm/max plot |

LCMS Method B

| | |
|---|---|
| Column: Kinetex XB C18 | LCMS2020 (Shimadzu) |
| (50 mm × 2.1 mm × 1.7 μm particles) | Ionization source: ESI |
| Mobile Phase: | Mass range: 100-800 amu |
| A: Water + 0.1% TFA. | Polarity: Dual (positive and |
| B: Acetonitrile | negative simultaneous scan); |
| Gradient: 5% B to 100% B in 1.5 min. | Mode: Scan |
| Flow: 0.8 ml/min to 1.0 ml/min in 1.5 min; | LC System: Nexera High pressure |
| Column oven: 60° C. | gradient system, Binary pump |
| | Detector: PDA |
| | Scanning wavelength: 220 nm/max plot |

LCMS Method C

| | |
|---|---|
| Column: Luna-C18 | LCMS DELIVER-220 (Shimadzu) |
| (30 mm × 2.0 mm × 3 μm particles) | Ionization source: ESI |
| Mobile Phase: | Mass range: 100-1000 amu |
| A: 0.037% Trifluoroacetic acid | Polarity: Positive |
| in water. | Mode: Scan |
| B: 0.018% Trifluoroacetic acid | LC System: Nexera High pressure |
| in HPLC grade acetonitrile. | gradient system, Binary pump |
| Gradient: 5-95% B in 3.00 min .5% B | Detector: DAD |
| in 0.01 min, 5-95% B (0.01-1.60 min), | Scanning wavelength: 220 nm/max plot |
| 95-100% B (1.60-2.50 min), 100-5% | |
| (2.50-2.52 min) with a hold at 5% B | |
| for 0.48 min. | |
| Flow: 0.8 mL/min; Column oven: 40° C. | |

TABLE S

| | | LCMS data | | |
|---|---|---|---|---|
| | Compound | Rₜ | | |
| No. | Structure | [min] | Mass | Method |
| 1 | 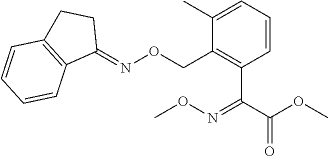 [SMILES: CO/N=C(/C(=O)OC)c1cccc(C)c1CO/N=C1\CCc2ccccc21] | 2.1 | 367 | A |
| 2 | 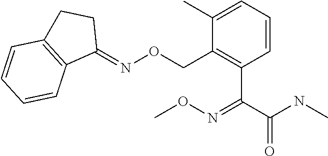 [SMILES: CNC(=O)/C(=N/OC)c1cccc(C)c1CO/N=C1\CCc2ccccc21] | 2.005 | 366 | A |
| 3 | 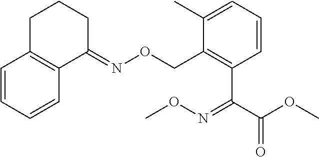 [SMILES: CO/N=C(/C(=O)OC)c1cccc(C)c1CO/N=C1\CCCc2ccccc21] | 2.176 | 381 | A |
| 4 | 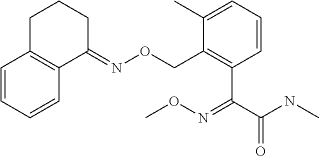 [SMILES: CNC(=O)/C(=N/OC)c1cccc(C)c1CO/N=C1\CCCc2ccccc21] | 2.037 | 380 | A |
| 5 | 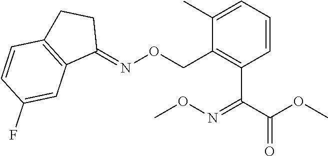 [SMILES: CO/N=C(/C(=O)OC)c1cccc(C)c1CO/N=C1\CCc2ccc(F)cc21] | 2.176 | 385 | A |
| 6 | 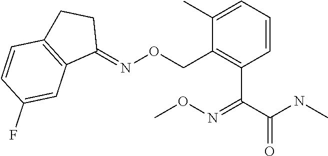 [SMILES: CNC(=O)/C(=N/OC)c1cccc(C)c1CO/N=C1\CCc2ccc(F)cc21] | 2.048 | 384 | A |
| 7 | 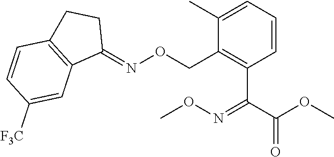 [SMILES: CO/N=C(/C(=O)OC)c1cccc(C)c1CO/N=C1\CCc2ccc(C(F)(F)F)cc21] | 2.176 | 435 | A |
| 8 | 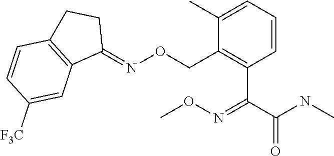 [SMILES: CNC(=O)/C(=N/OC)c1cccc(C)c1CO/N=C1\CCc2ccc(C(F)(F)F)cc21] | 2.05 | 434 | A |
| 9 | 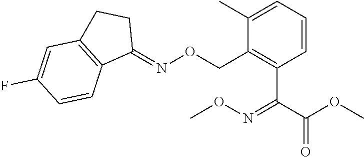 [SMILES: CO/N=C(/C(=O)OC)c1cccc(C)c1CO/N=C1\CCc2cc(F)ccc21] | 2.112 | 385 | A |
| 10 | 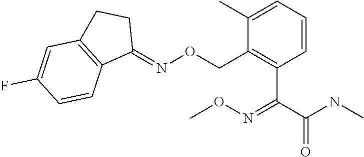 [SMILES: CNC(=O)/C(=N/OC)c1cccc(C)c1CO/N=C1\CCc2cc(F)ccc21] | 1.963 | 384 | A |
| 11 | 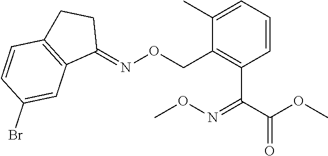 [SMILES: CO/N=C(/C(=O)OC)c1cccc(C)c1CO/N=C1\CCc2ccc(Br)cc21] | 1.371 | 447 | B |
| 12 | 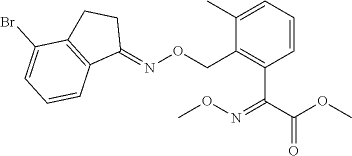 [SMILES: CO/N=C(/C(=O)OC)c1cccc(C)c1CO/N=C1\CCc2c(Br)cccc21] | 1.377 | 446.9 | B |
| 13 | 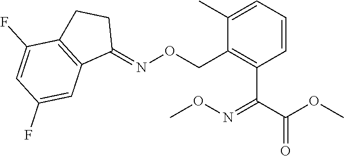 [SMILES: CO/N=C(/C(=O)OC)c1cccc(C)c1CO/N=C1\CCc2c(F)cc(F)cc21] | 1.328 | 403 | B |
| 14 | 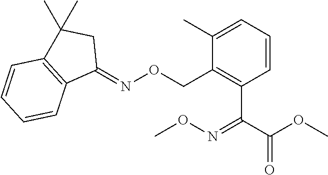 [SMILES: CO/N=C(/C(=O)OC)c1cccc(C)c1CO/N=C1\CC(C)(C)c2ccccc21] | 1.359 | 395 | B |
| 15 | 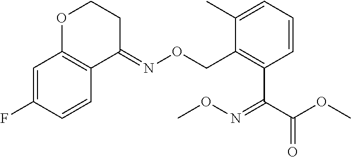 [SMILES: CO/N=C(/C(=O)OC)c1cccc(C)c1CO/N=C1\CCOc2cc(F)ccc21] | 2.187 | 401 | B |
| 16 | 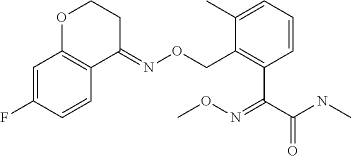 [SMILES: CNC(=O)/C(=N/OC)c1cccc(C)c1CO/N=C1\CCOc2cc(F)ccc21] | 2.048 | 400 | B |
| 17 | 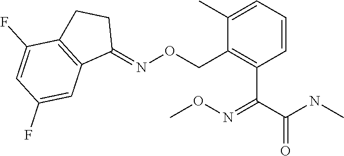 [SMILES: CNC(=O)/C(=N/OC)c1cccc(C)c1CO/N=C1\CCc2c(F)cc(F)cc21] | 1.26 | 402 | B |
| 18 | 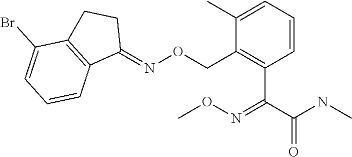 [SMILES: CNC(=O)/C(=N/OC)c1cccc(C)c1CO/N=C1\CCc2c(Br)cccc21] | 1.30 | 444 | B |
| 19 | 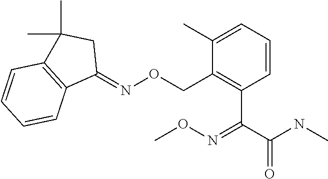 [SMILES: CNC(=O)/C(=N/OC)c1cccc(C)c1CO/N=C1\CC(C)(C)c2ccccc21] | 2.05 | 380 | A |
| 20 | 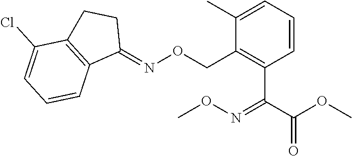 [SMILES: CO/N=C(/C(=O)OC)c1cccc(C)c1CO/N=C1\CCc2c(Cl)cccc21] | 1.39 | 400.8 | B |
| 21 | 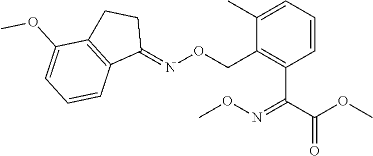 | 1.31 | 396.9 | B |
| 22 | 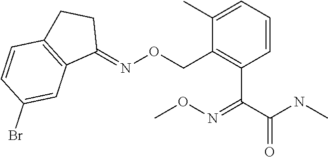 [SMILES: CNC(=O)/C(=N/OC)c1cccc(C)c1CO/N=C1\CCc2ccc(Br)cc21] | 1.32 | 445.7 | B |
| 23 | 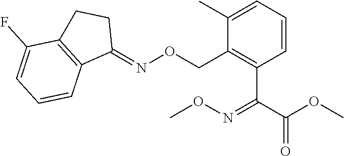 [SMILES: CO/N=C(/C(=O)OC)c1cccc(C)c1CO/N=C1\CCc2c(F)cccc21] | 1.33 | 384.4 | B |
| 24 | 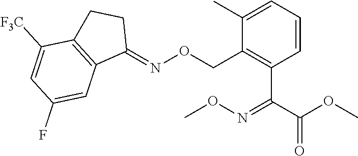 [SMILES: CO/N=C(/C(=O)OC)c1cccc(C)c1CO/N=C1\CCc2c1cc(F)cc2C(F)(F)F] | 1.44 | 452.8 | B |
| 25 | 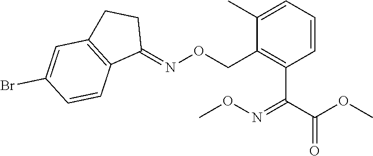 [SMILES: CO/N=C(/C(=O)OC)c1cccc(C)c1CO/N=C1\CCc2cc(Br)ccc21] | 1.40 | 446.6 | B |
| 26 | 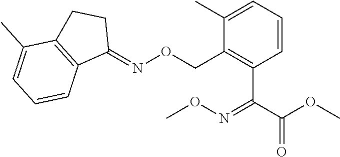 | 1.34 | 381.1 | B |
| 27 | 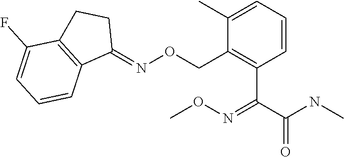 [SMILES: CNC(=O)/C(=N/OC)c1cccc(C)c1CO/N=C1\CCc2c(F)cccc21] | 1.20 | 384 | B |
| 28 | 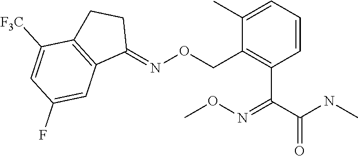 [SMILES: CNC(=O)/C(=N/OC)c1cccc(C)c1CO/N=C1\CCc2c1cc(F)cc2C(F)(F)F] | 1.32 | 452 | B |
| 29 | 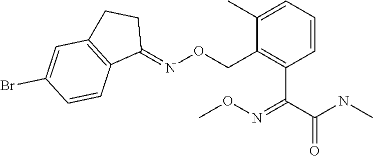 [SMILES: CNC(=O)/C(=N/OC)c1cccc(C)c1CO/N=C1\CCc2cc(Br)ccc21] | 1.28 | 444 | B |
| 30 | 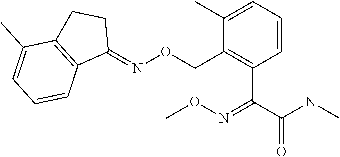 | 1.23 | 380 | B |
| 31 | 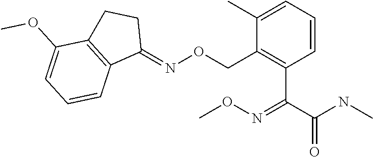 | 1.21 | 396 | B |
| 32 | 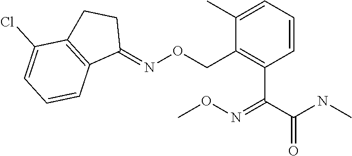 [SMILES: CNC(=O)/C(=N/OC)c1cccc(C)c1CO/N=C1\CCc2c(Cl)cccc21] | 1.3 | 399.8 | B |
| 33 | 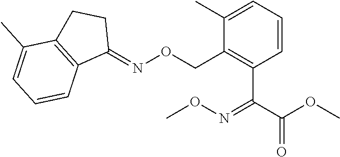 | 1.35 | 380.9 | B |
| 34 | 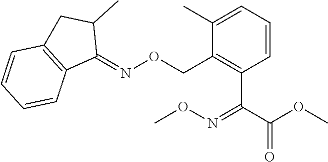 [SMILES: CO/N=C(/C(=O)OC)c1cccc(C)c1CO/N=C1/c2ccccc2CC1C] | 1.36 | 381 | B |
| 35 | 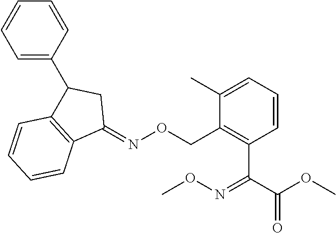 [SMILES: CO/N=C(/C(=O)OC)c1cccc(C)c1CO/N=C1\CC(c2ccccc2)c2ccccc21] | 1.42 | 443 | B |
| 36 | 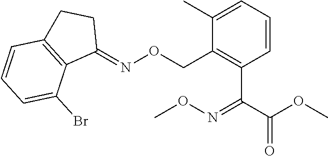 [SMILES: CO/N=C(/C(=O)OC)c1cccc(C)c1CO/N=C1\CCc2cccc(Br)c21] | 1.37 | 446.7 | B |
| 37 | 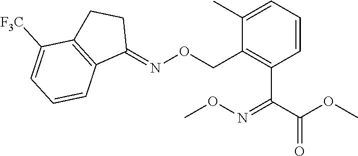 [SMILES: CO/N=C(/C(=O)OC)c1cccc(C)c1CO/N=C1\CCc2c1cccc2C(F)(F)F] | 1.40 | 434.8 | B |
| 38 | 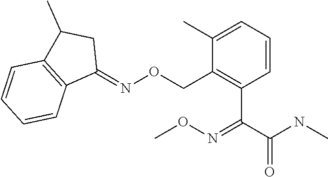 [SMILES: CNC(=O)/C(=N/OC)c1cccc(C)c1CO/N=C1\CC(C)c2ccccc21] | 1.26 | 379.9 | B |
| 39 | 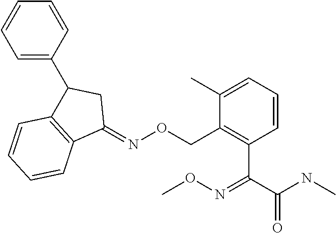 [SMILES: CNC(=O)/C(=N/OC)c1cccc(C)c1CO/N=C1\CC(c2ccccc2)c2ccccc21] | 1.34 | 442.1 | B |
| 40 | 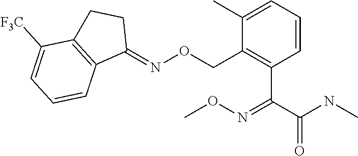 [SMILES: CNC(=O)/C(=N/OC)c1cccc(C)c1CO/N=C1\CCc2c1cccc2C(F)(F)F] | 1.31 | 434.1 | B |
| 41 | 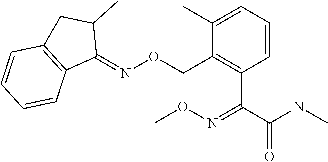 [SMILES: CNC(=O)/C(=N/OC)c1cccc(C)c1CO/N=C1/c2ccccc2CC1C] | 1.26 | 379.9 | B |
| 42 | 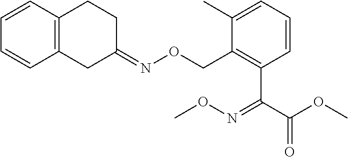 [SMILES: CO/N=C(/C(=O)OC)c1cccc(C)c1CO/N=C1\CCc2ccccc2C1] | 2.09 | 381 | A |
| 43 | 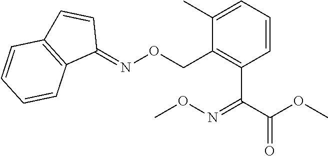 [SMILES: CO/N=C(/C(=O)OC)c1cccc(C)c1CO/N=C1\C=Cc2ccccc21] | 1.3 | 365 | B |
| 44 | 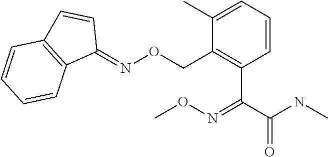 [SMILES: CNC(=O)/C(=N/OC)c1cccc(C)c1CO/N=C1\C=Cc2ccccc21] | 1.21 | 364 | B |
| 45 | 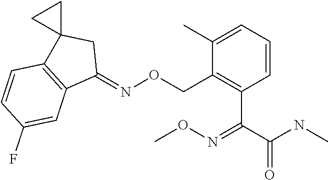 [SMILES: CNC(=O)/C(=N/OC)c1cccc(C)c1CO/N=C1\CC2(CC2)c2ccc(F)cc21] | 1.82 | 410.2 | C |
| 46 | 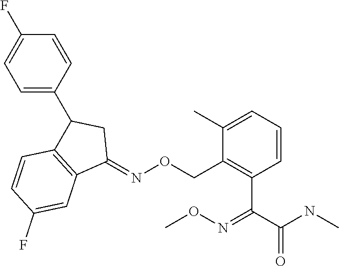 [SMILES: CNC(=O)/C(=N/OC)c1cccc(C)c1CO/N=C1\CC(c2ccc(F)cc2)c2ccc(F)cc21] | 1.91 | 478.2 | C |
| 47 | 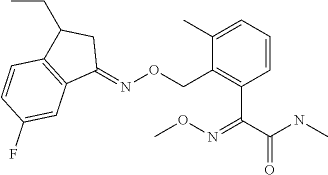 [SMILES: CCC1C/C(=N\OCc2c(C)cccc2/C(=N\OC)C(=O)NC)c2cc(F)ccc21] | 1.72 | 412.2 | C |
| 48 | 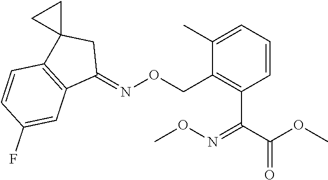 [SMILES: CO/N=C(/C(=O)OC)c1cccc(C)c1CO/N=C1\CC2(CC2)c2ccc(F)cc21] | 1.92 | 411.2 | C |
| 49 | 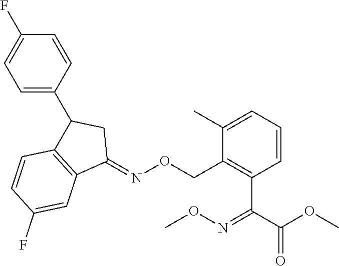 [SMILES: CO/N=C(/C(=O)OC)c1cccc(C)c1CO/N=C1\CC(c2ccc(F)cc2)c2ccc(F)cc21] | 1.99 | 479.2 | C |
| 50 | 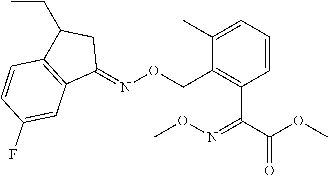 [SMILES: CCC1C/C(=N\OCc2c(C)cccc2/C(=N\OC)C(=O)OC)c2cc(F)ccc21] | 1.81 | 413.2 | C |
| 51 | 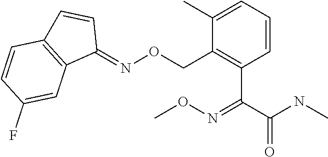 [SMILES: CNC(=O)/C(=N/OC)c1cccc(C)c1CO/N=C1\C=Cc2ccc(F)cc21] | 1.77 | 382.1 | C |
| 52 | 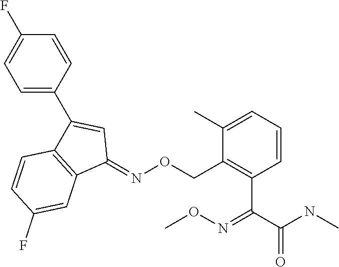 [SMILES: CNC(=O)/C(=N/OC)c1cccc(C)c1CO/N=C1\C=C(c2ccc(F)cc2)c2ccc(F)cc21] | 1.81 | 476.1 | C |
| 53 | 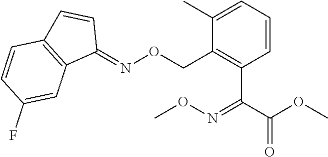 [SMILES: CO/N=C(/C(=O)OC)c1cccc(C)c1CO/N=C1\C=Cc2ccc(F)cc21] | 1.86 | 383.1 | C |
| 54 | 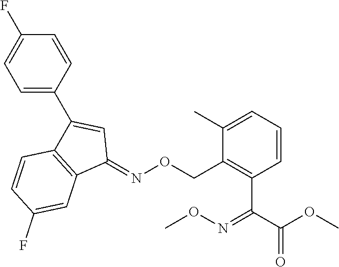 [SMILES: CO/N=C(/C(=O)OC)c1cccc(C)c1CO/N=C1\C=C(c2ccc(F)cc2)c2ccc(F)cc21] | 2.07 | 477.2 | C |
| 55 | 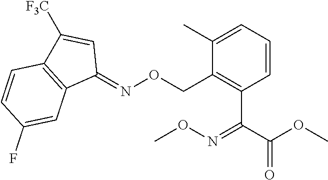 [SMILES: CO/N=C(/C(=O)OC)c1cccc(C)c1CO/N=C1\C=C(C(F)(F)F)c2ccc(F)cc21] | 1.90 | 451.1 | C |
| 56 | 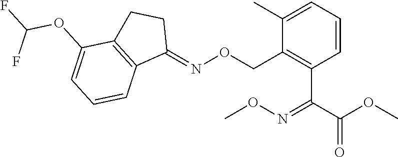 [SMILES: CO/N=C(/C(=O)OC)c1cccc(C)c1CO/N=C1\CCc2c(OC(F)F)cccc21] | 1.85 | 433.1 | C |
| 57 | 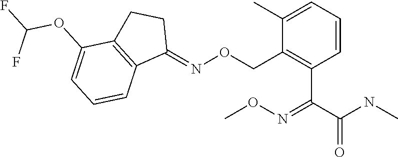 [SMILES: CNC(=O)/C(=N/OC)c1cccc(C)c1CO/N=C1\CCc2c(OC(F)F)cccc21] | 1.75 | 432.2 | C |
| 58 | 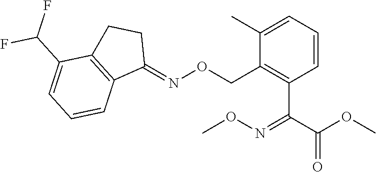 [SMILES: CO/N=C(/C(=O)OC)c1cccc(C)c1CO/N=C1\CCc2c1cccc2C(F)F] | 1.83 | 417.2 | C |
| 59 | 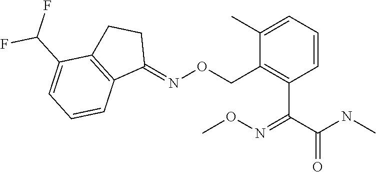 [SMILES: CNC(=O)/C(=N/OC)c1cccc(C)c1CO/N=C1\CCc2c1cccc2C(F)F] | 1.74 | 416.2 | C |
| 60 | 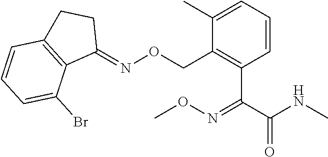 [SMILES: CNC(=O)/C(=N/OC)c1cccc(C)c1CO/N=C1\CCc2cccc(Br)c21] | 1.27 | 446 | B |
| 61 | 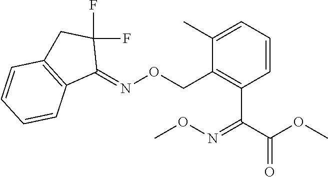 [SMILES: CO/N=C(/C(=O)OC)c1cccc(C)c1CO/N=C1/c2ccccc2CC1(F)F] | | | |
| 62 | 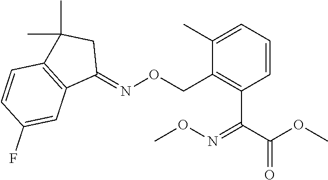 [SMILES: CO/N=C(/C(=O)OC)c1cccc(C)c1CO/N=C1\CC(C)(C)c2ccc(F)cc21] | 2.21 | 381 | A |
| 63 | 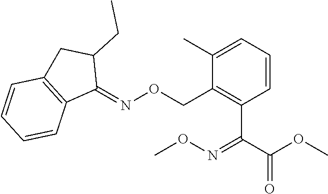 [SMILES: CCC1Cc2ccccc2/C1=N/OCc1c(C)cccc1/C(=N\OC)C(=O)OC] | 1.39 | 395 | B |
| 64 | 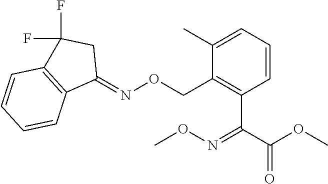 [SMILES: CO/N=C(/C(=O)OC)c1cccc(C)c1CO/N=C1\CC(F)(F)c2ccccc21] | 1.74 | 403.1 | C |
| 65 | 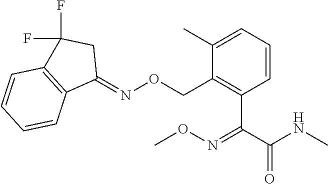 [SMILES: CNC(=O)/C(=N/OC)c1cccc(C)c1CO/N=C1\CC(F)(F)c2ccccc21] | 1.64 | 402.1 | C |
| 66 | 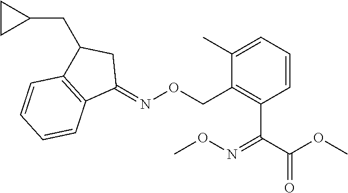 [SMILES: CO/N=C(/C(=O)OC)c1cccc(C)c1CO/N=C1\CC(CC2CC2)c2ccccc21] | 1.91 | 439.2 | C |
| 67 | 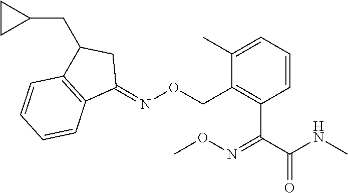 [SMILES: CNC(=O)/C(=N/OC)c1cccc(C)c1CO/N=C1\CC(CC2CC2)c2ccccc21] | 1.82 | 438.2 | C |

### Biological Studies

### Green House

The compound was dissolved in a mixture of acetone and/or dimethylsulfoxide and the wetting agent/emulsifier Wettol, which is based on ethoxylated alkylphenoles, in a ratio (volume) solvent-emulsifier of 99 to 1 to give a total volume of 5 ml. Subsequently, water was added to total volume of 100 ml. This stock solution was then diluted with the described solvent-emulsifier-water mixture to the final concentration given in the table below.

### Use Example 1. Protective Control of Soybean Rust on Soybeans Caused by Phakopsora pachyrhizi (PHAKPA P2)

Leaves of potted soybean seedlings were sprayed to run-off with the previously described spray solution, containing the concentration of active ingredient or their mixture as described below. The plants were allowed to air-dry. The trial plants were cultivated for 2 days in a greenhouse chamber at 23-27° C. and a relative humidity between 60 and 80%. Then the plants were inoculated with spores of Phakopsora pachyrhizi. The strain used contains the amino acid substitution F129L in the mitochondrial cytochrome b protein conferring resistance to Qo inhibitors. To ensure the success the artificial inoculation, the plants were transferred to a humid chamber with a relative humidity of about 95% and 20 to 24° C. for 24 hr. The trial plants were cultivated for up to 14 days in a greenhouse chamber at 23 to 27° C. and a relative humidity between 60 and 80%. The extent of fungal attack on the leaves was visually assessed as % diseased leaf area, the disease level of untreated controls was usually higher than 85%.

### Use Example 2. Protective Control of Soybean Rust on Soybeans Caused by Phakopsora pachyrhizi (PHAKPA P6)

Leaves of potted soybean seedlings were sprayed to run-off with the previously described spray solution, containing the concentration of active ingredient as described below. The plants were allowed to air-dry. The trial plants were cultivated for six days in a greenhouse chamber at 23-27° C. and a relative humidity between 60 and 80%. Then the plants were inoculated with spores of Phakopsora pachyrhizi. The strain used contains the amino acid substitution F129L in the mitochondrial cytochrome b protein conferring resistance to Qo inhibitors. To ensure the success the artificial inoculation, the plants were transferred to a humid chamber with a relative humidity of about 95% and 23 to 27° C. for 24 hr. The trial plants were cultivated for up to 14 days in a greenhouse chamber at 23 to 27° C. and a relative humidity between 60 and 80%. The extent of fungal attack on the leaves was visually assessed as % diseased leaf area, the disease level of untreated controls was usually higher than 85%.

The results of the abovementioned use examples are given in the following Table 1. All test results in Table 1 are given for the control of phytopathogenic fungi containing the amino acid substitution F129L in the mitochondrial cytochrome b protein conferring resistance to Qo inhibitors.

TABLE 1

| | | PHAKPA (F129L) Disease level (%) | | | |
|---|---|---|---|---|---|
| Compound | | P2 at | P6 at | P2 at | P6 at |
| No. | Structure | 16 ppm | 16 ppm | 4 ppm | 4 ppm |
| 1 | 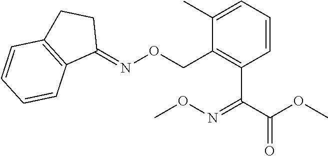 [SMILES: CO/N=C(/C(=O)OC)c1cccc(C)c1CO/N=C1\CCc2ccccc21] | 8 | 1 | 28 | 25 |
| 2 | 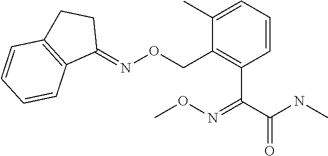 [SMILES: CNC(=O)/C(=N/OC)c1cccc(C)c1CO/N=C1\CCc2ccccc21] | 1 | 12 | 6 | 42 |
| 3 | 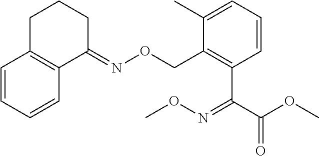 [SMILES: CO/N=C(/C(=O)OC)c1cccc(C)c1CO/N=C1\CCCc2ccccc21] | 100 | 90 | 100 | 100 |
| 4 | 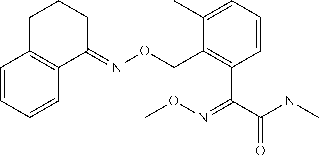 [SMILES: CNC(=O)/C(=N/OC)c1cccc(C)c1CO/N=C1\CCCc2ccccc21] | 7 | 32 | 90 | 87 |
| 5 | 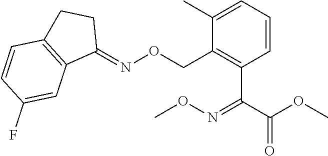 [SMILES: CO/N=C(/C(=O)OC)c1cccc(C)c1CO/N=C1\CCc2ccc(F)cc21] | 3 | 1 | 30 | 40 |
| 6 | 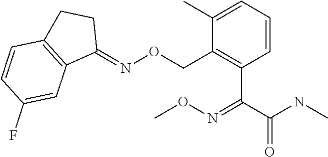 [SMILES: CNC(=O)/C(=N/OC)c1cccc(C)c1CO/N=C1\CCc2ccc(F)cc21] | 0 | 0 | 3 | 20 |
| 7 | 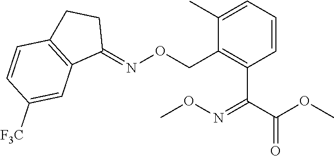 [SMILES: CO/N=C(/C(=O)OC)c1cccc(C)c1CO/N=C1\CCc2ccc(C(F)(F)F)cc21] | 10 | 5 | 25 | 30 |
| 8 | 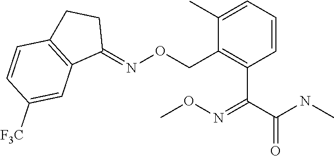 [SMILES: CNC(=O)/C(=N/OC)c1cccc(C)c1CO/N=C1\CCc2ccc(C(F)(F)F)cc21] | 0 | 0 | 1 | 5 |
| 9 | 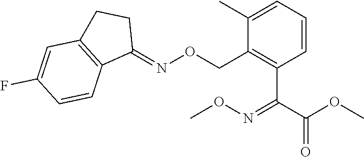 [SMILES: CO/N=C(/C(=O)OC)c1cccc(C)c1CO/N=C1\CCc2cc(F)ccc21] | 7 | 20 | 40 | 90 |
| 10 | 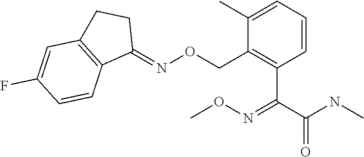 [SMILES: CNC(=O)/C(=N/OC)c1cccc(C)c1CO/N=C1\CCc2cc(F)ccc21] | 1 | 25 | 25 | 90 |
| 12 | 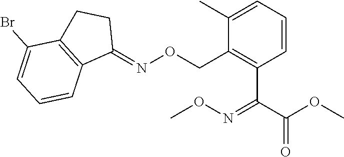 [SMILES: CO/N=C(/C(=O)OC)c1cccc(C)c1CO/N=C1\CCc2c(Br)cccc21] | 97 | 100 | 100 | 100 |
| 13 | 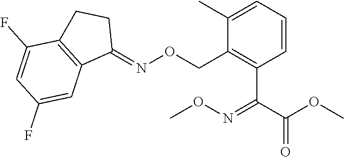 [SMILES: CO/N=C(/C(=O)OC)c1cccc(C)c1CO/N=C1\CCc2c(F)cc(F)cc21] | 8 | 20 | 53 | 83 |
| 15 | 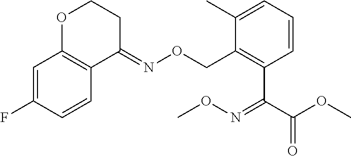 [SMILES: CO/N=C(/C(=O)OC)c1cccc(C)c1CO/N=C1\CCOc2cc(F)ccc21] | 25 | 25 | 80 | 90 |
| 16 | 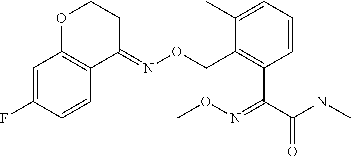 [SMILES: CNC(=O)/C(=N/OC)c1cccc(C)c1CO/N=C1\CCOc2cc(F)ccc21] | 2 | 0 | 17 | 22 |
| 17 | 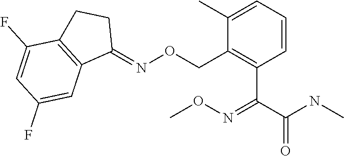 [SMILES: CNC(=O)/C(=N/OC)c1cccc(C)c1CO/N=C1\CCc2c(F)cc(F)cc21] | 8 | 4 | 26 | 25 |
| 18 | 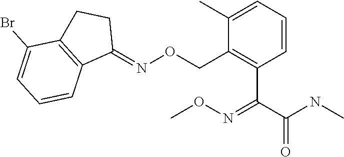 [SMILES: CNC(=O)/C(=N/OC)c1cccc(C)c1CO/N=C1\CCc2c(Br)cccc21] | 27 | 73 | 67 | 90 |
| 19 | 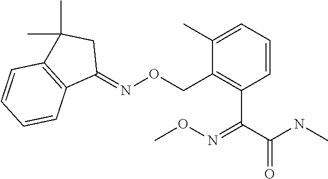 [SMILES: CNC(=O)/C(=N/OC)c1cccc(C)c1CO/N=C1\CC(C)(C)c2ccccc21] | 90 | 100 | 100 | 100 |
| 20 | 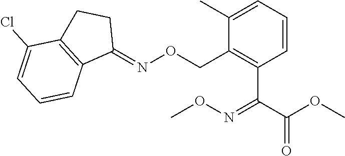 [SMILES: CO/N=C(/C(=O)OC)c1cccc(C)c1CO/N=C1\CCc2c(Cl)cccc21] | 70 | 93 | 97 | 100 |
| 21 | 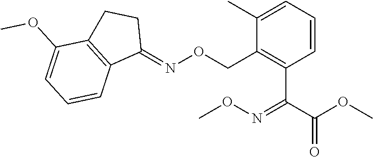 | 4 | 11 | 31 | 36 |
| 22 | 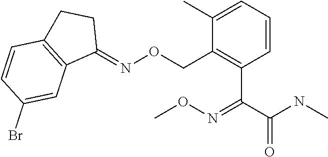 [SMILES: CNC(=O)/C(=N/OC)c1cccc(C)c1CO/N=C1\CCc2ccc(Br)cc21] | 6 | 10 | 22 | 37 |
| 23 | 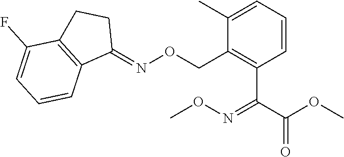 [SMILES: CO/N=C(/C(=O)OC)c1cccc(C)c1CO/N=C1\CCc2c(F)cccc21] | 18 | 32 | 53 | 70 |
| 25 | 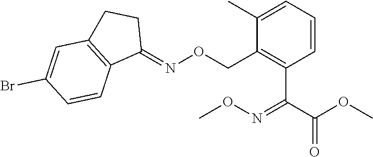 [SMILES: CO/N=C(/C(=O)OC)c1cccc(C)c1CO/N=C1\CCc2cc(Br)ccc21] | 32 | 60 | 57 | 93 |
| 26 | 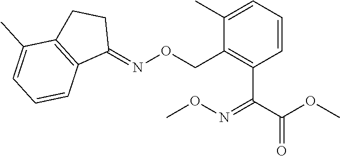 | 57 | 83 | 80 | 90 |
| 27 | 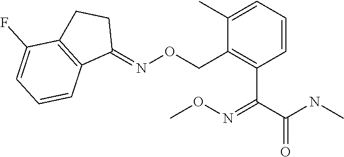 [SMILES: CNC(=O)/C(=N/OC)c1cccc(C)c1CO/N=C1\CCc2c(F)cccc21] | 1 | 10 | 13 | 67 |
| 28 | 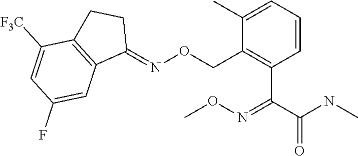 [SMILES: CNC(=O)/C(=N/OC)c1cccc(C)c1CO/N=C1\CCc2c1cc(F)cc2C(F)(F)F] | 100 | 100 | 70 | 100 |
| 29 | 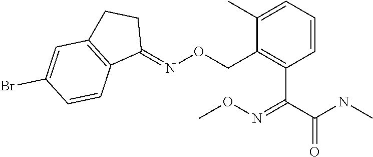 [SMILES: CNC(=O)/C(=N/OC)c1cccc(C)c1CO/N=C1\CCc2cc(Br)ccc21] | 8 | 22 | 37 | 70 |
| 30 | 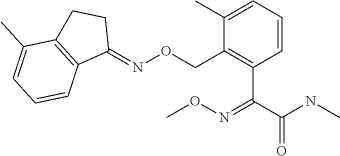 | 3 | 32 | 47 | 93 |
| 31 | 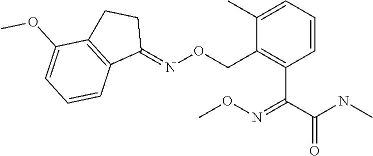 | 0 | 1 | 3 | 35 |
| 32 | 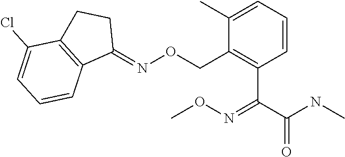 [SMILES: CNC(=O)/C(=N/OC)c1cccc(C)c1CO/N=C1\CCc2c(Cl)cccc21] | 3 | 4 | 23 | 53 |
| 33 | 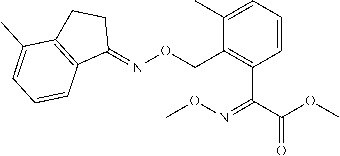 | 57 | 60 | 100 | 97 |
| 34 | 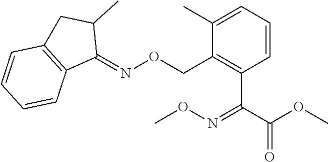 [SMILES: CO/N=C(/C(=O)OC)c1cccc(C)c1CO/N=C1/c2ccccc2CC1C] | 93 | 97 | 100 | 100 |
| 38 | 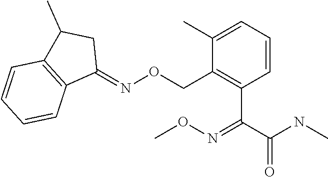 [SMILES: CNC(=O)/C(=N/OC)c1cccc(C)c1CO/N=C1\CC(C)c2ccccc21] | 3 | 18 | 28 | 80 |
| 40 | 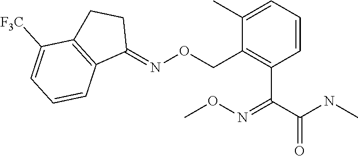 [SMILES: CNC(=O)/C(=N/OC)c1cccc(C)c1CO/N=C1\CCc2c1cccc2C(F)(F)F] | 83 | 93 | 100 | 100 |
| 43 | 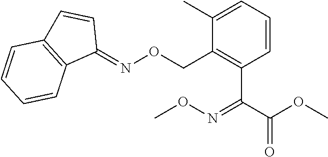 [SMILES: CO/N=C(/C(=O)OC)c1cccc(C)c1CO/N=C1\C=Cc2ccccc21] | 100 | 100 | 100 | 100 |
| 44 | 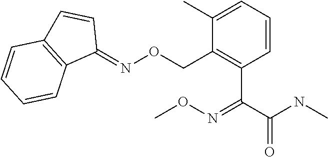 [SMILES: CNC(=O)/C(=N/OC)c1cccc(C)c1CO/N=C1\C=Cc2ccccc21] | 100 | 100 | 100 | 100 |
| 45 | 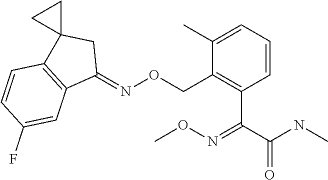 [SMILES: CNC(=O)/C(=N/OC)c1cccc(C)c1CO/N=C1\CC2(CC2)c2ccc(F)cc21] | 93 | 100 | 100 | 90 |
| 46 | 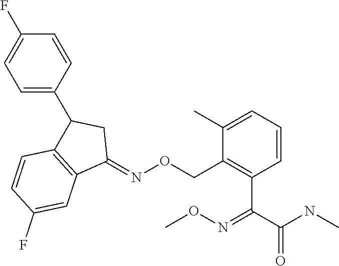 [SMILES: CNC(=O)/C(=N/OC)c1cccc(C)c1CO/N=C1\CC(c2ccc(F)cc2)c2ccc(F)cc21] | 100 | 93 | 100 | 97 |
| 47 | 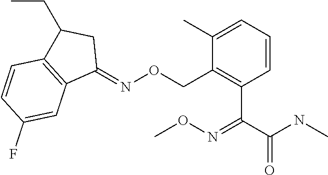 [SMILES: CCC1C/C(=N\OCc2c(C)cccc2/C(=N\OC)C(=O)NC)c2cc(F)ccc21] | 27 | 57 | 93 | 93 |
| 48 | 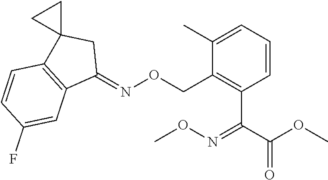 [SMILES: CO/N=C(/C(=O)OC)c1cccc(C)c1CO/N=C1\CC2(CC2)c2ccc(F)cc21] | 100 | 100 | 100 | 100 |
| 49 | 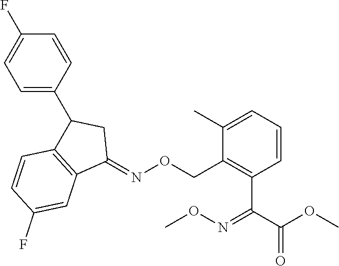 [SMILES: CO/N=C(/C(=O)OC)c1cccc(C)c1CO/N=C1\CC(c2ccc(F)cc2)c2ccc(F)cc21] | 100 | 100 | 100 | 90 |
| 50 | 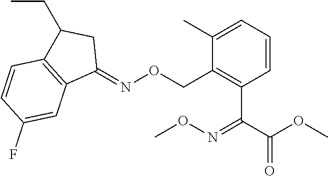 [SMILES: CCC1C/C(=N\OCc2c(C)cccc2/C(=N\OC)C(=O)OC)c2cc(F)ccc21] | 87 | 73 | 100 | 93 |
| 51 | 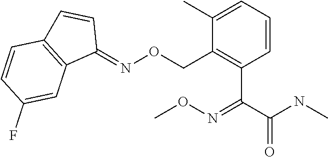 [SMILES: CNC(=O)/C(=N/OC)c1cccc(C)c1CO/N=C1\C=Cc2ccc(F)cc21] | 13 | 47 | 60 | 90 |
| 53 | 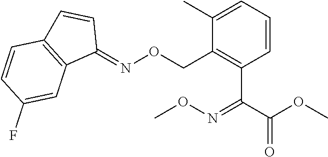 [SMILES: CO/N=C(/C(=O)OC)c1cccc(C)c1CO/N=C1\C=Cc2ccc(F)cc21] | 57 | 53 | 97 | 80 |
| 54 | 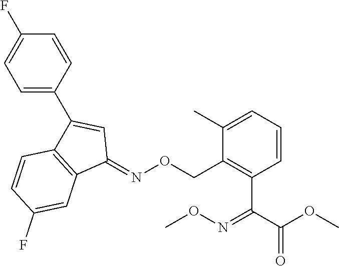 [SMILES: CO/N=C(/C(=O)OC)c1cccc(C)c1CO/N=C1\C=C(c2ccc(F)cc2)c2ccc(F)cc21] | 100 | 87 | 100 | 87 |
| 55 | 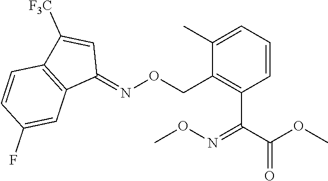 [SMILES: CO/N=C(/C(=O)OC)c1cccc(C)c1CO/N=C1\C=C(C(F)(F)F)c2ccc(F)cc21] | 100 | 93 | 100 | 87 |
| 56 | 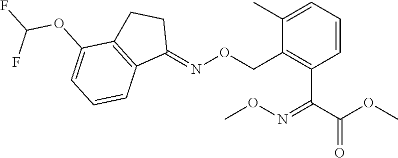 [SMILES: CO/N=C(/C(=O)OC)c1cccc(C)c1CO/N=C1\CCc2c(OC(F)F)cccc21] | 93 | 87 | 100 | 97 |
| 57 | 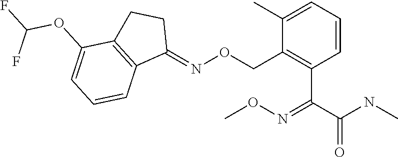 [SMILES: CNC(=O)/C(=N/OC)c1cccc(C)c1CO/N=C1\CCc2c(OC(F)F)cccc21] | 22 | 33 | 70 | 77 |
| 58 | 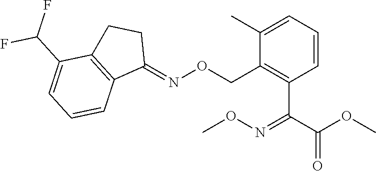 [SMILES: CO/N=C(/C(=O)OC)c1cccc(C)c1CO/N=C1\CCc2c1cccc2C(F)F] | 100 | 90 | 100 | 93 |
| 59 | 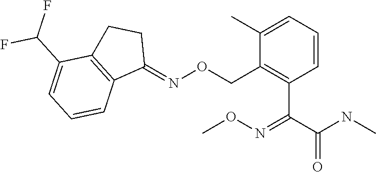 [SMILES: CNC(=O)/C(=N/OC)c1cccc(C)c1CO/N=C1\CCc2c1cccc2C(F)F] | 97 | 67 | 100 | 87 |
| 60 | 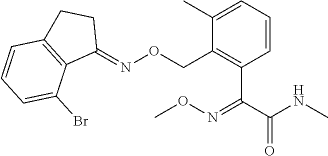 [SMILES: CNC(=O)/C(=N/OC)c1cccc(C)c1CO/N=C1\CCc2cccc(Br)c21] | 93 | 63 | 100 | 87 |
| 61 | 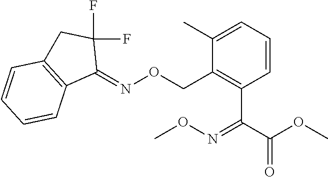 [SMILES: CO/N=C(/C(=O)OC)c1cccc(C)c1CO/N=C1/c2ccccc2CC1(F)F] | 87 | 80 | 100 | 90 |
| 63 | 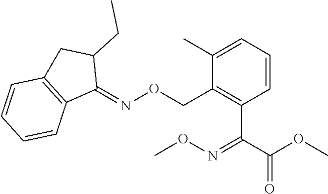 [SMILES: CCC1Cc2ccccc2/C1=N/OCc1c(C)cccc1/C(=N\OC)C(=O)OC] | 100 | 87 | 100 | 90 |

### Comparative Trials

TABLE C1

| | | PHAKPA (F129L) Disease level (%) | | | |
|---|---|---|---|---|---|
| | | P2 at | P2 at | P6 at | P6 at |
| Compound | Structure | 4 ppm | 16 ppm | 4 ppm | 16 ppm |
| Trifloxystrobin as comparative example | 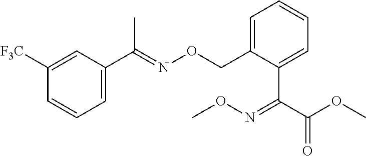 [SMILES: CO/N=C(/C(=O)OC)c1ccccc1CO/N=C(\C)c1cccc(C(F)(F)F)c1] | 83 | 24 | 100 | 67 |
| Ex. 7 | 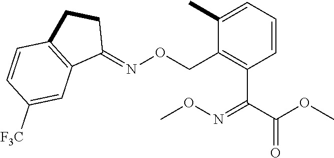 | 27 | 9 | 25 | 4 |

The results in Table C1 show that the specific substituent at position R³ the improves the fungicidal activity against phytopathogenic fungi containing the amino acid substitution F129L in the mitochondrial cytochrome b protein conferring resistance to Qo inhibitors compared to trifloxystrobin where the position R³ is unsubstituted.

1. A compound of formula I 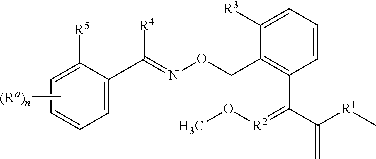
wherein
R¹ is selected from 0 and NH;
R² is selected from CH and N;
R³ is selected from halogen, CN, C₁-C₄-alkyl, C₂-C₄-alkenyl, C₂-C₄-alkynyl, C₁-C₄-haloalkyl, C₂-C₄-haloalkenyl, C₂-C₄-haloalkynyl, C₃-C₆-cycloalkyl, —O—C₁-C₄-alkyl, —O—C₁-C₄-haloalkyl, —O—C₃-C₆-cycloalkyl, —C₁-C₂-alkyl-C₃-C₆-cycloalkyl, phenyl, 3- to 6-membered heterocycloalkyl and 5- or 6-membered heteroaryl,
wherein said heterocycloalkyl and heteroaryl besides carbon atoms contain 1, 2 or 3 heteroatoms selected from N, O and S provided that such heterocycloalkyl and heteroaryl cannot contain 2 contiguous atoms selected from O and S,
wherein said phenyl, heterocycloalkyl and heteroaryl are bound directly or via an oxygen atom or via a C₁-C₂-alkylene linker, and wherein said phenyl and heteroaryl are unsubstituted or substituted by 1, 2 or 3 identical or different substituents selected from halogen, CN, NH₂, NO₂, C₁-C₄-alkyl, C₁-C₄-haloalkyl, —O—C₁-C₄-alkyl and —O—C₁-C₄-haloalkyl;
R⁴ and R⁵, together with the three interjacent carbon atoms, form a partially unsaturated 5- to 6-membered carbo- or heterocycle,
wherein the heterocycle includes beside carbon atoms 1, 2 or 3 heteroatoms independently selected from N, O and S as ring member atoms provided that such heterocycle cannot contain 2 contiguous atoms selected from O and S; and wherein the carbo- or heterocycle is unsubstituted or carries 1, 2 or up to the maximum possible number of identical or different groups R⁴⁵; wherein
R⁴⁵ is selected from halogen, CN, C₁-C₄-alkyl, C₁-C₄-haloalkyl, phenyl, C₃-C₆-cycloalkyl and —C₁-C₂-alkyl-C₃-C₆-cycloalkyl;
wherein it is possible that two R⁴⁵ substituents which are bound to the same carbon atom or to two adjacent carbon atoms form a saturated 3- to 5-membered carbocycle;
and wherein the cyclic moieties of R⁴⁵ are unsubstituted or carry 1, 2 or 3 identical or different groups R^{45b}:
R^{45b} is selected from halogen, CN, NH₂, NO₂, C₁-C₄-alkyl, C₁-C₄-haloalkyl, —O—C₁-C₄-alkyl and —O—C₁-C₄-haloalkyl;
R^{a} is selected from halogen, CN, —NR⁵R⁶, C₁-C₄-alkyl, C₂-C₄-alkenyl, C₂-C₄-alkynyl, —O—C₁-C₄-alkyl, —C(═N—O—C₁-C₄-alkyl)-C₁-C₄-alkyl, —C(═O)—C₁-C₄-alkyl, —O—CH₂—C(═N—O—C₁-C₄-alkyl)-C₁-C₄-alkyl, C₃-C₆-cycloalkyl, C₃-C₆-cycloalkenyl, —C₁-C₂-alkyl-C₃-C₆-cycloalkyl, —O—C₃-C₆-cycloalkyl, phenyl, 3- to 6-membered heterocycloalkyl, 3- to 6-membered heterocycloalkenyl and 5- or 6-membered heteroaryl,
wherein said heterocycloalkyl, heterocycloalkenyl and heteroaryl besides carbon atoms contain 1, 2 or 3 heteroatoms selected from N, O and S provided that such heterocycloalkyl, heterocycloalkenyl and heteroaryl cannot contain 2 contiguous atoms selected from O and S,
wherein said phenyl, heterocycloalkyl, heterocycloalkenyl and heteroaryl are bound directly or via an oxygen atom or via a C₁-C₂-alkylene linker, and wherein the aliphatic and cyclic moieties of R^{a} are unsubstituted or carry 1, 2, 3, 4 or up to the maximum number of identical or different groups R^{b}:
R^{b} is selected from halogen, CN, NH₂, NO₂, C₁-C₄-alkyl, C₁-C₄-haloalkyl, —O—C₁-C₄-alkyl and —O—C₁-C₄-haloalkyl;
R⁵, R⁶ are independently of each other selected from the group consisting of H, C₁-C₆-alkyl, C₁-C₆-haloalkyl and C₂-C₄-alkynyl;
n is an integer selected from 0, 1, 2, 3 and 4;
and in form of stereoisomers and tautomers thereof, and the N-oxides and the agriculturally acceptable salts thereof.

2. The compound according to claim 1, wherein R¹ is selected from 0 and NH; and R² is selected from CH and N, provided that R² is N in case R¹ is NH.

3. The compound according to claim 1, wherein R³ is selected from halogen, C₁-C₂-alkyl, C₁-C₂-haloalkyl, C₃-C₄-cycloalkyl, —O—C₁-C₂-alkyl and —O—C₁-C₂-haloalkyl.

4. The compound according to claim 3, wherein R³ is selected from halogen, C₁-C₂-alkyl and C₁-C₂-haloalkyl.

5. The compound according to claim 1, wherein R^{a} is selected from halogen, C₁-C₃-alkyl, C₂-C₃-alkenyl, C₂-C₃-alkynyl, —O—C₁-C₃-alkyl, —C(═N—O—C₁-C₂-alkyl)-C₁-C₂-alkyl, —O—CH₂—C(═N—O—C₁-C₂-alkyl)-C₁-C₂-alkyl, C₃-C₄-cycloalkyl, —C₁-C₂-alkyl-C₃-C₄-cycloalkyl, —O—C₃-C₄-cycloalkyl, phenyl, 3- to 5-membered heterocycloalkyl and 5- or 6-membered heteroaryl, wherein said heterocycloalkyl and heteroaryl besides carbon atoms contain 1 or 2 heteroatoms selected from N, O and S provided that such heterocycloalkyl and heteroaryl cannot contain 2 contiguous atoms selected from O and S, wherein said phenyl, heterocycloalkyl and heteroaryl are bound directly or via an oxygen atom or via a methylene linker, and wherein the aliphatic and cyclic moieties of R^{a} are unsubstituted or carry 1, 2 or 3 of identical or different groups R^{b} which independently of one another are selected from halogen, CN, methyl and C₁-haloalkyl.

6. The compound according to claim 5, wherein R^{a} is selected from halogen, C₁-C₃-alkyl, —O—C₁-C₃-alkyl, C₃-C₄-cycloalkyl and phenyl, and wherein the aliphatic and cyclic moieties of R^{a} are unsubstituted or carry 1, 2 or 3 of identical or different groups R^{b} which independently of one another are selected from halogen, methyl and C₁-haloalkyl.

7. The compound according to claim 1, wherein n is 0, 1 or 2.

8. The compound according to claim 1, wherein R⁴ and R⁵, together with the three interjacent carbon atoms, form a partially unsaturated 5- to 6-membered carbocycle, wherein said carbocycle is unsubstituted or carries 1 or 2 identical or different groups R⁴⁵, wherein R⁴⁵ is selected from halogen, C₁-C₄-alkyl, C₁-C₄-haloalkyl, phenyl and C₃-C₆-cycloalkyl, wherein it is possible that two R⁴⁵ substituents which are bound to the same carbon atom form a cyclopropyl ring.

9. The compound according to claim 8, wherein R⁴ and R⁵, together with the three interjacent carbon atoms, form a cyclopentene ring, wherein said cyclopentene ring is unsubstituted or carries 1 or 2 identical or different groups R⁴⁵, wherein R⁴⁵ is selected from halogen, C₁-C₄-alkyl, C₁-C₄-haloalkyl, phenyl and C₃-C₆-cycloalkyl, wherein it is possible that two R⁴⁵ substituents which are bound to the same carbon atom form a cyclopropyl ring.

10. An agrochemical composition comprising an auxiliary and at least one compound of formula I, as defined in claim 1 or in the form of a stereoisomer or an agriculturally acceptable salt or a tautomer or N-oxide thereof.

11. (canceled)

12. (canceled)

13. A method for combating phytopathogenic fungi comprising:
treating curatively and/or preventively a plant or plant propagation material of said plant that is at risk of being diseased from the said phytopathogenic fungi, and/or applying to the said phytopathogenic fungi, at least one compound of formula I as defined in claim 1.

14. The method for combating phytopathogenic fungi according to claim 13 wherein said phytopathogenic fungi contain an amino acid substitution F129L in the mitochondrial cytochrome b protein conferring resistance to Qo inhibitors.

15. The method according to claim 13, wherein the phytopathogenic fungi are soybean rust (Phakopsora pachyrhizi and/or P. meibomiae).
